(19) United States
(12) Patent Application Publication
(10) Pub. No.: US 2024/0400986 A1
(43) Pub. Date: Dec. 5, 2024

(54) MEMBRANE BOUND COMPOSITIONS AND METHODS RELATING TO SAME

(71) Applicant: FLAGSHIP PIONEERING INNOVATIONS VII, LLC, Cambridge, MA (US)

(72) Inventors: Michael Ka Chun Wong, Arlington, MA (US); Anna Pensalfini, Cambridge, MA (US); David Barry Kolesky, Arlington, MA (US); Kyle Ping Chiang, Arlington, MA (US); Rakshita Anilkanth Charan, Arlington, MA (US)

(21) Appl. No.: 18/690,845

(22) PCT Filed: Sep. 15, 2022
(86) PCT No.: PCT/US2022/043686

§ 371 Date: Mar. 11, 2024

Related U.S. Application Data
(60) Provisional application No. 63/348,192, filed on Jun. 2, 2022.
(60) Provisional application No. 63/244,580, filed on Sep. 15, 2021.

(51) Int. Cl. C12N 5/0793; C12N 5/077

(52) U.S. Cl. CPC C12N 5/0619; C12N 5/0657; C12N 2501/999; C12N 2502/086

(57) ABSTRACT

This disclosure relates generally to membrane-bound compositions, in particular exophers having a diameter between 1 and 20 microns induced from human cells, and uses thereof.

### RELATED APPLICATIONS

This application claims priority to U.S. Ser. No. 63/244,580, filed Sep. 15, 2021 and U.S. Ser. No. 63/348,192, filed Jun. 2, 2022, the entire contents of each of which is incorporated herein by reference.

### BACKGROUND

There is an ongoing need to develop novel interventions for modulating cellular health.

### SUMMARY

Exophers have been described as large, membrane-bound extracellular bodies released from cells in C. elegans and mice, potentially involved in removal of nonessential products, e.g., waste products, from cells. However, exophers from human cells have not been described in the literature. The present disclosure provides, e.g., methods of making preparations of membrane bound bodies from mammalian cells, e.g., human, cells, by inducing a process of, or similar to, exophoresis, and using such preparations, e.g., to deliver cargo to target cells. In some embodiments, the large, membrane bound bodies are gigasomes. The preparations described herein may comprise exogenous cargo such as recombinant proteins or nucleic acids.

Additional features of any of the aforesaid compositions or methods include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

### ENUMERATED EMBODIMENTS

1. A method of making or manufacturing a gigasome preparation, comprising:
providing a volume comprising:
   (i) a population of producer cells, wherein the producer cells are human cells; and
   (ii) a medium;
maintaining (e.g., culturing) the population of producer cells under conditions that allow for exopheresis, wherein the producer cells are viable after the exopheresis, and
enriching membrane-bound bodies on the basis of having a diameter between about 1-20 μm from the volume (e.g., from the medium),
thereby making or manufacturing a gigasome preparation.

2. A method of inducing release, from a population of producer cells, of membrane-bound bodies comprising nonessential products from the population of producer cells, comprising:
providing a volume comprising:
   (i) a population of producer cells, wherein the producer cells are human cells; and
   (ii) a medium;
maintaining (e.g., culturing) the population of producer cells under conditions that allow for release of membrane-bound bodies from the producer cells, wherein the membrane-bound bodies comprise one or more products nonessential to the producer cells; and
enriching membrane-bound bodies on the basis of comprising the one or more nonessential products (e.g., from the medium),
thereby inducing release of membrane-bound bodies comprising nonessential products from the population of producer cells;
optionally wherein the membrane-bound bodies comprise organelles (e.g., mitochondria, e.g., dysfunctional mitochondria, or lysosomes), protein aggregates, lipids, protein translation machinery, ribosomes, cytoplasm or nonessential components or constituents thereof, nonessential metabolites, nonessential small molecules, nonessential nucleic acid molecules (e.g., mRNAs, miRNAs, or siRNAs), or nonessential carbohydrates (e.g., sugars or glycans); and
optionally wherein the membrane-bound bodies have diameters between about 1-20 μm.

3. The method of embodiment 1 or 2, wherein the method is performed in vitro.

4. The method of embodiment 1 or 2, wherein the method is performed ex vivo.

5. The method of any of embodiments 1-4, wherein the maintaining is under conditions whereby the producer cells do not substantially undergo cell death (e.g., apoptosis or necrosis).

6. The method of any of embodiments 1-5, wherein the producer cell is stressed compared to a reference cell (e.g., an otherwise similar cell that is not maintained under conditions that allow for exopheresis and/or conditions that allow for release of membrane-bound bodies from the producer cells, wherein the membrane-bound bodies comprise one or more nonessential products).

7. The method of embodiment 6, wherein the producer cell stress is proteotoxic stress.

8. The method of embodiment 6, wherein the producer cell has impaired autophagy.

9. The method of embodiment 6, wherein the producer cell has higher levels of autophagy relative to an otherwise similar cell that is not maintained under conditions that allow for exopheresis and/or conditions that allow for release of membrane-bound bodies from the producer cells, wherein the membrane-bound bodies comprise one or more nonessential products.

10. The method of embodiment 9, wherein the higher levels of autophagy result in the membrane-bound bodies comprising higher levels of LC3-II relative to the producer cell.

11. The method of of embodiment 6, wherein the producer cell has a downregulated mTOR pathway relative to an otherwise similar cell that is not maintained under conditions that allow for exopheresis and/or conditions that allow for release of membrane-bound bodies from the producer cells, wherein the membrane-bound bodies comprise one or more nonessential products.

12. The method of embodiment 6, wherein the producer cell has a higher metabolic activity than an otherwise similar cell that is not maintained under conditions that allow for exopheresis and/or conditions that allow for release of membrane-bound bodies from the producer cells, wherein the membrane-bound bodies comprise one or more nonessential products.

13. The method of any of the preceding embodiments, wherein the maintaining is under conditions whereby no more than 10%, 20%, 30%, 40%, or 50% of the producer cells undergo cell death (e.g., apoptosis or necrosis), e.g., over a period of 6, 12, 24, 36, 48, 60, or 72 hours.

14. The method of any of the preceding embodiments, wherein at least 50%, 60%, 70%, 80%, 90%, 95%, or 100% of the producer cells remain viable after exopheresis.

15. The method of any of the preceding embodiments, wherein at least 50%, 60%, 70%, 80%, 90%, 95%, or 100% of the producer cells do not comprise detectable levels of an apoptotic marker after exopheresis.

16. The method of any of the preceding embodiments, wherein at least 50%, 60%, 70%, 80%, 90%, 95%, or 100% of the producer cells are negative for apoptosis according to an apoptosis assay, e.g., a TUNEL assay or an annexin V assay.

17. The method of any of the preceding embodiments, wherein at least 50%, 60%, 70%, 80%, 90%, 95%, or 100% of the producer cells do not comprise increased levels of an apoptotic marker after exopheresis relative to an otherwise identical producer cell prior to exopheresis.

18. The method of any of embodiments 15-17, wherein the apoptotic marker comprises increased caspase (e.g., caspase-3) activity, DNA degradation (e.g., as determined by a TUNEL assay), or surface-exposed phosphatidylserine (e.g., as determined by an annexin V assay).

19. The method of any of the preceding embodiments, wherein the maintaining comprises incubating the producer cells under conditions suitable for inducing exopheresis (e.g., inducing the production of about 1, 2, 3, 4, or 5 gigasomes or membrane bound bodies per producer cell).

20. The method of any of the preceding embodiments, wherein the maintaining comprises incubating the producer cells under conditions suitable for continuous exopheresis (e.g., wherein each producer cell produces at least about 2, 3, 4, 5, 6, 7, 8, 9, or 10 gigasomes or membrane bound bodies, e.g., over the course of at least 1, 2, 3, 4, 5, 6, 7, 8, 9, or 10 days).

21. The method of any of the preceding embodiments, wherein the producer cells are maintained (e.g., cultured) in a monoculture.

22. The method of any of the preceding embodiments, wherein the producer cell is selected from a neuron (e.g., a HCN2 cell, or a HT22 cell), a neuroblastoma cell (e.g., an SH-SY5Y cell), a neural progenitor cell, a muscle cell, (e.g., a cardiac muscle cell), a stem cell (e.g., an induced pluripotent stem cell (iPSC)), an endothelial cell (e.g., a microvascular endothelial cell, e.g., a cerebral microvascular endothelial cell), HBEC-5i, ReNcell CX, or iCell GlutaNeurons.

23. The method of any of the preceding embodiments, wherein the producer cells are primary cells (e.g., neuronal cells, neural progenitor cells, muscle cells (e.g., cardiac muscle cells), endothelial cells, or stem cells).

24. The method of any of the preceding embodiments, wherein the producer cells are maintained (e.g., cultured) with a second cell type (e.g., in co-culture).

25. The method of embodiment 24, wherein the second cell type is selected from a macrophage (e.g., THP-1) and a microglial cell (e.g., iCell Microglia, Huμglia, CHME-5, HMO6, and HMC3).

26. The method of embodiment 24, wherein the producer cells and the second cell type (e.g., macrophages) are physically separated (e.g., using a transwell or removable separater).

27. The method of any of the preceding embodiments, wherein the producer cells are maintained in an organoid system.

28. The method of any of the preceding embodiments, wherein each producer cell produces, on average, at least 2, 3, 4, 5, 6, 7, 8, 9, or 10 gigasomes or membrane-bound bodies.

29. The method of any of the preceding embodiments, wherein the method yields at least 1, 10, 100, 500, or 1000 membrane-bound bodies per cell.

30. The method of any of the preceding embodiments, wherein maintenance (e.g., culturing) of the producer cells further comprises adding one or more agents that promote exopheresis.

31. The method of embodiment 30, wherein the one or more agents comprise an autophagy inducer, e.g., trametinib, carbamazepine, or dactolisib, or an mTOR inhibitor (e.g., rapamycin or vistusertib), in an amount sufficient to induce exopheresis by the cell.

32. The method of embodiment 30 or 31, wherein the one or more agents comprise a proteasomal inhibitor, e.g., MG-132 or tripterin, in an amount sufficient to induce exopheresis by the cell.

33. The method of any of embodiments 30-32, wherein the one or more agents comprise an inhibitor of autophagy, e.g., Spautin-1, 3-methyladenine, or SAR405, in an amount sufficient to induce exopheresis by the cell.

34. The method of any of embodiments 30-33, wherein the one or more agents comprise an inhibitor of autophagosome-lysosome fusion, e.g., Bafilomycin-A1, in an amount sufficient to induce exopheresis by the cell.

35. The method of any of embodiments 30-34, wherein the one or more agents comprise an endocytosis inhibitor (e.g., MiTMAB) in an amount sufficient to induce exopheresis by the cell.

36. The method of any of embodiments 30-35, wherein the one or more agents comprise an ER to Golgi inhibitor (e.g., brefeldin A or a monensin, e.g., monensin sodium salt) in an amount sufficient to induce exopheresis by the cell.

37. The method of any of embodiments 30-36, wherein the one or more agents comprise an exocytosis inhibitor (e.g., tipifarnib or simvastatin) in an amount sufficient to induce exopheresis by the cell.

38. The method of any of embodiments 30-37, wherein the one or more agents comprise a proteosomal activator in an amount sufficient to induce exopheresis by the cell.

39. The method of embodiment 38, wherein the proteosomal activator comprises betulinic acid.

40. The method of any of embodiments 30-39, wherein the one or more agents comprise a STAT3 antagonist (e.g., napabucasin) in an amount sufficient to induce exopheresis by the cell.

41. The method of any of embodiments 30-40, wherein the one or more agents comprise a STING antagonist (e.g., H-151) in an amount sufficient to induce exopheresis by the cell.

42. The method of any of embodiments 30-41, wherein the one or more agents comprise a TRAF6 antagonist (e.g., C25-140) in an amount sufficient to induce exopheresis by the cell.

43. The method of any of embodiments 30-42, wherein the one or more agents comprise an iKKb agonist (e.g., betulin) in an amount sufficient to induce exopheresis by the cell.

44. The method of any of embodiments 30-43, wherein the one or more agents comprise an iNOS antagonist (e.g., 1400 W dihydrochloride) in an amount sufficient to induce exopheresis by the cell.

45. The method of any of embodiments 30-44, wherein the one or more agents comprise an LXR antagonist (e.g., GSK2033) in an amount sufficient to induce exopheresis by the cell.

46. The method of any of embodiments 30-45, wherein the one or more agents comprise an LXR agonist (e.g., T0901317) in an amount sufficient to induce exopheresis by the cell.

47. The method of any of embodiments 30-46, wherein the one or more agents comprise an NADPH oxidase antagonist (e.g., apocynin) in an amount sufficient to induce exopheresis by the cell.

48. The method of any of embodiments 30-47, wherein the one or more agents comprise a PPARγ antagonist (e.g., T0070907) in an amount sufficient to induce exopheresis by the cell.

49. The method of any of embodiments 30-48, wherein the one or more agents comprise a glutathione peroxidase inhibitor (e.g., RSL3) in an amount sufficient to induce exopheresis by the cell.

50. The method of any of embodiments 30-49, wherein the one or more agents comprise a stress signaling activator (e.g., anisomycin or SMIP004) in an amount sufficient to induce exopheresis by the cell.

51. The method of any of embodiments 30-50, wherein the one or more agents comprise a cell proliferation inhibitor (e.g., ixabepilone, paclitaxel, or AZD-5438) in an amount sufficient to induce exopheresis by the cell.

52. The method of any of embodiments 30-51, wherein the one or more agents comprise an HDAC inhibitor (e.g., trichostatin A or entinostat) in an amount sufficient to induce exopheresis by the cell.

53. The method of any of embodiments 30-52, wherein the one or more agents comprise a receptor tyrosine kinase inhibitor (e.g. a tyrosine kinase/VEGFR, PDGFR inhibitor) in an amount sufficient to induce exopheresis by the cell.

54. The method of embodiment 53, wherein the tyrosine kinase/VEGFR, PDGFR inhibitor comprises sunitinib.

55. The method any of embodiments 30-54, wherein the one or more agents comprise a SIRT1 inhibitor (e.g., selisistat) in an amount sufficient to induce exopheresis by the cell.

56. The method of any of embodiments 30-55, wherein the one or more agents comprise a SIRT1 activator (e.g., SRT 1720) in an amount sufficient to induce exopheresis by the cell.

57. The method of any of embodiments 30-56, wherein the one or more agents comprise a DNA methyltransferase inhibitor (e.g., 5-azacytidine) in an amount sufficient to induce exopheresis by the cell.

58. The method of any of embodiments 30-57, wherein the one or more agents comprise an AMPA/kainate receptor activator (e.g., diazoxide) in an amount sufficient to induce exopheresis by the cell.

59. The method of any of embodiments 30-58, wherein the one or more agents comprise an AMPA receptor activator, (e.g., CX516) in an amount sufficient to induce exopheresis by the cell.

60. The method of any of embodiments 30-59, wherein the one or more agents comprise an L-type Ca2+ channel activator (e.g., Bay K 8644) in an amount sufficient to induce exopheresis by the cell.

61. The method of any of embodiments 30-60, wherein the one or more agents comprise a CaMK-II inhibitor (e.g., KN-93) in an amount sufficient to induce exopheresis by the cell.

62. The method of any of embodiments 30-61, wherein the one or more agents comprise a CaMK-II activator (e.g., methyl cinnamate) in an amount sufficient to induce exopheresis by the cell.

63. The method of any of embodiments 30-62, wherein the one or more agents comprise an NMDA receptor agonist (e.g., quinolinic acid) in an amount sufficient to induce exopheresis by the cell.

64. The method of any of embodiments 30-63, wherein the one or more agents comprise a NOTCH inhibitor (e.g., DAPT) in an amount sufficient to induce exopheresis by the cell.

65. The method of any of embodiments 30-64, wherein the one or more agents comprise a BACE1 inhibitor (e.g., LY2886721) in an amount sufficient to induce exopheresis by the cell.

66. The method of any of embodiments 30-65, wherein the one or more agents that promote exophoresis are added at a level of 5 nM to 50 nM, 50 nM to 500 nM, 500 nM to 5 μM, 5 μM to 10 μM.

67. The method of embodiment 30-66, wherein the one or more agents are selected from a small molecule (e.g., rapamycin, isoproterenol, hydrogen peroxide, spautin-1, or MG-132, or any combination thereof) and/or an RNAi agent targeting a gene (e.g., wherein the gene is HSF1, ATG7, BECN1, LGG-1/2, UBL5, PINK1, DCT1, PDR1, MTORC1, or AKT, or any combination thereof), and/or a gene editing agent.

68. The method of any of the preceding embodiments, wherein, during the maintaining step, at least 75%, 80%, 85%, 90%, 95%, or 100% of the producer cells are negative for one or more apoptotic signatures, e.g., as measured using a TUNEL assay, Annexin V staining, or caspase levels or activity.

69. The method of any of the preceding embodiments, wherein the producer cells, after the maintaining step, comprise fewer nonessential products (e.g., organelles (e.g., mitochondria, e.g., dysfunctional mitochondria, or lysosomes), protein aggregates, and/or lipids) than before the maintaining the step.

70. The method of any of the preceding embodiments, wherein enriching comprises increasing the concentration of membrane-bound bodies having a diameter of 1-20 μm (e.g., membrane-bound bodies as described herein) by at least 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000-fold.

71. The method of any of the preceding embodiments, wherein the method further comprises loading a cargo into one or more membrane-bound bodies in the preparation.

72. A purified preparation of membrane-bound bodies (e.g., gigasomes), produced by the method of any of the preceding embodiments.

73. The purified preparation of embodiment 72, wherein the membrane-bound bodies (e.g., gigasomes) comprise a cargo, e.g., an exogenous cargo.

74. A purified preparation of membrane-bound bodies (e.g., gigasomes), wherein the membrane bound bodies of the preparation:
are about 1-20 μm in diameter,
comprise one or more human protein;
and the membrane bound bodies have one or more of the following characteristics:
a) comprise an organelle (e.g., mitochondria or lysosomes
b) comprise a product nonessential to a producer cell from which the membrane bound bodies are produced (e.g., dysfunctional mitochondria or a protein aggregate);
c) an excitation ratio (405/476 nm) of at least about 1.2, 1.4, or 1.6, or about 1.2-1.8, 1.4-1.8, e.g., as measured using a mitoROGFP oxidation assay, e.g., as described in Melentijevic et al 2017; or
d) are enriched for LC3 and/or phosphatidylserine.

75. The purified preparation of membrane-bound bodies of embodiment 74, wherein the membrane-bound bodies originate from human cells.

76. The purified preparation of membrane-bound bodies of embodiment 75, wherein the human cells comprise neurons (e.g., HCN2 cells, or HT22 cells), neural progenitor cells, muscle cell, (e.g., cardiac muscle cells), stem cells (e.g., induced pluripotent stem cells (iPSC)), endothelial cells (e.g., microvascular endothelial cells, e.g., cerebral microvascular endothelial cells), HBEC-5i, ReNcell CX, or iCell GlutaNeurons.

77. The purified preparation of membrane-bound bodies of embodiment 75, wherein the human cells are primary cells (e.g., neuronal cells, neural progenitor cells, muscle cells (e.g., cardiac muscle cells), endothelial cells, or stem cells).

78. The purified preparation of membrane-bound bodies of any of embodiments 72-77, wherein the membrane-bound bodies or gigasomes comprise a cargo, e.g., an exogenous cargo.

79. A method of modulating dysregulated exopheresis in a cell, the method comprising inducing or inhibiting exopheresis in the cell, e.g., by contacting the cell with an agent that induces or inhibits exopheresis.

80. The method of embodiment 79, wherein inhibiting exopheresis in the cell comprises contacting the cell with (e.g., administering to a mammal comprising the cell) an exocytosis inhibitor (e.g., GW4869), a Post-Golgi Exocytosis Inhibitor (e.g., Exo 1), a N- and P/Q-type Ca2+ channel Agonist (e.g., GV-58), an NMDA Receptor Activator (e.g., NMDA), a NOTCH Inhibitor (e.g., Semagacestat), a BACE1Inhibitor (e.g., Verubecestat), a Stress Signaling Inhibitor (e.g., Neflamapimod), a Stress signaling activator (e.g., SMIP004), a Mitochondrial pyruvate carrier inhibitor (e.g., UK-5099), an autophagy inhibitor (e.g., Hydroxychloroquine), a IL-1B/NLRP3 antagonist (e.g., MCC950), a Sirtuin Activator (e.g., OSS128167), a Cell proliferation Inhibitor (e.g., Seliciclib), or a proteasome activator (e.g., Oleuropein).

81. The method of embodiment 80, wherein inhibiting exopheresis in the cell comprises contacting the cell with (e.g., administering to a mammal comprising the cell) an iKKb antagonist (e.g., wedelolactone), an iNOS antagonist (e.g., 1400 W dihydrochloride), a histamine H2 receptor agonist (e.g., dimaprit dihydrochloride), GLUT1 inhibitor (e.g., WZB117), a succinate dehydrogenase inhibitor (e.g., 3-nitropropanoic acid), a mitochondrial Na+/Ca2+ exchanger inhibitor (e.g., CGP37157), an acetyl-CoA carboxylase inhibitor (e.g., TOFA), a stress signaling inhibitor (e.g., neflamapimod), an autophagy inhibitor (e.g., 3-methyladenine or spautin-1), or a L-type CA2+ channel activator (e.g., Bay K 8644).

82. A method of improving the health or function of a cell in a mammalian subject (e.g., human subject), the method comprising inducing exopheresis by the cell, e.g., by contacting the cell with one or more agents that induce exopheresis.

83. The method of embodiment 79 or 82, wherein the exopheresis reduces the quantity and/or concentration of a nonessential product in the cell.

84. The method of embodiment 83, wherein the nonessential product comprises a protein aggregate or dysfunctional mitochondria.

85. The method of any of embodiments 82-84, which comprises administering to the mammalian subject an autophagy inducer, e.g., trametinib, carbamazepine, or dactolisib, or an mTOR inhibitor (e.g., rapamycin or vistusertib), in an amount sufficient to induce exopheresis by the cell.

86. The method of any of embodiments 82-85, which comprises administering to the mammalian subject a proteasomal inhibitor, e.g., MG-132 or tripterin, in an amount sufficient to induce exopheresis by the cell.

87. The method of any of embodiments 82-86, which comprises administering to the mammalian subject an inhibitor of autophagy, e.g., Spautin-1, 3-methyladenine, or SAR405, in an amount sufficient to induce exopheresis by the cell.

88. The method of any of embodiments 82-87, which comprises administering to the mammalian subject an inhibitor of autophagosome-lysosome fusion, e.g., Bafilomycin-A1, in an amount sufficient to induce exopheresis by the cell.

89. The method of any of embodiments 82-88, which comprises administering to the mammalian subject an endocytosis inhibitor (e.g., MiTMAB) in an amount sufficient to induce exopheresis by the cell.

90. The method of any of embodiments 82-89, which comprises administering to the mammalian subject an ER to Golgi inhibitor (e.g., brefeldin A or monensin sodium salt) in an amount sufficient to induce exopheresis by the cell.

91. The method of any of embodiments 82-90, which comprises administering to the mammalian subject an exocytosis inhibitor (e.g., tipifarnib or simvastatin) in an amount sufficient to induce exopheresis by the cell.

92. The method of any of embodiments 82-91, which comprises administering to the mammalian subject a proteosomal activator in an amount sufficient to induce exopheresis by the cell.

93. The method of embodiment 92, wherein the proteosomal activator comprises betulinic acid.

94. The method of any of embodiments 82-93, which comprises administering to the mammalian subject a STAT3 antagonist (e.g., napabucasin) in an amount sufficient to induce exopheresis by the cell.

95. The method of any of embodiments 82-94, which comprises administering to the mammalian subject a STING antagonist (e.g., H-151) in an amount sufficient to induce exopheresis by the cell.

96. The method of any of embodiments 82-95, which comprises administering to the mammalian subject a TRAF6 antagonist (e.g., C25-140) in an amount sufficient to induce exopheresis by the cell.

97. The method of any of embodiments 82-96, which comprises administering to the mammalian subject an iKKb agonist (e.g., betulin) in an amount sufficient to induce exopheresis by the cell.

98. The method of any of embodiments 82-97, which comprises administering to the mammalian subject an iNOS antagonist (e.g., 1400 W dihydrochloride) in an amount sufficient to induce exopheresis by the cell.

99. The method of any of embodiments 82-98, which comprises administering to the mammalian subject an LXR antagonist (e.g., GSK2033) in an amount sufficient to induce exopheresis by the cell.

100. The method of any of embodiments 82-99, which comprises administering to the mammalian subject an LXR agonist (e.g., T0901317) in an amount sufficient to induce exopheresis by the cell.

101. The method of any of embodiments 82-100, which comprises administering to the mammalian subject an NADPH oxidase antagonist (e.g., apocynin) in an amount sufficient to induce exopheresis by the cell.

102. The method of any of embodiments 82-101, which comprises administering to the mammalian subject a PPARγ antagonist (e.g., T0070907) in an amount sufficient to induce exopheresis by the cell.

103. The method of any of embodiments 82-102, which comprises administering to the mammalian subject a glutathione peroxidase inhibitor (e.g., RSL3) in an amount sufficient to induce exopheresis by the cell.

104. The method of any of embodiments 82-103, which comprises administering to the mammalian subject a stress signaling activator (e.g., anisomycin or SMIP004) in an amount sufficient to induce exopheresis by the cell.

105. The method of any of embodiments 82-104, which comprises administering to the mammalian subject a cell proliferation inhibitor (e.g., ixabepilone, paclitaxel, or AZD-5438) in an amount sufficient to induce exopheresis by the cell.

106. The method of any of embodiments 82-105, which comprises administering to the mammalian subject an HDAC inhibitor (e.g., trichostatin A or entinostat) in an amount sufficient to induce exopheresis by the cell.

107. The method of any of embodiments 82-106, which comprises administering to the mammalian subject a receptor tyrosine kinase inhibitor (e.g. a tyrosine kinase/VEGFR, PDGFR inhibitor) in an amount sufficient to induce exopheresis by the cell.

108. The method of embodiment 107, wherein the tyrosine kinase/VEGFR, PDGFR inhibitor comprises sunitinib.

109. The method of any of embodiments 82-108, which comprises administering to the mammalian subject a SIRT1 inhibitor (e.g., selisistat) in an amount sufficient to induce exopheresis by the cell.

110. The method of any of embodiments 82-109, which comprises administering to the mammalian subject a SIRT1 activator (e.g., SRT 1720) in an amount sufficient to induce exopheresis by the cell.

111. The method of any of embodiments 82-110, which comprises administering to the mammalian subject a DNA methyltransferase inhibitor (e.g., 5-azacytidine) in an amount sufficient to induce exopheresis by the cell.

112. The method of any of embodiments 82-111, which comprises administering to the mammalian subject an AMPA/kainate receptor activator (e.g., diazoxide) in an amount sufficient to induce exopheresis by the cell.

113. The method of any of embodiments 82-112, which comprises administering to the mammalian subject an AMPA receptor activator, (e.g., CX516) in an amount sufficient to induce exopheresis by the cell.

114. The method of any of embodiments 82-113, which comprises administering to the mammalian subject an L-type Ca2+ channel activator (e.g., Bay K 8644) in an amount sufficient to induce exopheresis by the cell.

115. The method of any of embodiments 82-114, which comprises administering to the mammalian subject a CaMK-II inhibitor (e.g., KN-93) in an amount sufficient to induce exopheresis by the cell.

116. The method of any of embodiments 82-115, which comprises administering to the mammalian subject a CaMK-II activator (e.g., methyl cinnamate) in an amount sufficient to induce exopheresis by the cell.

117. The method of any of embodiments 82-116, which comprises administering to the mammalian subject an NMDA receptor agonist (e.g., quinolinic acid) in an amount sufficient to induce exopheresis by the cell.

118. The method of any of embodiments 82-117, which comprises administering to the mammalian subject a NOTCH inhibitor (e.g., DAPT) in an amount sufficient to induce exopheresis by the cell.

119. The method of any of embodiments 82-118, which comprises administering to the mammalian subject a BACE1 inhibitor (e.g., LY2886721) in an amount sufficient to induce exopheresis by the cell.

120. The method of any of embodiments 82-119, which comprises administering to the mammal two agents that induces exopheresis.

121. A method of delivering a cargo to a target cell, the method comprising contacting the target cell with a purified preparation of any of embodiments 73-78 under conditions suitable for delivery of the cargo to the target cell.

122. A method of delivering cargo to a target cell, the method comprising:
providing a gigasome preparation, wherein gigasomes of the preparation comprise cargo, and
contacting the target cell with the gigasome preparation, thereby delivering the cargo to the target cell.

123. A method of delivering membrane-bound bodies or gigasomes to a target cell, the method comprising contacting the target cell with a purified preparation of any of embodiments 72-78 under conditions suitable for delivery of the membrane-bound bodies or gigasomes to the target cell.

124. A method of delivering a gigasome to a human target cell, the method comprising contacting the human target cell with a gigasome preparation, thereby delivering a gigasome to the target cell.

125. A method of modulating the inflammatory state of a target cell, the method comprising contacting the target cell with a gigasome or membrane-bound body (e.g., a gigasome or membrane-bound body derived from a human cell), thereby modulating the inflammatory state of the cell.

126. The method of embodiment 125, wherein the target cell comprises a macrophage.

127. The method of embodiment 125 or 126, wherein modulating the inflammatory state of the target cell comprises upregulating (e.g., upregulation in one or both of cytokine expression or cytokine secretion) one or more markers of inflammation, wherein optionally the one or more upregulated markers comprise IL-6 or a marker of Table 25.

128. The method of embodiment 125 or 126, wherein modulating the inflammatory state of the target cell comprises downregulating (e.g., downregulation in one or both of cytokine expression or cytokine secretion) one or more markers of inflammation, wherein optionally the one or more downregulated markers comprise IL1-beta, TNF-alpha, or IL-8.

129. The method of any of embodiments 125-128, wherein the target cell is in a subject or is ex vivo.

130. The method of embodiment 129, which further comprises assaying inflammation in the subject, e.g., before or after contacting the target cell with the gigasome or membrane-bound body.

131. The method of embodiment 129, which further comprises assaying a marker of inflammation (e.g., a marker of inflammation disclosed herein) in the subject or ex vivo cell, e.g., before or after contacting the target cell with the gigasome or membrane-bound body.

132. The method of any of embodiments 125-131, wherein the gigasomes or membrane-bound bodies are preferentially taken up by macrophages.

133. The method of any of embodiments 125-132, wherein the gigasome or membrane-bound body does not comprise an exogenous cargo.

134. The method of any of embodiments 125-132, wherein the gigasome or membrane-bound body comprises an exogenous cargo.

135. The method of embodiment 129, wherein the target cell is ex vivo, and wherein the method further comprises administering the target cell to a subject.

136. The method of any of embodiments 121-135, wherein delivery to the target cell comprises phagocytosis of the membrane-bound bodies by the target cell.

137. The method of any one of embodiments 121-136, wherein the target cell is situated in a subject, and the method comprises administering the exopher, gigasome, or membrane-bound body to the subject.

138. The method of embodiment 137, wherein the exopher, gigasome, or membrane-bound body is allogeneic or autologous to the subject.

139. The method of any of embodiments 121-138, wherein the exopher or gigasome is an exopher or gigasome according to any of the preceding embodiments.

140. The method of any of embodiments 121-139, wherein the exopher or gigasome was produced in vitro by a producer cell.

141. The method of embodiment 140, wherein the cargo is exogenous to the producer cell.

142. The method or purified preparation of any of the preceding embodiments, wherein the gigasome, exopher, or membrane-bound preparation comprises a level, normalized to a housekeeping protein value, of a protein of Table 17 that is greater than a level of said protein in column 7 of Table 17.

143. The method or purified preparation of embodiment 142, wherein the level of said protein is at least 2, 3, or 4-fold above the level of said protein in column 7 of Table 17.

144. The method or purified preparation of any of the preceding embodiments, wherein the gigasome, exopher, or membrane-bound preparation comprises a level, normalized to a housekeeping protein value, of a protein of Table 17 that is greater than or equal to a level of said protein in column 6 of Table 17.

145. The method or purified preparation of embodiment 144, wherein the level of said protein is at least 70%, 80% or 90% of the level of said protein in column 6 of Table 17.

146. The method or purified preparation of any of the preceding embodiments, wherein the gigasome, exopher, or membrane-bound preparation comprises a level of a protein, normalized to a housekeeping protein value, of Table 17 that is greater than or equal to a level of said protein in column 5 of Table 17.

147. The method or purified preparation of embodiment 146, wherein the level of said protein is at least 70%, 80% or 90% of the level of said protein in column 5 of Table 17.

148. The method or purified preparation of any of the preceding embodiments, wherein the gigasome, exopher, or membrane-bound preparation comprises a level of a protein, normalized to a housekeeping protein value, of Table 17 that is greater than or equal to a level of said protein in column 4 of Table 17.

149. The method or purified preparation of embodiment 148, wherein the level of said protein is at least 70%, 80% or 90% of the level of said protein in column 4 of Table 17.

150. The method or purified preparation of any of the preceding embodiments, wherein the gigasome, exopher, or membrane-bound preparation comprises a higher level (e.g., a log 2 fold change greater than 2.5, 3, 3.5, 4, or 4.5) of a protein of Table 16 or 17 compared to an apoptotic body or preparation of apoptotic bodies, wherein the apoptotic body or preparation of apoptotic bodies was produced by a method comprising treating a cell with 500 nM Staurosporine, wherein the cell is of the same type of the cell used to produce the gigasome, exopher, or membrane-bound preparation, e.g., as described in Example 16.

151. The method or purified preparation of any of the preceding embodiments, wherein the gigasome, exopher, or membrane-bound preparation comprises a level, normalized to a housekeeping protein value, of a protein of Table 19 that is below a level of said protein in column 7 of Table 19, e.g., lower by 10%, 20%, 30%, 40%, 50%, 60%, or 70%.

152. The method or purified preparation of any of the preceding embodiments, wherein the gigasome, exopher, or membrane-bound preparation comprises a lower level (e.g., by a log 2 fold change having a greater magnitude than 0.5, 1, 1.5, or 2) of a protein of Table 18 or 19 compared to an apoptotic body or preparation of apoptotic bodies, wherein the apoptotic body or preparation of apoptotic bodies was produced by a method comprising treating a cell with 500 nM Staurosporine, wherein the cell is of the same type of the cell used to produce the gigasome, exopher, or membrane-bound preparation, e.g., as described in Example 16.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF DRAWINGS

FIG. 1A is a series of microscopy images taken at multiple time points of a gigasome produced from parent neuronal cell. The gigasome is indicated and tracked with arrows. The gigasome is 5 μm in diameter and has distinct neuronal cytoplasmic and mitochondrial fluorescent signal, with no nuclear signal. The gigasome was produced and separated from the parent cell between 3-7 hours after beginning incubation with MG-132. Timestamp of hours since incubation with MG-132 treatment shown top left of each image.

FIG. 1B is a series of images of a parent neuronal cell in the process of producing a gigasome. The gigasome contains cytoplasmic content and mitochondria but no nuclear content.

FIG. 2A is a series of confocal images of a neuroblastoma cell producing a gigasome co-stained with cytosolic, mitochondrial, and nuclear markers, following MG-132 treatment. A gigasome (arrow) in the proximity of a cell is characterized by neuronal cytoplasmic and mitochondrial fluorescent signals and absence of nuclear marker signal. Scale bar—5 μm.

FIG. 2B is a graph showing quantification of mitochondrial and nuclei content within the parent cells and gigasomes produced by neuroblastoma cells, following MG-132 treatment.

FIG. 2C is a series of confocal images of a neuroblastoma cell producing a gigasome co-stained with cytosolic, lysosomal, and nuclear markers, following MG-132 treatment. A gigasome (arrow) in the process of being produced by a cell (dashed lines) is characterized by a neuronal cytoplasmic and lysosomal fluorescent signal, with no nuclear signal. Scale bar—5 μm.

FIG. 2D is a graph showing quantification of the lysosomal and nuclei content within the parent cells and gigasomes produced by neuroblastoma cells following MG-132 treatment.

FIG. 3A is a series of confocal images of a cardiomyocyte cell co-stained with cytosolic, mitochondrial, and nuclear markers, following rapamycin treatment. A gigasome (arrow) in the proximity of a cell is characterized by cytoplasmic and mitochondrial fluorescent signals and absence of nuclear marker signal. Scale bar—5 μm.

FIG. 3B is a graph showing quantification of mitochondrial and nuclear signal in the cells as compared to the gigasomes produced by cardiomyocytes, following rapamycin treatment.

FIG. 3C is a series of confocal images of a cardiomyocyte cell co-stained with cytosolic, lysosomal, and nuclear markers, following rapamycin treatment. A gigasome (arrow) in the proximity of a cell (dashed lines) is characterized by a cytoplasmic and lysosomal fluorescent signal, with no nuclear signal. Scale bar—5 μm.

FIG. 3D is a graph showing quantification of mean intensity of the lysosomal and nuclear signal in the cells as compared to the gigasomes produced by cardiomyocytes, following rapamycin treatment.

FIG. 4A-4L are graphs showing quantification and characterization of the size (FIGS. 4A, 4E, and 4I), circularity (FIGS. 4B, 4F, and 4J), cytosolic intensity (FIG. 4C, G, K), and mitochondrial intensity (FIGS. 4D, 4H, and 4L) of gigasomes enriched from cell culture media from neuronal cells treated with compounds as indicated.

FIG. 5A is a series of confocal images of neuroblastoma cells treated with γ-secretase inhibitor (5 μM) in the absence or presence of MG-132 (0.1 μM) for 24 h. Treatment with γ-secretase inhibitor increases the intracellular fluorescent signal of APP-CTFs, as detected by an antibody against the C-terminus of APP (C-APP). MG-132 alone does not increase intracellular APP-CTFs. In all treatment groups, except for DMSO, C-APP signal can be seen within gigasomes (arrows) characterized by neuronal cytoplasmic fluorescence and absence of nuclear marker signal. Scale bar—20 μm.

FIG. 5B is a series of images with details of C-APP+ve gigasome shown in FIG. 5A. Scale bar—5 μm.

FIG. 6A-FIG. 6D is a series of graphs showing quantification and characterization of cellular and gigasome-associated APP-CTF signal from IF confocal images. FIG. 6A is a graph showing intracellular APP-CTF levels in the different treatment group expressed as mean C-APP fluorescence intensity/nuclei. Each bar indicates the number of cells evaluated from different confocal images. From left to right, the values represented by the bars are: DMSO, 179 cells; γ-secretase inhibitor, 212 cells; MG-132, 174 cells; γ-secretase inhibitor+MG-132, 156 cells. FIG. 6B is a graph showing quantification of particle counts per 1000 cells. Black bars represent the number of particles containing APP-CTF as detected by C-APP (C-APP+ve). The percentage of C-APP+ve gigasomes with respect to the total gigasomes are indicated. Gray bars represent C-APP negative (C-APP−ve) particles. FIG. 6C is a graph showing mean C-APP particle intensity in the different treatment groups. FIG. 6D is a graph showing mean C-APP intensity in relation to particle size distribution in the different treatment groups.

FIG. 7A is a series of confocal images of gigasomes generated by neuroblastoma cells treated with sodium arsenite (5 μM) in the absence or presence of MG-132 (0.2 μM) for 24 h. Stress granule-associated fluorescent signal within gigasomes (arrows) was detected using an antibody against the RNA binding protein HuR. Scale bar—5 μm.

FIG. 7B-FIG. 7E is a series of graphs showing quantification and characterization of cellular and gigasome-associated HuR signal from IF confocal images. FIG. 7B is a graph showing cytosolic HuR levels, expressed as mean cytosolic HuR intensity/nuclei, in the different treatment groups. Each bar indicates the number of cells evaluated from different confocal images. FIG. 7C is a graph showing quantification of particles per 1000 cells. Black bars represent the number of particles containing HuR (HuR+ve); gray bars represent HuR negative (HuR−ve) particles. FIG. 7D is a graph showing mean HuR particle intensity in the different treatment groups. FIG. 7E is a graph showing mean HuR intensity in relation to particle size distribution in the different treatment groups.

FIG. 8A is a series of confocal images of gigasomes generated by neuroblastoma cells and media counts, following uptake of Alexa-488-labeled Tau fibrils (50 nM) in the absence or presence of post-treatment with MG-132 (0.1 μM) for 24 h. Intracellular fluorescent signal associated with Tau-fibrils in live cell (end time point) was detected only in Tau-F-group, and not in DMSO nor MG-132 groups. Cytosolic marker-positive, nuclear marker-negative gigasomes (arrows) were detected in all treatment groups. Only Tau-F group generated gigasomes containing Tau fibrils. Scale bar—10 μm.

FIG. 8B is a graph showing quantification of cellular Tau fibril (Tau-F) immunofluorescence in the different treatment groups.

FIG. 8C is a graph showing quantification of particles per 1000 cells. Black bars represent the number of particles containing Tau fibrils (Tau+ve); gray bars represent Tau fibril negative (Tau−ve) particles.

FIG. 8D is a series of representative confocal images of gigasomes isolated from neuronal cell culture media. Scale bar—10 μm.

FIG. 8E is a graph showing quantification of gigasomes isolated from neuronal cell culture media. Black bars represent the number of particles containing Tau fibrils (Tau+ve); gray bars represent Tau fibril negative (Tau−ve) particles.

FIG. 9 is a graph showing quantification of the percent of neuronal gigasomes coming from cells that were treated with a combination of MG-132 and Bafilomycin-A1 that contain specified organelles based on analysis of fluorescence microscopy images.

FIG. 10A-10P is a series of graphs showing quantification and characterization of the size, circularity, cytoplasmic and mitochondrial intensity of gigasomes enriched from cell culture media from cardiomyocyte cells treated with compounds as indicated.

FIG. 11 is a principal component analysis (PCA) plot of apoptotic bodies, gigasomes enriched from cells treated with 10 nM MG-132+31 nM Bafilomycin A1 (referred to as Gigasome Group A); gigasomes enriched from cells treated with 100 nM MG-132 (referred to as Gigasome Group B); and gigasomes enriched from cells treated with 31 nM Bafilomycin A1 (referred to as Gigasome Group C).

FIG. 12 is a graph depicting average log 2 values of the raw counts of housekeeping proteins in Gigasomes Group A, B, C and apoptotic bodies.

FIG. 13A-C is a series of images showing macrophages phagocytosing gigasomes. In FIGS. 13A and 13B, Celltracker-Green labeled gigasomes (arrows in top panel, time=0 hours) can be observed near macrophages (ESID Channel/left columns). Over the course of several hours (t=4-5 h), macrophages bound and phagocytosed gigasomes, resulting in digestion and loss of fluorescence. In FIG. 13C, gigasomes were labeled with Celltracker green and pHrodo—a dye that is pH-sensitive and only fluorescent upon macrophage phagocytosis and delivery to lysosomes. Gigasomes not in contact with macrophages or in early phases of digestion can be observed with remaining Celltracker green (arrow), whereas Celltracker green was at least partially lost and pHrodo was active as gigasomes were phagocytosed by macrophages (arrows).

FIG. 14A-B is a series of graphs showing that exogenously applied gigasomes modulate basal and LPS-induced cytokine secretion in THP-1 macrophages differently from apoptotic bodies or cells. For gigasomes, apoptotic bodies, and apoptotic cells the number following (e.g., 10) indicates the number of particles added per target macrophage. FIG. 14A depicts levels of cytokines present in THP-1 macrophage supernatants left untreated or treated with the respective doses of gigasomes. FIG. 14B depicts levels of cytokines present in THP-1 macrophage supernatants treated with LPS with or without the indicated gigasomes.

FIG. 15A-E is a series of graphs showing that exogenously applied gigasomes dose-dependently modulate LPS-stimulated proinflammatory cytokine secretion in a manner distinct from apoptotic bodies. For gigasomes and apoptotic bodies the number following (e.g., 2, 4, or 8) indicates the number of particles added per target macrophage. Cytokine levels including IL-1β (FIG. 15A), IL-10 (FIG. 15B), TNF-α (FIG. 15C), GM-CSF (FIG. 15D), or IL-6 (FIG. 15E) were measured in macrophage supernatants treated with LPS and left untreated, or treated with the indicated doses of either Gigasomes or Apoptotic Bodies.

### DETAILED DESCRIPTION OF CERTAIN EMBODIMENTS

### Definitions

The present invention will be described with respect to particular embodiments and with reference to certain figures but the invention is not limited thereto but only by the claims. Terms as set forth hereinafter are generally to be understood in their common sense unless indicated otherwise.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

If hereinafter examples of a term, value, number, etc. are provided in parentheses, this is to be understood as an indication that the examples mentioned in the parentheses can constitute an embodiment.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

As used herein, a nucleic acid “encoding” refers to a nucleic acid sequence encoding an amino acid sequence or a functional polynucleotide (e.g., a non-coding RNA, e.g., an siRNA or miRNA).

As used herein, “enriched” or “enriching” refers to increasing the amount or concentration of a first substance relative to a second substance in a volume. In some instances, the concentration of the first substance is increased. In some instances, the amount or concentration of the second substance is decreased (e.g., while the amount or concentration of the first substance is kept constant). In some instances, the first or second substance is a molecule, a complex of molecules, or an aggregate of molecules (e.g., a protein aggregate). In some instances, the first or second substance is an organelle (e.g., mitochondrion). In some instances, the volume is held constant during the enrichment. In some instances, the volume changes (e.g., increases or decreases) during enrichment. In some instances, enrichment comprises purifying or isolating the first substance. In some instances, a detectable amount of the second substance remains in the volume. In some instances, enrichment comprises selecting the first substance over the second substance, e.g., based on the presence and/or elevated level of a desired characteristic or the absence and/or reduced level of an undesired characteristic, e.g., as described herein.

An “exogenous” agent (e.g., an effector, a nucleic acid (e.g., RNA), a gene, payload, protein) as used herein refers to an agent that is either not comprised by, or not encoded by, a corresponding wild-type cell. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the cell. In some embodiments, the exogenous agent exists naturally in the cell, but is not present at a desired level or at a desired time.

As used here, the term “exophers” refers to naturally occurring large vesicles released into the extracellular space by certain neurons in C. elegans and by cardiac cells in mice, which vesicles contain cytoplasm and are hypothesized to be involved in cellular homeostasis in the organisms in which they've been studied. Exophers have been described in C. elegans and mice.

“Exopheresis”, as described herein, refers to the process of a cell producing a membrane-bound body of about 1-20 m in diameter, which does not comprise a nucleus, for example producing a plurality of such membrane-bound bodies. In some embodiments, exopheresis occurs naturally (e.g., in an organism), and produces exophers. In some embodiments, exopheresis is induced artificially (e.g., in cell culture), and produces gigasomes.

A “gigasome”, as used herein, refers to a non-naturally occurring membrane-bound body of about 1-20 μm in diameter, which does not comprise a nucleus, and produced by a process comprising inducing one or more producer cells to release the membrane-bound bodies, e.g., through a process related to or similar to exopheresis. Typically, the producer cell is viable for a substantial amount of time after release of the gigasome. Typically, the producer cell produces a plurality of gigasomes over a period of time. In some embodiments a gigasome comprises a higher concentration of one or more products nonessential to the producer cell (e.g., organelles, protein aggregates, lipids, carbohydrates, or small molecules) compared to the producer cell. In some embodiments a gigasome comprises a higher concentration of one or more organelles (e.g., mitochondria, lysosomes, endoplasmic reticulum, Golgi apparatus) compared to the producer cell. In some instances, release comprises expulsion, extrusion, budding, or jettisoning of the membrane-bound body from the producer cell. A gigasome is not released by subjecting a cell to shear stress.

A “gigasome preparation”, as used herein, refers to a non-naturally occurring composition comprising a plurality of gigasomes. In some embodiments, the gigasome preparation is made by a process comprising enriching, selecting, or purifying a nuclear membrane-bound bodies of about 1-20 μm in diameter from a cell culture. In some embodiments, the preparation further comprises cell fragments or cells. In some embodiments, the cell fragments or cells are below detectable levels. In some embodiments, the preparation comprises a buffer, salt, antibiotic, or anti-fungal agent.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together. In some embodiments, a heterologous nucleic acid sequence may be present in the same nucleic acid as a naturally occurring nucleic acid sequence.

As used herein, a “housekeeping protein value” refers to the average raw counts of the following proteins: TOMM70 (Mitochondrial import receptor subunit 70), MRPS18A (39S ribosomal protein S18a), POLR2C (DNA-directed RNA polymerase II subunit RPB3), GAPDH (Glyceraldehyde-3-phosphate dehydrogenase) and NDUFB4 (NADH dehydrogenase 1 beta subcomplex subunit 4).

As used herein, “maintaining” cells under certain conditions refers to keeping the cells in those conditions, wherein the cells are at least 50%, 60%, 70%, 80%, 90%, 95% viable, and optionally the cells undergo cell division. In some embodiments, the cells are actively dividing, and in some embodiments, the cells are quiescent. In some embodiments, the conditions are conditions that allow for exopheresis.

As used herein, a “nonessential” product refers to a substance an amount of which can be removed from a parent cell or producer cell without rendering the parent cell or producer cell nonviable. In some instances, a nonessential product is present in excess in the cell. In some instances, a nonessential product can be excreted from the cell without rendering the parent cell or producer cell nonviable. In some instances, a cell is capable of undergoing cell division after removal of the nonessential product from the cell. In some instances, after removal of the nonessential product from the cell, the cell is capable of performing a normal function (e.g., metabolism or division) at a rate or level of at least 75%, 80%, 85%, 90%, 95%, 100% relative to an otherwise identical cell prior to removal of the nonessential product therefrom. In some instances, the nonessential product is endogenous to the parent cell or producer cell.

As used herein, the term “parent cell” refers to a cell in vivo, that produces or is capable of producing exophers through exopheresis.

As used herein, the term “producer cell” refers to a cell ex vivo, e.g., a cell in culture, that is induced to produce, is producing, or is capable of producing gigasomes or membrane-bound bodies having diameters between about 1-20 μm. In some instances, the producer cell has produced, is producing, or is capable of producing at least one gigasome or a plurality of gigasomes, e.g., at least two, three, four, five, six, seven, eight, nine, or ten gigasomes. In some instances, a producer cell releases nonessential products into a plurality of gigasomes it produces.

As used herein, “viable,” when used with respect to a cell, refers to a non-apoptotic and non-necrotic cell having active metabolic functions. A viable cell may be quiescent or dividing. In some instances, a cell's viability is reduced by increasing its propensity to undergo cell death (e.g., apoptosis, necrosis, or autophagy). A “nonviable” cell, as used herein, refers to a cell other than a viable cell.

### TABLE OF CONTENTS

1. Gigasomes
   1. Characteristics
   2. Content Cargo
2. Nonessential Products
   1. Organelles
   2. Proteins
   3. Nucleic Acids
   4. Lipids
   5. Carbohydrates
   6. Small molecules
3. Methods of Manufacturing
   1. Cell culture
   2. Producer cells
   3. Exophoresis Conditions for Exophoresis
   4. Methods of Enrichment/Purification
   5. Quality Control
4. Methods of Delivery
   1. Diseases and Disorders Indications
   2. Pharmaceutical compositions
5. Promoting exopheresis in vivo

### I. Gigasomes

In some aspects, the present disclosure provides, among other things, compositions and methods comprising enriched or purified gigasomes.

Generally, gigasomes are large, a nuclear membrane-bound bodies released from viable producer cells. In some embodiments, the membrane is a lipid bilayer. In some embodiments, a gigasome is about 1 μm-20 μm in diameter. In some embodiments, an gigasome is about 1-5 μm in diameter. In some embodiments, a gigasome is about 5-10 μm in diameter. In some embodiments, a gigasome is about 10-15 μm in diameter. In some embodiments, a gigasome is about 15-20 μm in diameter. In some embodiments, a gigasome is about 3 um-20 um in diameter. In some embodiments, a gigasome is about 4 um-20 um in diameter. It is understood that, in a method of enriching described herein, the diameter need not be assayed directly as part of this method, so long as the procedure is known to enrich for the desired size of membrane-bound bodies (e.g., gigasomes).

In some embodiments, the gigasome comprises phosphatidylserine (PS) in the membrane. In some embodiments, the outer leaflet of the membrane comprises PS. In some embodiments, the gigasome has higher PS concentration in the outer leaflet of the membrane compared to the producer cell membrane. In some embodiments, a gigasome or a gigasome preparation (e.g., as described herein) does not comprise detectable levels of TSPAN4.

In some embodiments, during exopheresis, it takes a producer cell about 15-60 minutes to release a gigasome. In some embodiments, exopheresis comprises outward budding and jettisoning the gigasome from the cell body. In some embodiments, during exopheresis, the gigasome is attached to the producer cell via a thin fiber. In some embodiments, a method described herein is actin-dependent.

In some embodiments, gigasomes comprise cargo. In some embodiments, the cargo comprises material endogenous to the producer cell. In some embodiments, the cargo comprises material exogenous to the producer cell. In some embodiments, the cargo comprises nonessential products to the producer cell. Examples of nonessential products are described below in the section entitled, “Nonessential Products.”

Gigasomes are distinct from exosomes. Exosomes are typically nano-sized extracellular vesicles with a diameter of about 30-160 nm that are released upon fusion of multivesicular bodies and the plasma membrane, using ESCRT machinery.

In some embodiments, a gigasome as described herein is not a migrasome. Migrasomes are vesicles originating from migrating cells. During cell migration, retraction fibers are pulled from the rear end of cells, and migrasomes grow on the retraction fibers. Migrasomes are characterized by the presence of tetraspanin 4 (TSPAN4) (e.g., as described in Ma et al., 2015. Cell Research 25:24-38.).

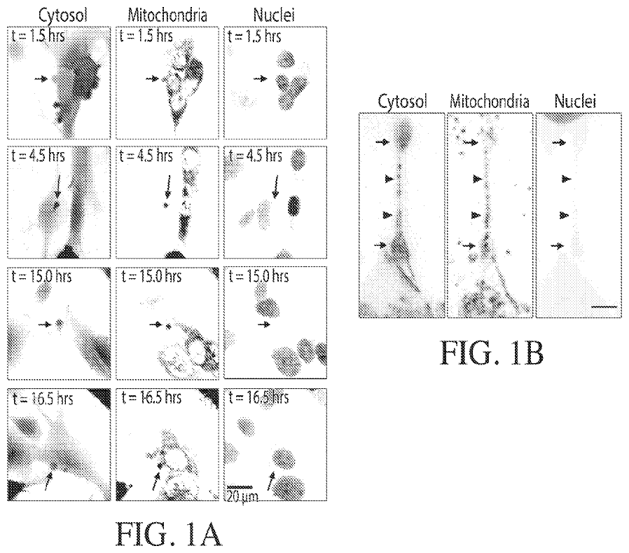

In some embodiments, gigasomes are not oncosomes. In some embodiments, a gigasome is not a large oncosome (e.g., as described in Jeppesen et al., 2019, Cell 177:428-445, which is incorporated by reference in its entirety, including FIG. 1A therein). Large oncosomes are extracellular vesicles derived from cancer cells, which carry oncogenic cargo which promotes oncogenesis or cancer progression. In some embodiments, the transformed cells are cancer or tumor cells.

Gigasomes are distinct from apoptotic bodies. Apoptotic bodies are released by cells undergoing apoptosis and appear after the disassembly of an apoptotic cell into subcellular fragments (e.g., as described in Battistelli et al., 2020. Biology 9(1):21 doi: 10.3390/biology9010021).

Gigasomes are distinct from microvesicles (also called Ectosomes). Microvesicles are typically 50-1,000 nm in diameter and are released from cells using ESCRT machinery (e.g., the ESCRT3 complex and tsg-101).

Gigasomes are distinct from ARMMS (ARRDC1-mediated microvesicles). ARMMS are typically 50-80 nm in diameter and are released from cells using an ARRDC1-mediated mechanism. ARMMS are described in more detail in Wang et al., 2018. Nature Commun 9:960 doi: 10.1038/s41467-018-03390-x, which is herein incorporated by reference in its entirety.

### II. Nonessential Products

In some aspects, the present disclosure provides, among other things, exophers or gigasomes comprising nonessential products from the parent or producer cell, respectively. In some embodiments, the nonessential product is endogenous to the parent cell or producer cell. In some embodiments, the nonessential product is exogenous to the producer cell. In some embodiments, the nonessential product is found in a higher concentration in exophers or gigasomes compared to the parent or producer cell.

In some embodiments, the endogenous nonessential product comprises an organelle. In some embodiments, the endogenous nonessential product comprises a plurality of organelles. In some embodiments, the organelle is a mitochondrion. In some embodiments, the mitochondrion is a normally functioning mitochondrion. In some embodiments, the mitochondrion is a dysfunctional mitochondrion (e.g., having a partial or complete reduction in one or more mitochondrial functions). In some embodiments, the dysfunctional mitochondrion can be characterized by a reduction in a mitochondrial protein, e.g., one or more of Opal, Fis1, Cytc, and Aifm1 (e.g., as described in Nicolas-Avila et al., 2020). In some embodiments, the dysfunctional mitochondrion comprises increased reactive oxygen species (ROS) production. In some embodiments, the dysfunctional mitochondrion comprises low mitochondrial membrane potential. In some embodiments, the dysfunctional mitochondrion comprises mitochondrial DNA (mtDNA) with a deleterious mutation.

In some embodiments, the organelle is a lysosome. In some embodiments, the organelle is a Golgi complex or a portion thereof. In some embodiments, the organelle comprises endoplasmic reticulum or a portion thereof. In some embodiments, a gigasome comprises cytosol or a cytosolic component (e.g., cytoskeleton).

In some embodiments, the nonessential product comprises a protein. In some embodiments, the non-essential product comprises a plurality of proteins within a gigasome. In some embodiments, the protein is endogenous to the producer cell. In some embodiments, the protein is natively encoded in the producer cell. In some embodiments, the protein is exogenous to the producer cell. In some embodiments, the protein is a wild-type protein. In some embodiments, the protein is a mutant protein. In some embodiments, the protein is a misfolded protein. In some embodiments, the protein is an aggregated protein. In some embodiments, the aggregated protein is β-amyloid. In some embodiments, the aggregated protein is tau. In some embodiments, the aggregated protein is huntingtin. In some embodiments, the aggregated protein is desmin. In some embodiments, the protein is a fluorescent protein (e.g., Green Fluorescent Protein (GFP)). In some embodiments, the protein is an aggregation-prone protein (e.g. mCherry as described in Melentijevic et al., 2017. Nature 542:367-373). In some embodiments, the exogenous protein is a therapeutic protein.

In some embodiments, the nonessential product comprises an amino acid. In some embodiments, the amino acid is proteinogenic (e.g., form peptides or proteins). In some embodiments, the amino acid is one of 20 standard amino acids encoded in the genetic code. In some embodiments, the amino acid is a nonessential amino acid (e.g., alanine, aspartic acid, asparagine, glutamic acid, or serine). In some embodiments, the amino acid is an essential amino acid (e.g., phenylalanine, valine, tryptophan, threonine, isoleucine, methionine, histidine, leucine, or lysine). In some embodiments, the amino acid is a nonstandard or non-canonical amino acid (e.g., selenocysteine and pyrrolysine). In some embodiments, the amino acid is non-proteinogenic (e.g, do not form peptides or proteins). In some embodiments, the amino acid is a modified amino acid. In some embodiments, the amino acid is an alpha-(α), beta-(β), gamma-(γ), or delta-(δ) amino acid.

In some embodiments, the nonessential product comprises a nucleic acid molecule. In some embodiments, the nucleic acid molecule is DNA. In some embodiments, the nucleic acid molecule is RNA. In some embodiments, the RNA is a messenger RNA (mRNA). In some embodiments, the RNA is an interfering RNA (RNAi, e.g., microRNA or siRNA). In some embodiments, the RNA is a transfer RNA (tRNA). In some embodiments, the RNA is a ribosomal RNA (rRNA). In some embodiments, the nucleic acid molecule comprises a modified nucleotide.

In some embodiments, the nonessential product comprises a nucleotide. In some embodiments, the nucleotide is a modified nucleotide.

In some embodiments, the nonessential product comprises a lipid. In some embodiments, the lipid is a fatty acid. In some embodiments, the lipid is a sterol (e.g., cholesterol) or a derivative thereof (e.g., steroid hormones). In some embodiments, the lipid is a phospholipid.

In some embodiments, the nonessential product comprises a carbohydrate. In some embodiments, the carbohydrate is a monosaccharide. In some embodiments, the carbohydrate is a disaccharide. In some embodiments, the carbohydrate is an oligosaccharide. In some embodiments, the carbohydrate is a polysaccharide.

In some embodiments, the nonessential product comprises a small molecule, for example an organic compound of <900 daltons.

### III. Methods of Manufacturing

In some aspects, the present disclosure provides methods of making gigasomes and purified preparations comprising gigasomes. Generally, gigasomes are produced by producer cells (e.g., as described herein), by a process in which the producer cell releases one or more gigasomes while maintaining viability. A producer cell may, in some instances, be contacted with and/or incubated under conditions that promoter gigasome production.

### Producer Cells

In some embodiments, the producer cell is a primary cell (e.g., a primary neuron, e.g., as described in Example 1). In some embodiments, the producer cell is from a cell line (e.g., an immortalized cell line). In some embodiments, the producer cell is a cell type as listed in Table 1 or 4. In some embodiments, the producer cell is a neuron (e.g., a neuron derived from an induced pluripotent stem cell). In embodiments, the producer cell is a cortical neuron (e.g., an HCN2 cell), e.g., glutamatergic-enriched cortical neurons (e.g., iCell GlutaNeurons). In some embodiments, the producer cell is a hippocampal neuron (e.g., an HT22 cell). In some embodiments, the producer cell is a neural progenitor cell (e.g., a ReNcell CX cell), e.g., a neuroblast (e.g., an SH-SY5Y cell). In some embodiments, the producer cell is a stem cell. In some embodiments, the producer cell is an induced pluripotent stem cell (iPSC). In some embodiments, the producer cell is an endothelial cell (e.g., an HBEC-5i cell). In some embodiments, the producer cell is a muscle cell.

In some embodiments, the producer cell is a long-lived cell type. For instance, in some embodiments, the producer cell is a cell type that, when in the human body, lives for at least 1, 2, 3, 4, 5, or 10 years.

Typically, the producer cell is viable for a substantial amount of time after release of the gigasome. For example, in some embodiments, the producer cell is viable for 1, 2, 3, 5, or 10 days after release of the gigasome. In some embodiments, the producer cell divides and produces daughter cells that are still viable for 1, 2, 3, 5, or 10 days after release of the gigasome. In some embodiments, the producer cell does not undergo cell death (e.g., apoptosis) for 1, 2, 3, 5, or 10 days after release of the gigasome.

### Promoting or Inducing Gigasome Production

In some embodiments, in a method described herein, the producer cell is contacted with and/or incubated in the presence of a compound that promotes or induces gigasome production. In some embodiments, the compound is a small molecule. In some embodiments, the compound is selected from those listed in Tables 2 or 5. In embodiments, the compound is selected from rapamycin (e.g., as described in Nicolas-Avila et al. 2020. Cell 183:1-16), incorporated herein by reference in its entirety), isoproterenol (e.g., as described in Nicolas-Avila 2020, supra), hydrogen peroxide (e.g., as described in Fu et al. 2019, Biorxiv, incorporated herein by reference in its entirety), spautin-1, MG-132, chloramphenicol (e.g., as described in Tian et al. 2016. Oncotarget 7:51934-51942), bafilomycin (e.g., as described in Redmann et al. 2017. Redox Biol 11:73-81), or carbonyl cyanide 3-chlorophenylhydrazone (CCCP) (e.g., as described in Charan et al. 2014. Cell Death Disease 5:e1313). In some embodiments, the producer cell is contacted with and/or incubated in the presence of the compound at a concentration described herein. In some embodiments, the compound is at a concentration between about 1 nM to about 10 mM. In some embodiments, the concentration is between about 1 nM to about 5 nM, about 5 nM to about 10 nM, about 10 nM to about 100 nM, about 100 to about 500 nM, about 500 nM to about 1 uM, about 1 uM to about 10 uM, about 10 uM to about 100 uM, about 100 uM to about 500 uM, about 500 uM to about 1 mM, about 1 mM to about 5 mM, about 5 mM to about 10 mM. In some embodiments, the producer cell is contacted with and/or incubated in the presence of the compound for a duration described herein. In some embodiments, the producer cell is contacted with and/or incubated in the presence of the compound for up to 48 hours (e.g., between about 30 minutes about about one hour, between one hour and about 5 hours, between about 5 hours and about 10 hours, between about 10 hours and about 15 hours, between about 15 hours and about 20 hours, between about 20 hours and about 25 hours, between about 25 hours and about 30 hours, between about 30 hours and about 35 hours, between 35 hours and about 40 hours, or between about 40 hours and about 48 hours).

In some embodiments, the compound that promotes or induces gigasome production comprises an autophagy inducer, e.g., rapamycin. In some embodiments, the compound that promotes or induces gigasome production comprises a proteasome inhibitor, e.g., MG-132. In some embodiments, the compound that promotes or induces gigasome production comprises an inhibitor of autophagy, e.g., Spautin-1. In some embodiments, the compound that promotes or induces gigasome production comprises an inhibitor of autophagosome-lysosome fusion autophagy inducer, e.g., Bafilomycin-A1. In some embodiments, gigasome production is induced or increased by altering (e.g., increasing or decreasing) the expression or activity of a gene or gene product in the producer cell. In some embodiments, a gene is knocked out of the producer cell's genome. In some embodiments, a gene product is knocked down in the producer cell (e.g., by RNA interference, e.g., using an siRNA targeting an mRNA encoding the gene product). In some embodiments, the gene or gene product is selected from those listed in Tables 3 or 6. In embodiments, the gene or gene product is selected from HSF-1, ATG7, BECN1, IGG-1/2, UBL5, PINK1, DCT1, PDR1, MTORC, and AKT.

In some embodiments, gigasome production is induced or increased by inducing or increasing stress in the producer cell. In some embodiments, the stress is oxidative stress. In some embodiments, the stress is proteotoxic stress. In some embodiments, gigasome production is induced or increased by modulating (e.g., stimulating or inhibiting) autophagy. In some embodiments, autophagy is inhibited. In some embodiments, autophagy is measured by an increase or decrease in LC3. In some embodiments, inducing or increasing stress in the producer cell does not result in apoptosis of the producer cell. In some embodiments, inducing or increasing stress in the producer cell does not result in cell death. In some embodiments, the producer cell remains viable with induced or increased stress.

### Cultures Comprising Producer Cells

In some embodiments, gigasomes are produced from producer cells in monoculture.

In some embodiments, gigasomes are produced from producer cells in co-culture with one or more additional cell types. In some embodiments, the producer cell is co-cultured with microglial cells (e.g., primary microglia or a microglial cell line). In some embodiments, the microglial cell is an embryonic spinal cord microglia, embryonic cortex microglia, embryonic telencephalon microglia, or adult cortex microglia. In some embodiments, the microglial cell is selected from iCell microglia, iCell microglia AD TREM2, CHME-5 cells, HMO6 cells, and Huμglia cells, and HMC3 cells. In some embodiments, the producer cell is directly co-cultured with the one or more additional cell types (e.g., the producer cell and the additional cell types are intermingled or capable of physically contacting each other in the co-culture). In some embodiments, the producer cell is separated from the one or more additional cell types (e.g., via a physical barrier, e.g., a transwell insert or a removable separator). In some embodiments, the producer cell and the one or more additional cell types are co-cultured in an organoid system.

In some embodiments, the producer cell is cultured in suspension. In some embodiments, the producer cell is cultured in adherent culture. In some embodiments, the producer cell is cultured on a plate (e.g., a multi-well plate).

### Enrichment or Isolation of Gigasomes

In some embodiments, a method described herein comprises a step of harvesting, isolating, enriching, or separating the gigasomes from the producer cells. In some embodiments, cell culture media is collected from the producer cell culture. In some embodiments, the cell culture contains gigasomes and one or more of producer cells or cell debris.

In some embodiments, gigasomes are enriched relative to producer cells and/or cell debris. In some embodiments, the cell debris comprises cell fragments. In some embodiments, the cell debris comprises cytosolic content. In some embodiments, the cell debris comprises lipid membranes. In some embodiments, the cell debris comprises membrane-bound bodies other than gigasomes. In some embodiments, the cell debris comprises non-viable producer cells. In some embodiments, the cell culture media is centrifuged. In some embodiments, centrifugation of the cell culture enriches the gigasomes from the producer cells and cell debris.

In some embodiments, gigasomes are enriched or purified by size fractionation, e.g., using size-exclusion chromatography.

In some embodiments, a surface protein on the surface of a producer cell can be used for affinity purification of gigasomes, e.g., in combination with size fractionation.

In some embodiments, gigasomes are enriched or purified by differential ultracentrifugation.

In some embodiments, gigasomes are enriched or purified by floatation in a density barrier or in a density gradient, e.g., in combination with differential ultracentrifugation.

In some embodiments, gigasomes are enriched or purified by density gradient ultracentrifugation.

In some embodiments, gigasomes are enriched or purified by precipitation, e.g., polyethylene glycol (PEG)-based volume exclusion precipitation. In some embodiments, gigasomes are enriched or purified by precipitation as described in Monguió Tortajada et al. (2019, Cellular and Molecular Life Sciences; incorporated herein by reference in its entirety).

In some embodiments, gigasomes are enriched or purified using flow cytometry.

In some embodiments, gigasomes are enriched or purified using ultrafiltration

In some embodiments, gigasomes are enriched or purified using filed-flow fractionation, e.g., according to electrophoretic mobility or hydrodynamic diameter.

### Assessment or Detection of Gigasomes

In some embodiments, the present disclosure provides methods of assessing the quality of gigasomes production and preparation of compositions comprising gigasomes. In some embodiments, gigasome production and compositions comprising gigasomes are characterized using live microscopy. In some embodiments, gigasome production and compositions comprising gigasomes are characterized using immunofluorescent microscopy.

In some embodiments, a gigasome produced by any of the preceding methods is characterized with a detection reagent. In some embodiments, the detection reagent is an antibody. In some embodiments, the antibody is conjugated to a dye. In some embodiments, the antibody is conjugated to a fluorescent protein. In some embodiments, the antibody targets/detects an organelle protein marker. In some embodiments, the antibody targets/detects a cell membrane marker. In some embodiments, the antibody targets/detects a cell-type specific marker.

In some embodiments, the detection reagent is a stain. In some embodiments, the stain detects mitochondria (e.g., MitoTracker). In some embodiments, the stain detects lysosomes (e.g., Lysotracker). In some embodiments, the stain detects nuclei (e.g., Hoechst 33342). In some embodiments, the stain detects cytoplasm (e.g., CellTracker Green). In some embodiments, the stain detects lipid vesicles (e.g., LipidTOX Neutral Lipid Stain). In some embodiments, the stain detects endoplasmic reticulum (e.g., CellLight-ER). In some embodiments, the stain detects Golgi complexes (e.g., CellLight Golgi).

In some embodiments, the present disclosure provides methods of quantification and characterization of gigasome phagocytosis by target cells (e.g., microglia). In some embodiments, the method comprises flow cytometry. In some embodiments, the method comprises live microscopy. In some embodiments, the method comprises immunofluorescent microscopy.

In some embodiments, the producer cells are stained with a fluorescent marker (e.g., CellTracker Green). In some embodiments, the target cells (e.g., microglia) are stained with a fluorescent marker (e.g., CellTracker Red).

In some embodiments, the method of assessment comprises trypsinization of cells from the cell culture dish. In some embodiments, the method comprises centrifugation of the cells. In some embodiments, the method comprises contacting a cell with an antibody. In some embodiments, the antibody targets a cell-type specific marker.

In some embodiments, the method comprises detection of delivery of cargo to target cells by gigasomes. In some embodiments, delivery of cargo to target cells by gigasomes comprise phagocytosis by the target cell. In some embodiments, detection of phagocytosis is assessed using flow cytometry. In some embodiments, detection of phagocytosis is assessed using live microscopy. In some embodiments, detection of phagocytosis is assessed using immunofluorescent microscopy. In some embodiments, detection of phagocytosis is assessed using Western blotting. In some embodiments, detection of phagocytosis is assessed using liquid chromatography in tandem with mass spectrometry (LC-MS).

In some embodiments, the target cell is harvested from the cell culture, e.g., a co-culture as described herein. In some embodiments, the target cell is enriched from the cell culture. In some embodiments, the target cell is labeled with a detection reagent. In some embodiments, the detection reagent is an antibody. In some embodiments, the antibody is a cell-type specific antibody (e.g., anti-CD11b antibody; anti-ACSA2 antibody). In some embodiments, the antibody is conjugated to a stain. In some embodiments, the antibody is conjugated to a fluorescent marker. In some embodiments, the detection reagent is a stain. In some embodiments, the stain is an organelle-specific stain. In some embodiments, the stain is a nucleic acid stain (e.g., Draq5). In some embodiments, the producer cell is stained with one stain (e.g., CellTracker Green) and the target cell is stained with another stain (e.g., CellTracker Red). Phagocytic score is calculated as the percentage of cells taking up producer cell stain over target cell stain×the mean fluorescent intensity/1000. In some embodiments, other descriptive measures (e.g., median, frequency, percent, and/or interquartile range) will be used.

In some embodiments, a gigasome is characterized using flow cytometry. In some embodiments, the gigasome is characterized using confocal microscopy. In some embodiments, the gigasome is characterized using liquid chromatography in tandem with mass spectrometry (LC-MS). In some embodiments, the gigasome is characterized using Western blotting.

In some embodiments, the gigasome is stained for a cell marker. In some embodiments, the stain is an antibody. In some embodiments, the antibody is conjugated to a dye. In some embodiments, the antibody is conjugated to a fluorescent protein. In some embodiments, the antibody targets/detects an organelle protein marker. In some embodiments, the antibody targets/detects a cell membrane marker. In some embodiments, the antibody targets/detects a cell-type specific marker.

In some embodiments, the stain is an organelle-specific stain. In some embodiments, the stain detects mitochondria (e.g., MitoTracker). In some embodiments, the stain detects lysosomes (e.g., Lysotracker). In some embodiments, the stain detects nuclei (e.g., Hoechst 33342). In some embodiments, the stain detects cytoplasm (e.g., CellTracker Green). In some embodiments, the stain detects lipid vesicles (e.g., LipidTOX Neutral Lipid Stain). In some embodiments, the stain detects endoplasmic reticulum (e.g., CellLight-ER). In some embodiments, the stain detects Golgi complexes (e.g., CellLight Golgi).

In some embodiments, the producer cell is characterized during the process of making gigasomes. In some embodiments, the producer cell is characterized using live microscopy. In some embodiments, the producer cell is stained with a detection reagent. In some embodiments, the detection reagent is an antibody. In some embodiments, the antibody is conjugated to a dye. In some embodiments, the antibody is conjugated to a fluorescent protein. In some embodiments, the antibody targets/detects an organelle protein marker. In some embodiments, the antibody targets/detects a cell membrane marker. In some embodiments the antibody targets/detects a cell-type specific marker.

In some embodiments, the detection reagent is a stain. In some embodiments, the stain is an organelle-specific stain. In some embodiments, the stain detects a mitochondrion (e.g., MitoTracker). In some embodiments, the stain detects a lysosome (e.g., Lysotracker). In some embodiments, the stain detects a nucleus (e.g., Hoechst 33342). In some embodiments, the stain detects cytoplasm (e.g., CellTracker Green). In some embodiments, the stain detects a lipid vesicle (e.g., LipidTOX Neutral Lipid Stain). In some embodiments, the stain detects endoplasmic reticulum (e.g., CellLight-ER). In some embodiments, the stain detects a Golgi complex (e.g., CellLight Golgi).

In some embodiments, the producer cell is stained to assess cell viability. In some embodiments, the producer cell is stained with a caspase-specific detection kit (e.g., Image-iT LIVE Red Caspase-3 and -7 Detection Kit, Thermo Fisher Scientific). In some embodiments, the producer is stained for mitochondria (e.g., Mitoview 640). In some embodiments, the producer cells are stained for lysosomes (e.g., CellLight-Lysosomes). In some embodiments, the producer cell is stained for lipid vesicles (e.g., HCS LipidTOX Neutral Lipid Stain). In some embodiments, the producer cell is stained for endoplasmic reticulum (e.g., CellLight-ER). In some embodiments, the producer cell is stained for Golgi complexes (e.g., CellLight Golgi).

### IV. Methods of Delivery and Pharmaceutical Compositions

In some aspects, the present disclosure provides methods and compositions for delivery of gigasomes or cargo comprised within a gigasome to target cells. In some aspects, the present disclosure provides methods and compositions for modulating a biological activity in a target cell, the method comprising contacting the target cell with a gigasome as described herein.

In some embodiments, the target cell is a wild-type cell. In some embodiments, the target cell is a diseased or dysfunctional cell. In some embodiments, the target cell is found in an animal, e.g., a human subject. In some embodiments, the gigasome or cargo is delivered in an amount that is therapeutic to the target cells. In some embodiments, the gigasome is delivered to a target cell in combination with another therapeutic molecule or reagent.

In some embodiments, the target cells are microglia. In some embodiments, the target cell is an embryonic cell. In some embodiments, the target cell is a non-phagocytic cell. In some embodiments, the target cell is a phagocytic cell. In some embodiments, the target cell is not proliferative. In some embodiments, the target cell is not terminally differentiated.

In some embodiments, the disclosure provides a pharmaceutical composition containing an gigasomes or a plurality of gigasomes as described herein, and a pharmaceutically acceptable carrier. In some embodiments, the pharmaceutical composition is sterile. In some embodiments, the pharmaceutical composition is substantially free of macromolecule contaminants. As used herein, the term “macromolecule” means nucleic acids, proteins, lipids, carbohydrates, metabolites, or a combination thereof. As used herein, the term “substantially free” means that the preparation comprises less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules. In some embodiments, the pharmaceutical composition is substantially free of cell debris; substantially free of host cell DNA; substantially free of bacteria; substantially free of viruses; or substantially free of fungi. In some embodiments, the pharmaceutical composition: meets a pharmaceutical or good manufacturing practices (GMP) standard; was made according to good manufacturing practices (GMP); has a pathogen level below a predetermined reference value, e.g., is substantially free of pathogens; has a contaminant level below a predetermined reference value, e.g., is substantially free of contaminants; has a level of membrane bound bodies other than gigasomes that is below a predetermined reference value, e.g., is substantially free of membrane bound bodies other than gigasomes.

While not wishing to be bound by theory, the disclosure contemplates that there are certain disorders by which the spreading or trafficking of a pathological or unwanted material (e.g., viruses, pathological proteins like amyloid, etc.) from one cell to another or from one cell into its microenvironment can further drive the progression or severity of disease. There is evidence to suggest some of these pathological material are capable of trafficking through extracellular vesicles (Yang, et al., 2021, Front. Cell Dev. Biol., Sec. Epigenomics and Epigenetics, 9:722020; Bello-Morales, et al., 2020, Viruses, 12(6):623), and the current ability to control this trafficking is limited. Consequently, the disclosure provides that downregulation of exopher production can limit the spread or trafficking of such pathological or unwanted materials out of diseased cells. In some aspects, the present disclosure provides methods and compositions for inhibiting exopheresis in a cell (e.g., a mammalian cell).

V. Modulating exopheresis in vivo Without wishing to be bound by theory, the disclosure contemplates that the health of a cell may be impaired by high levels of nonessential products, e.g., waste products, in the cell. Consequently, the disclosure provides that inducing exopheresis may improve the health of a cell. In some aspects, the present disclosure provides a method of inducing a mammalian (e.g., human) cell to release one or more exophers. In some embodiments, the cell is in a mammalian subject, e.g., a human subject. In some embodiments, exopheresis is induced by administering to the subject an agent, in an amount sufficient to induce exopheresis. In some embodiments, prior to administration of the agent, the cell underwent exopheresis at a first level, and subsequent to administration of the agent, the cell undergoes exopheresis at a second, higher level. In some embodiments, after administration of the agent, the cell produces 10%, 20%, 30%, 50%, or 100% more exophers per day than the cell prior to administration of the agent. In some embodiments, the agent is an agent described herein, e.g., an agent of Table 2 or 5, or an inhibitor (e.g., siRNA) of a gene of Table 3 or 6. In some embodiments, the exopheresis results in reduced numbers of dysfunctional mitochondria in the cell (e.g., a muscle cell), or reduced amounts of aggregated proteins in the cell (e.g., a neuron).

In some embodiments, the present disclosure provides methods for the production of exophers in vivo. In some embodiments, in vivo production of exophers is induced in an animal. In some embodiments, in vivo production of exophers is induced in a mammal. In some embodiments, the mammal is a mouse. In some embodiments, in vivo production of exophers is induced by injection of a compound that induces exopher production. In some embodiments, the compound is selected from those listed in Table 2 or 5 (e.g., rapamycin). In some embodiments, the compound is injected one a week. In some embodiments, the compound is injected more than once a week. In some embodiments, the compound is injected three times a week.

In some embodiments, tissue is harvested from the animal (e.g., a mammal). In some embodiments, the harvested tissue is separated into cells (e.g., physically or with enzyme (e.g., liberase)). In some embodiments, the cells are separated from the supernatant (e.g., using centrifugation). In some embodiments, the exophers produced in vivo are separated from the supernatant.

The disclosure further contemplates that, in some embodiments, dysregulated exopheresis can negatively impact the health of a cell, and that an agent that returns exopheresis to a more normal level can increase the health of a cell. For instance, in some embodiments, a cell is characterized by abnormally low exopheresis, and the cell can be contacted with an agent that promotes exopheresis. In other embodiments, a cell is characterized by abnormally high exopheresis, and the cell can be contacted with an agent that inhibits exopheresis. The cell may be situated in a subject or ex vivo.

### VI. Gigasomes for Modulating Inflammation

In some embodiments described herein, gigasomes can be used to modulate the inflammatory state of a cell, such as a macrophage. For instance, Example 21 described herein demonstrates that gigasomes can be bound and internalized by macrophages, resulting in modulation of the macrophages. The modulation may comprise, for example, changes in cytokine release, e.g., in a basal state or pro-inflammatory environment. In some embodiments, the modulation comprises upregulation of a gene listed in Table 25 herein. In some embodiments, the modulation comprises an increase in basal levels of IL-6. In some embodiments, the modulation comprises a decrease in basal levels of IL-1β, TNF-α, or IL-8. In some embodiments, the modulation comprises an increase in levels of IL-6 in a proinflammatory environment. In some embodiments, the modulation comprises a decrease in levels of TNF-α in a proinflammatory environment. In some embodiments, the modulation comprises an increase in GM-CSF levels.

In some embodiments, the ratio of gigasomes to target cells (e.g., macrophage) is between 2:1 and 10:1 gigasomes/target cell, for instance between 2:1 and 3:1, 3:1 and 4:1, 4:1 and 5:1, 5:1 and 6:1, 6:1 and 7:1, 7:1 and 8:1, 8:1 and 9:1, or 9:1 and 10:1 gigasomes/target cell.

In some embodiments, a cell (e.g., macrophage) is contacted with gigasomes ex vivo, thereby modulating its inflammatory state. The cell may then be administered to a subject, e.g., as a cell therapy.

In some embodiments, a gigosome preparation is administered to a subject, thereby allowing gigasomes of the preparation to contact macrophages of the subject in vivo. In some embodiments, the gigasomes modulate inflammation in the subject.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Exemplary gigasome production methods
Example 2: Quantification and characterization of neuronal gigasome production
Example 3: Quantification and characterization of neuronal gigasome phagocytosis by microglia
Example 4: In vivo exopher induction in mice
Example 5: Assessment of gigasome production by confocal microscopy
Example 6: Identification and isolation of gigasomes by flow cytometry
Example 7: Biochemistry for detecting transfer of proteins delivered by gigasomes
Example 8: Mass spectrometry
Example 9: Characterization of producer cells in the process of making gigasomes
Example 10: Exemplary gigasome production methods using compounds
Example 11: Visualization of gigasome generation process and characterization of gigasome cargo
Example 12: Modulation and quantification of gigasome yield, purity, and distribution
Example 13: Gigasome-mediated extraction of disease-relevant waste cargo in three neuronal disease models in vitro
Example 14: Visualization gigasome generation process and characterization of gigasome cargo
Example 15: Modulation and quantification of cardiomyocyte gigasome yield, purity, and distribution
Example 16: Proteomic analyses of the gigasomes using mass spectrometry (MS)
Example 17: Small molecule compound screen to reveal up-regulators of gigasome production in neuronal monocultures
Example 18: Small molecule compound screen to reveal down-regulators of gigasome production in neuronal monocultures
Example 19: Small molecule compound screen to reveal up-regulators of gigasome production in cardiomyocytic monocultures
Example 20: Small molecule compound screen to reveal down-regulators of gigasome production in cardiomyocytic monocultures
Example 21: Modulation of macrophages by exogenously applied gigasomes

### Example 1: Exemplary Gigasome Production Methods

### Neuronal Gigasome Production Via Monoculture

In a first example, primary human neurons or neuronal cell lines as shown in Table 1 are established and cultured in multi-well plates. To induce increased gigasome production, the neuronal cultures are treated with one of the compounds as shown in Table 2, or with RNAi targeting genes as shown in Table 3.

TABLE 1
Exemplary neuronal cell models used for gigasome production

| Name | Species | Cell Type | Supplier |
|---|---|---|---|
| HBEC-5i | Human | Cerebral microvascular | ATCC |
| | | endothelium | (CRL-3245) |
| ReNcell CX Human | Human | Neural progenitor | Millipore |
| Neural Progenitor | | | (SCC007) |
| Cell Line | | | |
| HCN2 | Human | Cortical Neuron | ATCC |
| | | | (CRL10742) |
| iCell GlutaNeurons | Human | Glutamatergic-enriched | Fujifilm/Cellular |
| | | cortical neurons | Dynamics |
| | | derived from iPSCs | (01279) |
| iPSC neurons | Human | Human iPS cells | Allstem (iP11N) |
| | | (Inducible NGN2) | |
| HT22 | Mouse | Hippocampal | |
| SH-SY5Y | Human | Neuroblast from | ATCC |
| | | neural tissue | (CRL02266) |

TABLE 2
Exemplary compounds used to induce gigasome production

| Compound | Concentration | Duration | Supplier | Reference |
|---|---|---|---|---|
| Rapamycin | 20 nM-2 μM | Up to | Sigma | Nicolas-Avila |
| | | 48 hrs | | (2020) Cell |
| Isoproterenol | 0.1 μM-10 μM | Up to | Sigma | Nicolas-Avila |
| | | 48 hrs | | (2020) Cell |
| Hydrogen | 1 mM-10 mM | Up to | Sigma | Fu et al. (2019) |
| peroxide | | 48 hrs | | Biorxiv |
| Spautin-1 | 0.1 μM-10 μM | Up to | Sigma | Melentijevic et |
| | | 48 hrs | | al. (2017) |
| | | | | Nature |
| MG-132 | 0.1 μM-10 μM | Up to | Sigma | Melentijevic et |
| | | 48 hrs | | al. (2017) |
| | | | | Nature |
| Chloramphenicol | 0.5 μg/ml- | Up to | Sigma | Tian et al. |
| | 50 μg/ml | 48 hrs | | (2016) |
| | | | | Oncotarget |
| Bafilomycin | 1 nM-1 μM | Up to | Sigma | Redmann et al. |
| | | 48 hrs | | (2017) Redox |
| | | | | Biol. |
| Carbonyl cyanide | 0.1 μM-10 μM | Up to | Sigma | Charan et al. |
| 3-Chlorophenyl- | | 48 hrs | | (2014) Cell |
| hydrazone | | | | Death and |
| (CCCP) | | | | Disease |

TABLE 3
Exemplary gene targets to knock-down or
knock-out to induce gigasome production
Gene
HSF-1
ATG7
BECN1
IGG-1/2
UBL5
PINK1
DCT1
PDR1
MTORC
AKT

### Neuronal Gigasome Production Via Co-Culture

In a second example, primary human neurons or neuronal cell lines are established and co-cultured with primary human microglia cells or microglial cell lines (Table 4). Co-culture is established using three different methods: 1) direct co-culture of the neuronal cells and microglia in the same wells using multi-well plates; 2) separation of neuronal cells and microglia using a transwell insert; or 3) direct co-culture of neuronal cells and microglia in an organoid system. To induce increased gigasome production, the cultures are treated with a compound as shown in Table 5, or with RNAi targeting genes as shown in Table 6.

TABLE 4
List of exemplary neuronal cell models used for gigasome production

| Name | Species | Cell Type | Supplier |
|---|---|---|---|
| HBEC-5i | Human | Cerebral | ATCC |
| | | microvascular | (CRL-3245) |
| | | endothelium | |
| ReNcell CX Human | Human | Neural progenitor | Millipore |
| Neural Progenitor | | | (SCC007) |
| Cell Line | | | |
| HCN2 | Human | Cortical Neuron | ATCC |
| | | | (CRL10742) |
| iCell | Human | Glutamatergic- | Fujifilm/Cellular |
| GlutaNeurons | | enriched cortical | Dynamics |
| | | neurons derived | (R1061) |
| | | from iPSCs | |
| iPSCs | Human | | Patient-derived |
| HT22 | Mouse | Hippocampal | |
| SH-SY5Y | Human | | |
| Microglia | Human | Microglia | Accegen |
| | | | (ABC-TC3704) |
| iCell Microglia | Human | Microglia | Fujifilm/Cellular |
| | | | Dynamics (R1131) |
| iCell Microglia | Human | Microglia | Fujifilm/Cellular |
| AD TREM2 | | | Dynamics (R1202) |
| CHME-5 | Human | Embryonic spinal | |
| | | cord/cortex | |
| | | microglia | |
| HMO6 | Human | Embryonic | |
| | | telencephalon | |
| | | microglia | |
| Huμglia | Human | Adult cortex | |
| | | microglia | |
| HMC3 | Human | Microglia | ATCC |
| | | | (CRL-3304) |

TABLE 5
Exemplary compounds used to induce gigasome production

| Compound | Concentration | Duration | Supplier | Reference |
|---|---|---|---|---|
| Rapamycin | 20 nM-2 μM | Up to 48 hrs | Sigma | Nicolas-Avila |
| | | | | (2020) Cell |
| Isoproterenol | 0.1 μM-10 μM | Up to 48 hrs | Sigma | Nicolas-Avila |
| | | | | (2020) Cell |
| Hydrogen peroxide | 1 mM-10 mM | Up to 48 hrs | Sigma | Fu et al. (2019) |
| | | | | Biorxiv |
| Spautin-1 | 0.1 μM-10 μM | Up to 48 hrs | Sigma | Melentijevic et |
| | | | | al. (2017) |
| | | | | Nature |
| MG-132 | 0.1 μM-10 μM | Up to 48 hrs | Sigma | Melentijevic et |
| | | | | al. (2017) |
| | | | | Nature |
| Chloramphenicol | 0.5 μg/ml-50 μg/ml | Up to 48 hrs | Sigma | Tian et al. |
| | | | | (2016) |
| | | | | Oncotarget |
| Bafilomycin | 1 nM-1 μM | Up to 48 hrs | Sigma | Redmann et al. |
| | | | | (2017) Redox |
| | | | | Biol. |
| Carbonyl cyanide 3- | 0.1 μM-10 μM | Up to 48 hrs | Sigma | Charan et al. |
| Chlorophenylhydrazone | | | | (2014) Cell |
| (CCCP) | | | | Death and |
| | | | | Disease |

TABLE 6
Exemplary gene targets to target via RNAi to induce gigasome production
Gene

HSF-1
ATG7
BECN1
IGG-1/2
UBL5
PINK 1
DCT1
PDR1
MTORC
AKT

### Example 2: Quantification and Characterization of Neuronal Gigasome Production

Production and characterization of gigasome produced, for example, as described by the monoculture and co-culture methods described in Example 1, are assessed using flow cytometry, live microscopy, and/or immunofluorescent microscopy.

### Gigasome Quantification Using Flow Cytometry

To harvest gigasomes, cell culture media is transferred from cell culture plates into 1.5 mL centrifuge tubes. The samples are centrifuged at 50× g for 5 minutes at 4° C. The resulting supernatant is transferred to a new tube, and the pellet of cells is stored for future analysis. The samples are centrifuged at 300× g for 5 minutes at 4° C. The supernatant is transferred to a new tube. The samples are then centrifuged at 1,000× g for 5 minutes at 4° C. The supernatant is discarded and the pellet of enriched gigasomes are resuspended in 100 μL of sorting buffer that contains a detection reagent (e.g., an antibody or stain). The stain may include, e.g., MitoTracker Deep Red to identify the presence of mitochondria, or Lysotracker to identify the presence of lysosomes. The gigasomes and sorting buffer are incubated for 15 minutes at 4° C. for 15 minutes. After incubation, an additional 1-2 mL of sorting buffer is added to wash any excess stain and the sample is centrifuged at 1,000× g for 5 minutes at 4° C. The supernatant is discarded. The pellet is then resuspended with 1 mL of sorting buffer containing a nuclear stain (e.g., Draq5 at a 1:5,000 dilution) to distinguish nucleated versus non-nucleated structures.

Gigasomes are identified and sorted according to the following gating strategy: Logarithmic scale and peak height are used. Event level below 100 events/s with 1.5 μL/minute flow rate and 150 mbar pressure was considered acceptable for background. Three washing cycles were performed between the samples. Flow rate was adjusted between 1.5-4.5 μL/minute to keep average event rates below 3000 events/second. Gigasomes are identified as among particles with the highest FSC-A and SSC-A signal in the 1,000× g pellet obtained above. Doublets using FS-H and FSC-W and particles containing DNA (Draq5+) are discarded. If another fluorescent marker or stain is used, that signal is used to ensure sorting of specific gigasomes (e.g., those that contain mitochondria). Quantification of fluorescence is performed by comparing cell fluorescence with known external standards by using commercially available beads. Statistical analysis is performed using Tukey's multiple comparison test. Gigasomes may also be distinguished by their size by calibrating proper instrument settings using a set of polystyrene microparticles of varying sizes that may serve as references.

### Gigasome Quantification and Characterization Using Live Microscopy

To study gigasome production using live microscopy, live neuronal cell cultures are stained with Hoechst 33342 and CellTracker Green to identify nuclei and cytoplasm, respectively. Other stains can be used to identify various organelles, for example, CellLight Mitochondria or MitoTracker (mitochondria), CellLight-Lysosomes (lysosomes), HCS LipidTOX Neutral Lipid Stain (lipid vesicles), CellLight-ER (endoplasmic reticulum), and/or CellLIght Golgi (Golgi complexes). Gigasome production is then induced as described above in Example 1. Cell cultures are imaged under a time lapse for up to 48 hours and 3D reconstructions with Z stacks of 0.25 μm are taken. Gigasomes are identified and quantified as CellTracker-positive, Hoechst 33342-negative spheres between 1-20 μm in diameter. The presence of specific organelles in neuronal cells and gigasomes are measured via respective fluorescent signals. 3D features of cells and gigasomes can also be reconstructed using software such as Imaris (Bitplane AG).

### Gigasome Quantification and Characterization Using Immunofluorescent Microscopy

After induction of gigasome production, live neuronal cell cultures are washed with phosphate buffered saline (PBS) and fixed using paraformaldehyde, formaldehyde, or 100% methanol as appropriate. The fixed samples are incubated with primary antibodies as shown in Table 7 and are counterstained with the appropriate secondary antibodies and DAPI nuclear stain.

For image analysis: Gigasomes are identified and quantified as distinct circular and spherical vesicles between 1-20 μm in diameter and are DAPI-negative. The presence of specific proteins of interest as shown in Table 7 in the neuronal cells and the gigasomes is quantified as a measure of fluorescent intensity. 3D features of the neuronal cells and gigasomes are further reconstructed using Imaris software.

TABLE 7
Exemplary primary antibodies used to detect proteins of interest

| Primary antibody | Species | Supplier |
|---|---|---|
| Beta amyloid | Rabbit | Abcam |
| Tau | Rabbit | Abcam |

### Example 3: Quantification and Characterization of Neuronal Gigasome Phagocytosis by Microglia

Phagocytosis of gigasomes by microglia in co-culture conditions are assessed in one or more of the following ways: 1) flow cytometry; 2) live microscopy; and/or 3) immunofluorescent microscopy.

### Assessment of Microglial Phagocytosis of Gigasomes Via Flow Cytometry

Live neuronal cell cultures are stained with CellTracker Green or tagged with green fluorescent protein (GFP) and live microglia are stained with CellTracker Red in one of the co-culture conditions as described in Example 1. The microglia are harvested from the cell culture media by trypsinization followed by pipetting media from the cell culture plates into 1.5 mL centrifuge tubes. The sample is centrifuged at 50× g for 5 minutes at 4° C. and the supernatant is transferred to a new tube. The pellet of cells is resuspended in 100 μL sorting buffer containing antibodies to target marker proteins for 15 minutes at 4° C. in the dark. For example, for microglia, an anti-CD11b antibody at a 1:200 dilution and/or an anti-ACSA2 antibody is used. After incubation, an additional 1-2 mL of sorting buffer is added to wash off excess antibody, and the sample is centrifuged at 1,000× g for 5 minutes at 4° C. The supernatant is discarded. The pellet is resuspended with 1 mL sorting buffer containing Draq5 (1:5,000 dilution) to discriminate nucleated versus non-nucleated structures. 1.5 mL tubes are prepared with 100 mL of collection buffer. Microglia are identified and sorted according to the following gating strategy: microglia are identified as having highest CD11b and ACSA2 signal or CellTracker Green signal; particles without DNA (Draq5−) are discarded; sorting for microglia stained with CellTracker Red indicating phagocytosis of neuronal tissue.

Each condition will be scored for phagocytosis and plotted to observe differences in phagocytosis under different conditions. The phagocytic score is calculated as the percentage of cells taking up Green/Red stain×mean fluorescent intensity/1,000). Other descriptive measures (median, interquartile range, frequency, and percent) will also be used to summarize the data. Paired t-tests are used to compare phagocytic activity induced by various compounds in Table 5 and genetic alterations listed in Table 6. Values of P≤0.05 are considered significant. Statistical analysis and graphing are performed using GraphPad Prism Software (La Jolla, CA).

### Assessment of Microglial Phagocytosis of Gigasomes Via Live Microscopy

To study gigasome production using live microscopy, live neuronal cell cultures are stained with Hoechst 33342 and CellTracker Green and live microglia are stained with Hoechst 33342 and CellTracker Red in one of the co-culture conditions as described in Example 1. Other stains can be used to identify various organelles, for example, CellLight Mitochondria or MitoTracker (mitochondria), CellLight-Lysosomes (lysosomes), HCS LipidTOX Neutral Lipid Stain (lipid vesicles), CellLight-ER (endoplasmic reticulum), and/or CellLIght Golgi (Golgi complexes). Gigasome production is then induced as described in Example 1. Cell cultures are imaged under a time lapse for up to 48 hours and 3D reconstructions with Z stacks of 0.25 μm are taken. Gigasomes are identified and quantified as CellTracker or GFP+/Hoechst 33342-spheres between 1-20 μm in diameter. For quantification, parameters are defined to gate the circularity and diameter of the particles. This provides information regarding the area of a gigasome, the mean intensity, and the number of gigasomes surrounding the neurons as well as within the microglia. To visualize uptake within the microglia, cells are assessed by the presence of GFP within the intracellular space; presence of specific organelles in neuronal cells and gigasomes are measured via respective fluorescent signals; and 3D features of neuronal cells and gigasomes are further reconstructed using Imaris software.

### Microglia Quantification and Characterization Using Immunofluorescent Microscopy

After induction of gigasome production, live neuronal cell cultures are washed with PBS and fixed using paraformaldehyde, formaldehyde, or 100% methanol as appropriate. Fixed samples are incubated with primary antibodies of interest as shown in Table 8 and counterstained with appropriate secondary antibodies and DAPI. For image analysis, microglia are identified as CD11b+; phagocytosed gigasomes are identified and quantified as distinct circular/spherical vehicles between 1-20 um in diameter that is CellTracker Green+/Hoechst 33342−; the presence of specific organelles in neuronal cells and gigasomes are measured via respective fluorescent signals; and 3D features of neuronal cells and gigasomes are further reconstructed using Imaris software (Bitplane AG).

TABLE 8
Exemplary primary antibodies used to
stain specific proteins of interest

| Primary antibody | Species | Supplier |
|---|---|---|
| Beta amyloid | Rabbit | Abcam |
| Tau | Rabbit | Abcam |
| CD11b | Rabbit | Abcam |

### Western Blotting

Microglial cells co-cultured with neurons are lysed using RIPA buffer for the presence of GFP protein and fluorescent signal from tagged organelles. The media may also be used to characterize gigasomes by Western blotting if enriched in the media. Protein concentration is measured by Bicinchonic acid assay kit to calculate the total protein in each sample. Samples are prepared with 4×Laemmli sample buffer with 10% β-mercaptoethanol. Samples are resolved on 4-20% gradient SDS-PAGE. Proteins are transferred to PVDF membrane. GFP protein is probed with an anti-GFP primary antibody overnight at 4° C. The membrane is then incubated with IRDye-conjugated secondary antibody, and signal is detected and imaged using Odyssey CLx imaging system.

### Mass Spectrometry

Total protein obtained from the microglial cell cultures are used for high performance liquid chromatography in tandem with mass spectrometry (LC-MS). Progenesis QI for proteomics software is used for processing of raw files. Peptide identification is run with Uniprot human FASTA sequences. Label-free protein quantification is performed with Hi-N method. Data is analyzed using Progenesis QI for Proteomics with trypsin using cysteine carbamidomethylation as a fixed modification, methionine oxidation and protein N-terminal acetylation as variable modifications, allowing for up to 2 missed cleavage sites, precursor ion mass tolerance at 4.5 ppm and fragment ion mass tolerance at 20 ppm, and false discovery rate at 1 percent for both peptide spectrum match and protein identifications.

After extraction of data, non-specific proteins were discarded based on information from a database of common MS contaminants (www.crapome.org) using a protein occurrence cut-off of more than 10% across all mass spectrometry data for both peptide spectrum match and protein sets present in the database. The peptide error tolerance is set to a maximum of 10 ppm and the false discovery rate limited to less than 1% and default values in the software are used for the rest of the parameters. Presence of GFP protein peptides and fluorescent peptides within microglia are indicative of neuronal material within the microglial cells. Functional annotation and enrichment analysis of the proteins can also be conducted to identify unique gigasome markers using the Kyoto Encyclopedia of Genes and Genomes (KEGG) and the Gene Ontology (GO) databases.

### Example 4: In Vivo Exopher Induction in Mice

Mice are injected three times a week with rapamycin (4 m/kg body weight) intraperitoneally for 2 weeks to induce exopher production in vivo. At 2 weeks, mice are anesthetized, and the following tissues are harvested: brain, heart, lung, liver, stomach, spleen, kidneys, intestine, muscle, and skin. All tissues are separately minced into small pieces, digested in Hank's balanced salt solution (HBSS) with liberase (1 U/mL) and DNase I (10 mU/mL) for 40 minutes at 37° C. The samples are mixed with gentle pipetting to obtain single cell suspensions. Samples are centrifuged at 50× g for 5 minutes at 4° C. and the supernatant is transferred into a new tube. The pellet of mostly whole cells is saved for later analysis. The sample is centrifuged at 300× g for 5 minutes at 4° C., the supernatant is discarded, and the exopher-enriched pellet is kept for determining which tissues contain or produce exophers.

The pellet is resuspended in 100 μL of sorting buffer that contains antibodies at desired concentrations and incubate for 15 minutes at 4° C. in the dark. For example, for cardiac exophers, an anti-CD31 antibody is used for exclusion of endothelial cell-derived particles to clean the gating strategy. Also, for example, exophers can be stained with MitoTracker Deep Red to identify the presence of mitochondria. Sorting buffer is added (1-2 mL) to the sample to wash off excess antibody or stain, and the sample is centrifuged at 1,000× g for 5 minutes at 4° C. The supernatant is discarded and the pellet is resuspended with 1 mL of sorting buffer containing Draq5 to discriminate between nucleated and non-nucleated structures.

Exophers are identified and sorted according to the following gating strategy in an Attune N×T Flow Cytometer and data are analyzed using FlowJo software: exophers are identified as among particles with the highest FSC-A and SSC-A signal in the 1,000× g pellet obtained above. Doublets using FS-H and FSC-W and particles containing DNA (Draq5−) or endothelial markers (e.g., CD31+) are discarded. If another fluorescent marker or stain is used, that signal is used to ensure sorting of specific exophers (e.g., those that contain mitochondria).

The results of these assays will demonstrate which of the tissues tested contain or do not contain exophers.

### Example 5: Assessment of Gigasome Production by Confocal Microscopy

Gigasomes produced as described herein (e.g., using neuronal cell lines, such as SH-SY5Y cells, co-cultured with human microglial clone 3 cell line HMC3 cells) can be assessed by confocal microscopy. In this example, neurons are tagged with GFP to differentiate from the microglia. The cells are incubated with various stressors for efficient induction of gigasomes for maximal visualization. The neurons and microglia can be co-cultured directly in the same wells, or co-cultured separately (e.g., in culture inserts (Ibidi), and allowed to mix after removal of the insert), or cultured transwell insert (pore size-8 μm), and then transferred onto a plate containing cultured microglial cells.

Nuclei will be stained with Hoescht 33342 and other organelles will be stained as listed below:
1. In some experiments, cells are stained with Mitoview 640 (Biotium) to identify mitochondria.
2. In some experiments, cells are stained with CellLight-Lysosomes (Thermo) to identify lysosomes.
3. In some experiments, cells are stained with HCS LipidTOX Neutral Lipid Stain (Thermo) for lipid vesicles.
4. In some experiments, cells are stained with CellLight-ER (Thermo) to identify endoplasmic reticulum.
5. In some experiments, cells are stained with CellLight Golgi (Thermo) to identify Golgi complexes.

Cell cultures are imaged under time lapse for up to 24 hours using a confocal microscope. For 3D reconstructions, Z stacks of 0.25 um are taken. For image analysis, ImageJ and Zeiss Zen Blue software will be used. Gigasomes are identified and quantified as distinct circular/spherical vehicles in the extracellular space of the neurons, between 1-20 um in diameter. Gigasomes will generally be Hoechst 33342-negative but GFP-positive. For quantification, parameters are defined to gate the circularity and diameter of the particles, thereby permitting determination of the area of the gigasome, the mean intensity, and the number of gigasomes surrounding the neurons as well as within the microglia. The presence of specific organelles in neuronal cells and gigasomes are measured via respective fluorescent signals.

To visualize uptake within the microglia, cells will be assessed for the presence of GFP with the intracellular space, as well as the presence of the respective fluorescent signals emitting from the stained organelles. These would be quantified as described above.

### Example 6: Identification and Isolation of Gigasomes by Flow Cytometry

In this example, harvested gigasomes are identified and sorted by flow cytometry. Briefly, gigasomes are harvested from the cell culture media by pipetting media from cell culture plates into 1.5 mL Eppendorf tubes. Samples can be centrifuged at 50 g for 5 minutes at 4° C. and supernatant can be transferred into new tubes. The pellet can be saved for later analysis. The samples are then centrifuged at 300 g for 5 minutes at 4° C. and supernatant is transferred into a new tube. The pellet, which contains mostly whole cells, is saved for later analysis. Samples are then centrifuged at 1,000 g for 5 minutes at 4° C. The supernatant is discarded, and the pellet (which is enriched in gigasomes) is kept. The pellet is then resuspended in 100 uL of sorting buffer (e.g., which contains antibodies at desired concentrations) and incubated for 15 minutes at 4° C. in the dark. For gigasomes produced by cardiac cells, anti-CD31 antibodies (1:200 dilution) can be used for exclusion of endothelial cell derived particles to clean gating strategy (e.g., as described in Nicolas-Avila et al. 2020 and Pinto et al. 2016; incorporated herein by reference in their entirety). In some instances, gigasomes are stained with Mitotracker Deep Red (Thermo) to identify presence of mitochondria. 1-2 mL of sorting buffer can be added to wash off excess antibody. The sample can then be centrifuged at 1000 g for another 5 minutes at 4° C., and the pellet resuspended in 1 mL sorting buffer containing Draq5 (1:5000 dilution), a DNA probe that allows discrimination of nucleated versus non-nucleated structures. Keep samples at 4 degrees in the dark.

Gigasomes are identified and sorted into 1.5 mL Falcon tubes (each containing 100 mL of collection buffer) according to the following gating strategy in a cytometer equipped with specific lasers and specific filters is used for identifying, characterizing and quantifying gigasomes. The data are analyzed using FlowJo software. Logarithmic scale and peak height are used. Event level below 100 events/s with 1.5 μL/minute flow rate and 150 mbar pressure are considered acceptable for background.

Three washing cycles are performed between the samples. Flow rate is adjusted between 1.5-4.5 L/minute to keep average event rates below 3000 events/second. Gigasomes are among particles with highest FSC-A and SSC-A signal in the 1,000 g pellet obtained above. Doublets are discarded using FSC-H and FSC-W. Particles containing DNA (Draq5+) or endothelial markers (CD31+) can also be discarded. If the samples were stained with another fluorescent signal, that signal can be used to sort specific gigasomes of interest (e.g., gigasomes containing mitochondria). Fluorescence can be quantified, for example, by comparing cell fluorescence with known external standards, e.g., using commercially available beads. Statistical analysis is conducted using Tukey's multiple comparison test. Gigasomes can also be distinguished by their size, for example, by calibrating proper instrument settings using a set of polystyrene microparticles of varying sizes (1-20 μm in sizes) that will serve as references. Gigasomes can be quantified using the methods and parameters described above.

### Example 7: Biochemistry for Detecting Transfer of Proteins Delivered by Gigasomes

In this example, microglial cells co-cultured with neurons are tested for the presence of proteins transferred from the neurons via gigasomes. In brief, microglial cells co-cultured with neurons in transwell inserts will be lysed using RIPA buffer for the presence of GFP protein and fluorescent signal from tagged organelles. If gigasomes are enriched enough in the media to be detected by western blotting, media will also be used to biochemically characterize gigasomes.

Protein concentration is measured using the Bicinchonic acid assay kit (Thermo Fisher Scientific) to determine total protein concentration. Samples are prepared with 4× Laemmli sample buffer with 10% β-mercaptoethanol and run on 4-20% gradient SDS-PAGE (Mini-Protean TGX Precast protein gel, Bio-Rad, Hercules, USA). For Western blot analysis, proteins are transferred to PVDF membrane (Immobilon-P, Merck Millipore, Burlington, USA) at 200 mA for 90 minutes using wet transfer. Nonspecific binding is blocked by with 3% BSA in Tris-buffered saline containing 0.1% Tween-20 (TBST) for 1 hour followed by primary antibody incubation anti-GFP overnight at 4° C. The membranes are washed three times with TBST followed by incubation with IRDye-conjugated secondary antibody in dilution 1:15,000 (Licor) for 1 hr. After washing, the signal is detected and imaged using Odyssey CLx imaging system (Licor).

In some instances, blots are assessed for the presence of GFP-positive signal within the microglia. Detection of GFP in microglial cells indicates successful delivery from the neurons in the co-culture via gigasomes.

### Example 8: Mass Spectrometry

In this example, total protein obtained from the microglial cell cultures described in Example 7 will be used for high performance liquid chromatography in tandem with mass spectrometry (LC-MS). This will allow further confirmation of delivery of payload proteins to the recipient microglial cells, as well as characterization of the proteomes of the recipient cells. In brief, peptide identification will be run with Uniprot human FASTA sequences and label-free protein quantification will be performed with Hi-N method (Protein Lynx Global Server) (e.g., as described in Silva et al, 2006; incorporated herein by reference in its entirety). Data is analyzed using Progenesis QI with trypsin as a digesting reagent, using cysteine carbamidomethylation as a fixed modification, methionine oxidation and protein N-terminal acetylation as variable modifications, allowing for up to 2 missed cleavage sites, precursor ion mass tolerance at 4.5 ppm and fragment ion mass tolerance at 20 ppm, and false discovery rate at 1 percent for both peptide spectrum match and protein identifications. After extraction of data, non-specific proteins can be discarded based on information from a database of common MS contaminants (www.crapome.org) using a protein occurrence cut-off of more than 10% across all mass spectrometry data for both peptide spectrum match and protein sets present in the database. The peptide error tolerance is set to a maximum of 10 ppm and the false discovery rate limited to less than 1% and default values in the software were used for the rest of the parameters.

The presence of GFP protein peptides and fluorescent peptides within microglia would be indicative of neuronal material within the microglial cells. Functional annotation and enrichment analysis of the proteins can also be conducted to identify unique gigasome markers using the Kyoto Encyclopedia of Genes and Genomes (KEGG) and the Gene Ontology (GO) databases.

### Example 9: Characterization of Producer Cells in the Process of Making Gigasomes

To characterize the producer cells in the process of making gigasomes using live microscopy, live neuronal cell cultures are stained with Hoechst 33342 and CellTracker Green to identify nuclei and cytoplasm, respectively. In some cases, live neuronal cell cultures are also stained with Image-iT LIVE Red Caspase-3 and -7 Detection Kit (ThermoFisher Scientific) to assess for producer cell viability. In some experiments, producer cells are stained with Mitoview 640 (Biotium) to identify mitochondria. In some experiments, producer cells are stained with CellLight-Lysosomes (Thermo) to identify lysosomes. In some experiments, producer cells are stained with HCS LipidTOX Neutral Lipid Stain (Thermo) for lipid vesicles. In some experiments, producer cells are stained with CellLight-ER (Thermo) to identify endoplasmic reticulum. In some experiments, producer cells are stained with CellLight Golgi (Thermo) to identify Golgi complexes.

Live neuronal cells are induced to produce gigasomes as described above in Example 1. Successful producer cells of interest at identified as having produced a gigasome within 48 hours using live microscopy. Temporal changes to the producer cell viability and/or quantity of mitochondria, lysosomes, lipid vesicles, endoplasmic reticulum, and/or Golgi complexes within the producer cell will be assessed before and after gigasome production by the producer cell.

### Example 10: Exemplary Gigasome Production Methods Using Compounds

### Neuronal Gigasome Production Via Monoculture

This example shows that a neuronal cell line and a cardiomyocyte cell line can be induced to produce gigasomes using various compounds with distinct mechanisms of action. In a first demonstration, a neuronal cell line derived from human neuroblastoma was used to induce gigasome production. Some of the experiments were performed in these neuronal cells stably overexpressing GFP protein. Cells were plated at various densities in multi-well glass-bottom plates to observe the production of gigasomes. To further induce gigasome production, the neuronal cultures were treated with compounds either alone or in combination as shown in Table 9.

TABLE 9
Exemplary compounds used to induce gigasome production.

| Compound | Concentration | Duration | Mechanism of action |
|---|---|---|---|
| Rapamycin | 20 nM-2 μM | Up to 48 hrs | Autophagy inducer |
| MG-132 | 1 nM-10 μM | Up to 48 hrs | Proteasomal inhibitor |
| Spautin-1 | 0.05 μM-5 μM | Up to 48 hrs | Inhibitor of autophagy |
| Bafilomycin-A1 | 0.1 nM-100 nM | Up to 48 hrs | Inhibitor of autophagosome-lysosome fusion |

### Cardiomyocyte Gigasome Production Via Monoculture

To observe gigasome production, a cardiomyocyte cell line derived from human ventricular heart tissue was used to induce gigasome production. Cells were plated at various densities in multi-well glass-bottom plates to observe the production of gigasomes. To induce gigasome production, the cardiomyocyte cell cultures were treated with the compounds either alone or in combination as shown in Table 10.

TABLE 10
Exemplary compounds used to induce gigasome production in cardiomyocytes

| Compound | Concentration | Duration | Mechanism of action |
|---|---|---|---|
| Rapamycin | 20 nM-2μM | Up to 48 hrs | Autophagy inducer |
| AZD2014 (Vistusertib) | 10-100 nM | Up to 48 hrs | Autophagy inducer |
| MG-132 | 1 nM-10 μM | Up to 48 hrs | Proteasomal inhibitor |
| Spautin-1 | 0.05 μM-5 μM | Up to 48 hrs | Inhibitor of autophagy |
| Bafilomycin-A1 | 0.1 nM-100 nM | Up to 48 hrs | Inhibitor of autophagosome-lysosome fusion |

### Example 11: Visualization of Gigasome Generation Process and Characterization of Gigasome Cargo

This example shows that gigasomes can be visualized being produced from a parent cell of neuronal or cardiomyocyte origin and that they contain cellular organelles but not nuclear material. Visualization and characterization of gigasomes produced, for example, as described by the monoculture and co-culture methods described in Example 1, were assessed using live microscopy and immunofluorescent microscopy.

### Visualization of Neuronal Gigasome Generation Process Using Live Microscopy

To study gigasome production using live microscopy, live neuronal cell cultures were plated in 24 well plates at a density ranging from 50K-125K cells per well. Cells were stained with Hoechst 33342 to visualize nuclei and nuclear material, CellTracker Green to visualize cytoplasm, and MitoTracker Deep Red to visualize mitochondria. Gigasome production was then induced with various drugs as described in Example 1. Cell cultures were imaged every 15-30 minutes under a time lapse for up to 48 hours (FIGS. 1A and 1B). For image analysis, ImageJ and Zeiss ZEN Blue software was used. Gigasomes were identified and quantified as distinct circular/spherical vesicles in the extracellular space of the neurons, between 1-20 μm in diameter. Gigasomes were identified based on having positive neuronal cytoplasmic signal and negative Hoechst 33342 signal. The presence of mitochondria in gigasomes was measured via respective fluorescent signal.

Time-lapse montages were produced and edited to show the formation of gigasomes from parent neuronal cells after treatment with 0.1 M MG-132 (FIGS. 1A and 1B). Gigasomes and parent cells were identified and tracked throughout the duration of the time lapse. In some experiments, gigasomes produced by parent cells in MG-132 treatment groups were identified. An increase in gigasome production was observed in the MG-132 treated cells as compared to the DMSO control condition.

### Characterization of Organelle Cargo of Neuronal Gigasome Using Immunofluorescent Microscopy

After induction of gigasome production, live neuronal cell cultures were washed with phosphate buffered saline (PBS) and fixed using 4% paraformaldehyde or 100% methanol as appropriate. The fixed samples were blocked with the appropriate blocking buffer, incubated with primary antibody against beta III tubulin for cytosolic regions (Cell Signaling; Mouse), incubated with primary antibody against TOM20 (Cell Signaling; Rabbit) for mitochondria or LAMP1 (Cell Signaling; Rabbit) for lysosomes, and then counterstained with the appropriate anti-rabbit or anti-mouse secondary antibodies, and DAPI nuclear stain.

Gigasomes were identified as distinct circular and spherical and DAPI-negative vesicles between 1-20 μm in diameter. The presence of specific proteins of interest in the neuronal cells and the gigasomes was determined based on the fluorescent intensity of specific markers.

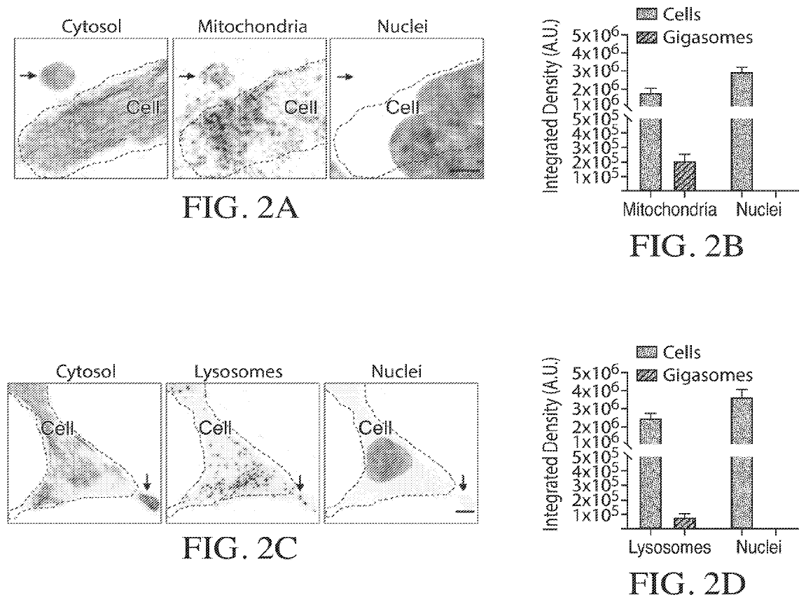

Induction of gigasome production with various compounds in neuronal cells effectively increased the number of gigasomes characterized by an average diameter size of 5-10 μm, absence of nuclear marker stain and presence of cytosolic signal (FIGS. 2A and 2C). Furthermore, neuronal gigasomes contained different organelles, including mitochondria (FIG. 2A) and lysosomes (FIG. 2C). To quantify the specific organelle content within the parent cells and gigasomes, a region was interest (ROI) (either the gigasome or the cell) was determined and the sum of the values of all the pixels in the selected object was calculated, termed as integrated density (FIGS. 2B and 2D). Consistent with results from the live imaging described above, gigasomes produced by the cell did not contain any nuclear material (FIGS. 2B and 2D).

### Characterization of Organelle Cargo of Cardiomyocyte Gigasome Using Immunofluorescent Microscopy

After induction of gigasome production, live cardiomyocyte cultures were washed with phosphate buffered saline (PBS) and fixed using 4% paraformaldehyde or 100% methanol as appropriate. The fixed samples were blocked with the appropriate blocking buffer, incubated in CellTracker Green (Thermo) for cytosol, incubated with MitoTracker Deep Red (Thermo) for mitochondria or primary antibody against LAMP1 for lysosomes (Cell Signaling; Rabbit), and then counterstained with the appropriate anti-rabbit secondary antibodies, and Draq5 nuclear stain.

Gigasomes were identified as distinct circular and spherical and DAPI-negative vesicles between 1-20 μm in diameter. The presence of specific proteins of interest in the cardiac cells and the gigasomes was determined based on fluorescent intensities of specific markers.

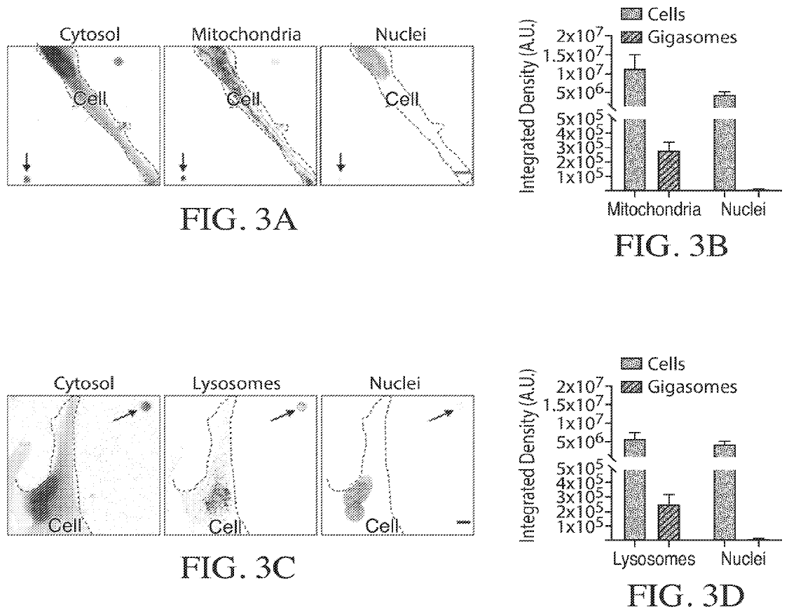

Induction of gigasome production with various compounds in cardiac cells effectively increased the number of gigasomes characterized by an average diameter size of 5-10 m, absence of nuclear marker stain and presence of cytosolic signal (FIGS. 3A and 3C). To quantify the specific organelle content within the parent cells and gigasomes, a region was interest (ROI) (either the gigasome or the cell) was determined and the sum of the values of all the pixels in the selected object was calculated, termed as integrated density (FIGS. 3B and 3D). Consistent with results from the live imaging described above, gigasomes produced by the cell did not contain any nuclear material (FIGS. 3B and 3D).

### Example 12: Modulation and Quantification of Gigasome Yield, Purity, and Distribution

### Modulation and Quantification of Neuronal Gigasomes

This example shows that gigasomes can be differentially induced upon treatment with various compounds alone or in combination as stated in Table 9 and Table 10.

To quantify and characterize gigasomes using microscopy, gigasomes were harvested from cell culture media supernatants. In some experiments, neuronal cells were cultured. In some experiments, cardiomyocytes were cultured. Cells were incubated with Hoechst 33342, CellTracker Green and MitoTracker Deep Red stains to detect nuclear material, cytoplasm and mitochondria, respectively, and cultured in 24-well glass bottom plates at 42,000 cells/cm² and allowed to adhere overnight. Gigasome production was then induced by addition of various compounds as described in Examples 1 and 10. In some experiments, compounds were added either alone or in combination, and a vehicular control amount of DMSO were added to the existing cell culture media to comparatively study gigasome production rates. Cultures were incubated for 24 hours under gigasome inducing conditions.

Cell culture media supernatant samples were centrifuged at 50× g for 5 minutes at 4° C. The supernatant was then transferred into a new tube and centrifuged at 300× g for 5 minutes at 4° C. The resulting supernatants were then centrifuged at 1,000× g for 5 minutes at 4° C., resulting in an enriched pellet of gigasomes. The resulting pellet of cells and cellular debris after the 50× g and 300× g spins were saved for down-stream analysis. The enriched gigasome pellets in the 1000× g spins were washed with buffer (PBS) before being resuspended in 100 μL of PBS and plated in a 96-well plate for confocal microscopy.

The Zeiss ZEN Blue software was used to distribute imaging fields of view (positions) to create an unbiased imaging sample of a well. Images were taken using a Zeiss LSM 900 confocal microscope. Images were in 16-bit format, and contained at least an ESID (brightfield) channel, a Hoechst 33342 fluorescence channel, and a MitoTracker Deep Red fluorescence channel. In some experiments, images also contained a CellTracker Green fluorescence channel. In some experiments, 15 images per well were taken. In some experiments, 10 images per well were taken for cellular pellet samples, and 30 images per well were taken for gigasome enriched pellet samples. ImageJ was used to impose a threshold to create a mask of objects in the field of view. In some experiments, the variance of the brightfield channel was calculated with a radius of 5 pixels and the threshold positive window was between 5 and 65535 for 16-bit images. In some experiments, the threshold for the CellTracker Green fluorescence channel was set between 5403 and 65535 for 16-bit images. A watershed binary segmentation step was performed to separate grouped objects. For each particle in the mask, characteristic parameters of the particle were calculated and saved. In some experiments, these parameters were the particles area, circularity, mean fluorescence signal in the Hoechst 33342 channel (nuclear signal), and mean fluorescence signal in the MitoTracker Deep Red channel (mitochondrial signal).

Particles of interest were identified by thresholding by area and circularity. In some experiments, area was restricted to be between 0.78 and 314 μm² (which correspond to spheres with diameters ranging from 1 to 20 μm). Gigasomes were identified from the particles of interest by thresholding by mean nuclear signal. In some experiments, nuclear signal threshold was set using a value equal to half of a threshold value calculated by the Li auto-threshold algorithm applied to the distribution of all particles of interest. Particles of interest with nuclear signal below this value were considered “negative” for nuclear material and were identified as gigasomes. In some experiments, gigasomes were further analyzed by mitochondrial signal. Mitochondria-positive gigasomes were identified from the particles of interest by thresholding by mitochondrial signal. In some experiments, gigasomes with a mitochondrial mean value between 3050 and 65535 were considered mitochondria-positive gigasomes.

In some experiments, a sampling factor was calculated to extrapolate the total quantity of a type of object (particles, particles of interest, gigasomes, or mitochondrial gigasomes) in the well. The sampling factor was the percentage of the well's area that was imaged. Dividing a quantity or distribution of objects by the sampling factor resulted in an estimated quantity or distribution of objects for the whole well. In some experiments, the number of cells in the cell cultures were estimated with a cell counter. Dividing a quantity or distribution of objects by the number of cells from the culture that produced them resulted in a normalized quantity or distribution. In some experiments, the normalized quantity or distribution of objects was described as a quantity or distribution of objects per 1000 cells.

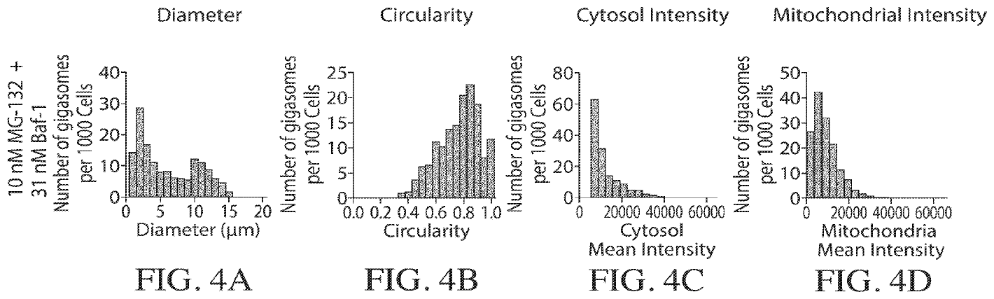

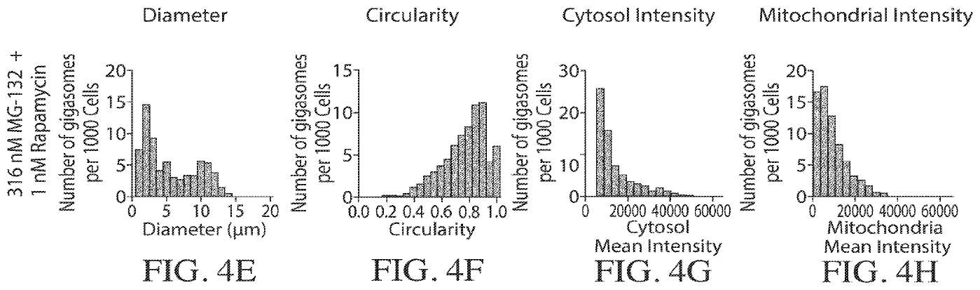

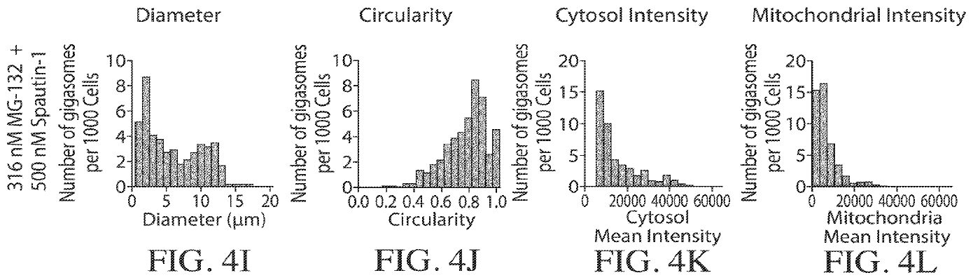

To characterize the gigasomes produced by the various compounds, different parameters of the enriched gigasomes were quantified and plotted (FIG. 4A-4L). In this example, the characteristics of the gigasomes produced by neuronal cells treated with 10 nM MG-132 in combination with 31 nM BafA1 (FIG. 4A-4D), 316 nM MG-132 in combination with 1 nM rapamycin (FIG. 4E-4H) and 316 nM MG-132 in combination with 500 nM Spautin-1 (FIG. 4I-4L) were analyzed. The size analytics of the gigasomes produced showed a similar distribution irrespective of the compound treatment (FIG. 4A, 4E, 4I), even though the number of gigasomes produced differed between treatments. Similarly, the distribution for the circularity of the gigasomes (FIG. 4B, 4F, 4J), the cytoplasmic content (FIG. 4C, 4G, 4K) and the mitochondrial intensity (FIG. 4D, 4H, 4L) were similar across treatments.

Neuronal cells under different drug conditions either alone or in combination displayed differential rates of gigasome production (Table 11). For example, a combination of cells incubated with 316 nM MG-132 and 10 nM rapamycin produced between 50 and 100 gigasomes per 1000 cells, whereas cells incubated with 3 nM MG-132 and 5 M Spautin-1 produced fewer than 10 gigasomes per 1000 cells. Few conditions such as the MG-132 at 10 nM and BafA1 at 31 nM produced higher than 100 gigasomes per 1000 cells (Table 11). Analysis of the percentages of a nuclear material produced by the cells revealed enrichment of the gigasomes in some conditions (between 50-75%) using the differential centrifugation protocol as stated in Example 2. The viability of the parent cells 24 hours after the various drug treatments was also assessed using Cell Titer Glo assay and the MTT assay, and viability is shown in Table 11 as a percentage of viable cells as compared to the control DMSO condition.

TABLE 11
Quantification and characterization of gigasomes enriched from
cell culture media from cells treated with various compounds.

| | | Particles | |
|---|---|---|---|
| | No. of | without | |
| Compound name | gigasomes per | nuclear | |
| (Concentration in nM) | 1000 cells | material (%) | % Viability |
| DMSO | <10 | <50 | 100 |
| MG-132 (3) + Rapamycin (0.1) | <10 | <50 | <50 |
| MG-132 (3) + Rapamycin (1) | <10 | <50 | <50 |
| MG-132 (3) + Rapamycin (10) | <10 | <50 | <50 |
| MG-132 (10) + Rapamycin (0.1) | <10 | <50 | <50 |
| MG-132 (10) + Rapamycin (1) | 10-50 | <50 | <50 |
| MG-132 (10) + Rapamycin (10) | 10-50 | <50 | <50 |
| MG-132 (31) + Rapamycin (0.1) | 10-50 | <50 | <50 |
| MG-132 (31) + Rapamycin (1) | 50-100 | <50 | <50 |
| MG-132 (31) + Rapamycin (10) | 50-100 | <50 | <50 |
| MG-132 (100) + Rapamycin (0.1) | 10-50 | <50 | <50 |
| MG-132 (100) + Rapamycin (1) | 10-50 | <50 | <50 |
| MG-132 (100) + Rapamycin (10) | <10 | <50 | 50-75 |
| MG-132 (316) + Rapamycin (0.1) | 10-50 | <50 | <50 |
| MG-132 (316) + Rapamycin (1) | 50-100 | <50 | 50-75 |
| MG-132 (316) + Rapamycin (10) | 50-100 | <50 | 50-75 |
| MG-132 (3) + Spautin (50) | <10 | <50 | <50 |
| MG-132 (3) + Spautin (500) | <10 | 50-75 | <50 |
| MG-132 (3) + Spautin (5000) | <10 | <50 | 50-75 |
| MG-132 (10) + Spautin (50) | <10 | <50 | 50-75 |
| MG-132 (10) + Spautin (500) | 10-50 | 50-75 | <50 |
| MG-132 (10) + Spautin (5000) | 10-50 | 50-75 | <50 |
| MG-132 (31) + Spautin (50) | 10-50 | 50-75 | <50 |
| MG-132 (31) + Spautin (500) | 10-50 | <50 | <50 |
| MG-132 (31) + Spautin (5000) | 10-50 | 50-75 | <50 |
| MG-132 (100) + Spautin (50) | <10 | <50 | <50 |
| MG-132 (100) + Spautin (500) | 10-50 | <50 | <50 |
| MG-132 (100) + Spautin (5000) | 10-50 | <50 | <50 |
| MG-132 (316) + Spautin (50) | 10-50 | <50 | 50-75 |
| MG-132 (316) + Spautin (500) | 10-50 | <50 | 50-75 |
| MG-132 (316) + Spautin (5000) | 10-50 | <50 | 50-75 |
| MG-132 (3) + BafilomycinA1 (0.1) | <10 | 50-75 | 50-75 |
| MG-132 (3) + BafilomycinA1 (1) | <10 | 50-75 | >75 |
| MG-132 (3) + BafilomycinA1 (10) | 10-50 | <50 | 50-75 |
| MG-132 (3) + BafilomycinA1 (31) | 50-100 | 50-75 | 50-75 |
| MG-132 (3) + BafilomycinA1 (100) | 10-50 | 50-75 | <50 |
| MG-132 (10) + BafilomycinA1 (0.1) | <10 | 50-75 | >75 |
| MG-132 (10) + BafilomycinA1 (1) | <10 | <50 | >75 |
| MG-132 (10) + BafilomycinA1 (10) | <10 | 50-75 | 50-75 |
| MG-132 (10) + BafilomycinA1 (31) | >100 | 50-75 | >75 |
| MG-132 (10) + BafilomycinA1 (100) | 10-50 | 50-75 | <50 |
| MG-132 (31) + BafilomycinA1 (0.1) | 50-100 | <50 | >75 |
| MG-132 (31) + BafilomycinA1 (1) | 50-100 | <50 | >75 |
| MG-132 (31) + BafilomycinA1 (10) | >100 | 50-75 | >75 |
| MG-132 (31) + BafilomycinA1 (31) | >100 | <50 | >75 |
| MG-132 (31) + BafilomycinA1 (100) | 50-100 | 50-75 | <50 |
| MG-132 (100) + BafilomycinA1 (0.1) | 10-50 | <50 | >75 |
| MG-132 (100) + BafilomycinA1 (1) | <10 | 50-75 | >75 |
| MG-132 (100) + BafilomycinA1 (10) | 10-50 | 50-75 | >75 |
| MG-132 (100) + BafilomycinA1 (31) | 50-100 | 50-75 | 50-75 |
| MG-132 (100) + BafilomycinA1 (100) | 10-50 | <50 | <50 |
| MG-132 (316) + BafilomycinA1 (0.1) | 10-50 | <50 | >75 |
| MG-132 (316) + BafilomycinA1 (1) | >100 | <50 | >75 |
| MG-132 (316) + BafilomycinA1 (10) | >100 | <50 | 50-75 |
| MG-132 (316) + BafilomycinA1 (31) | >100 | <50 | 50-75 |
| MG-132 (316) + BafilomycinA1 (100) | 50-100 | 50-75 | <50 |

### Example 13: Gigasome-Mediated Extraction of Disease-Relevant Waste Cargo in Three Neuronal Disease Models In Vitro

This example demonstrates the ability of gigasomes to extract disease-relevant waste cargo in various neuronal disease models in vitro. In some experiments, intracellular accumulation of disease relevant cargo was induced pharmacologically (e.g., by using the γ-secretase inhibitor L-685,458, to trigger intracellular buildup of Alzheimer's disease (AD)-related amyloid precursor protein C-terminal fragments (APP-CTFs) and inhibit Amyloid β peptide formation.) In some experiments, a chemical compound (e.g., sodium arsenite) was used to induce the nuclear-to-cytosolic translocation of an RNA-binding protein, such as HuR, underlying stress granule (SG) formation, membraneless cytosolic compartments containing mRNA-protein complexes. In some experiments, fibrillar protein aggregates were exogenously added to neuronal cell culture media (e.g., fibrils from human P301S mutant tau protein (Tau-F), which play a role in tauopathies and early-onset frontotemporal dementia (FTD).) Analyses of gigasome production in these neuronal disease models were carried out directly on plated cells (either fixed with paraformaldehyde or live) and only in the case of the Tau-F model, gigasomes were also harvested from culture media supernatants. Furthermore, this example demonstrates an increased presence of gigasome-mediated waste cargo extraction when intracellular proteosome functions are inhibited in some disease conditions (e.g., AD-related APP-CTF model and Tau-F model), but not all disease conditions (e.g., stress granule formation did not exhibit further increase).

a. Gigasome-Mediated Extraction of AD-Related APP-CTFs Upon Dual γ-Secretase and Proteasome Inhibition

In this example, neuronal cells were cultured in poly-L-lysine-coated 24-well glass bottom plates at 42,000 cells/cm² and allowed to adhere overnight. Cells were treated with the γ-secretase inhibitor L-685,458 at 5 μM, either alone or in combination with the proteasome inhibitor MG-132 at 0.1 μM. A vehicular control amount of DMSO was added to the media to comparatively study cargo signals within cells and gigasomes. Treatments were carried out in reduced serum-containing media (3% FBS) for 24 hours. Disease-related cargo presence within cells and in gigasomes was characterized and quantified by confocal microscopy analysis of 4% paraformaldehyde fixed cells, following (immuno)fluorescent labeling of cytosolic, nuclear and cargo-specific protein markers. In some experiments β-III tubulin and Alexa Fluor® 488 phalloidin were used to mark the cytosol and proximal plasma membrane, respectively. To detect APP-CTFs, an antibody against the C-terminus of APP (C-APP) was used (see Table 12). Non-specific labeling was blocked by incubation with 5% normal goat serum in PBS in the presence of 0.05% saponin, for 1 h at room temperature. Primary antibodies were incubated overnight at 4° C., followed by incubation with Alexa Fluor®-conjugated secondary antibodies for 1 hour at room temperature. Nuclei were counterstained with DAPI.

TABLE 12
Exemplary reagents and antibodies used for the studies in Example 13

| Reagent | Type | Species | Supplier |
|---|---|---|---|
| L-685,458 | γ-secretase inhibitor | N/A | Tocris Bioscience |
| C-APP | Primary antibody | Rabbit | Sigma |
| Sodium Arsenite | Inorganic compound | N/A | Thermo Fisher Scientific |
| HUR | Primary antibody | Mouse | Abcam |
| Tau441 (2N4R) | Mutant Protein Pre-formed | Human | Eagle Biosciences |
| P301S ATTO 488 | Fibrils (recombinant) | | |
| Alexa Fluor 488 ® | High-affinity filamentous | N/A | Thermo Fisher Scientific |
| Phalloidin | actin (F-actin) probe | | |

Images were taken using a Zeiss LSM 900 confocal microscope using a 40× objective. Images were in 16-bit format and contained fluorescence channels for the cytosolic and F-actin stain (far-red and green, respectively), a DAPI channel for the nucleus and a red channel for the cargo. A range of 5-8 images per well were taken from replicate experiments to capture a final total range of 150-200 cells. ImageJ was used for image analysis as follows. Composite images were separated in the different channels. The cellular cargo fluorescence intensity in the red channel was quantified by subtracting the background signal from each image, using the ImageJ built-in “subtract background” function with a default rolling ball radius of 50 pixel. The cargo signal (integrated density) in the red channel was measured and normalized to the number of nuclei counted in each field. The latter were obtained by thresholding the nuclear channel signal using the “Yen” autothreshold algorithm.

For particle analysis, the cytosolic and F-actin/membrane signals were merged into single RGB image, converted to grayscale and then thresholded to create a cellular mask of objects in the field of view, as calculated by the “Li” autothreshold algorithm. All particles in the cytosolic/membrane mask were analyzed and characteristic parameters of the particle were calculated and saved. Particles of interest were identified by filtering by area and circularity. The area was restricted to be greater than 0.78 μm² (which correspond to spheres with minimum diameter of 1 μm). Gigasomes were identified by re-directing the cytosolic mask to the thresholded nuclear and cargo channels for analysis of respective signals within gigasomes. Particles of interest were identified as gigasomes when the nuclear integrated density signal was equal zero. Cargo-positive gigasomes were identified from the particles of interest by thresholding the background-subtracted cargo channel using the “default” autothreshold algorithm. The quantity or distribution of objects obtained from the particle analysis was divided by the number of cells counted in each field to obtain a normalized quantity or distribution. The normalized quantity or distribution of objects was described as a quantity or distribution of objects per 1000 cells.

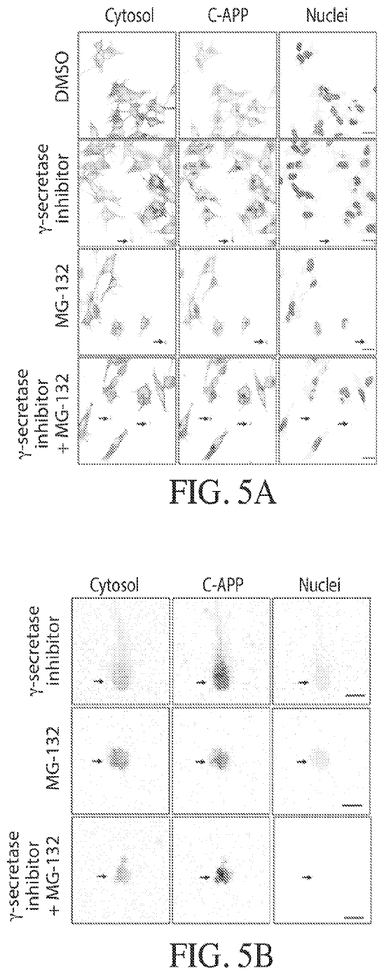

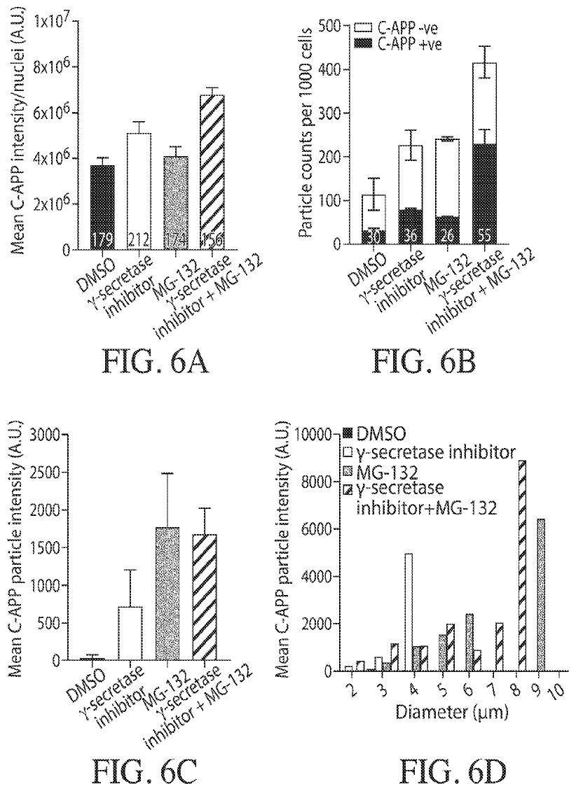

Neuronal cells treated with the γ-secretase inhibitor, either alone or in combination with MG-132, displayed increase intracellular APP-CTF levels, compared to DMSO- and MG-132-treated cells, as detected by increased C-APP antibody immunoreactivity (FIG. 5A, and respective quantification in FIG. 6A). Except for the vehicular DMSO control, the C-APP signal was detected across the treatment groups within gigasomes characterized by neuronal cytoplasmic fluorescence and absence of nuclear marker signal (FIG. 5A, arrows and enlarged FIG. 5B).

Analysis of gigasome production (FIG. 6B) from fixed plates, showed that cells incubated with DMSO produced 75-150 gigasomes per 1000 cells, whereas cells incubated with 5 mM γ-secretase inhibitor or 0.1 mM MG-132, produced 200-250 gigasomes per 1000 cells. The condition that produced the highest number of gigasomes at 400-420 gigasomes per 1000 cells was the combination of γ-secretase inhibitor and MG-132. Analysis of the C-APP+ve cargo content revealed a specific extraction of disease-related cargo by gigasome in the combination treatment with more gigasomes (>50% of the total) containing APP-CTFs (FIG. 6B). The average intensity of APP-CTF within gigasomes was assessed in different treatment groups, which showed similarly elevated levels of C-APP signal, compared to the DMSO group (FIG. 6C). C-APP+ve particle intensity distribution analysis was also assessed (FIG. 6D), which showed that gigasomes produced by MG-132 alone, and especially in combination with the γ-secretase inhibitor, were larger in diameter and brighter, as compared to the γ-secretase inhibitor and DMSO conditions (FIG. 6D).

b. Gigasome-Mediated Extraction of Stress Granule-Associated Protein HuR Following Proteasome Inhibition, in the Absence or Presence of Sodium Arsenite

In this example, neuronal cells were cultured in poly-L-lysine-coated 24-well glass bottom plates at 42,000 cells/cm² and allowed to adhere overnight, similar to methods described above in Example 13a. For this paradigm, cells were treated with a low sodium arsenite concentration of 5 μM either alone or in combination with the proteasome inhibitor MG-132 at 0.2 μM. A vehicular control amount of DMSO was added to the media to comparatively study cargo signals within cells and gigasomes. Treatments were carried out in reduced serum-containing media for 24 hours to mimic a chronic exposure to arsenite. The presence of the stress-granule-related cargo within cells and in gigasomes was characterized and quantified by confocal microscopy analysis of 4% paraformaldehyde. Non-specific labeling was blocked by incubation with 5% normal goat serum in PBS in the presence of 0.1% Triton X-100, for 2 h at room temperature, followed by immunolabeling with an antibody against the RNA-binding protein HuR (see Table 12) and incubation with Alexa Fluor® 568 secondary antibodies. Phalloidin Alexa Fluor® 488 was used to mark the proximal plasma membrane. Nuclei were counterstained with DAPI.

Images were captured using a Zeiss LSM 900 confocal microscope and a 40× objective. For analysis, 10×16-bit images per well were processed to obtain a final total range of 75-200 cells. Composite images were separated in the different channels. The background was subtracted from the red cargo-related channel, as described in Example 13a. The nuclear signal in the blue channel was thresholded using the “Yen” autothreshold algorithm. To measure the nuclear-to-cytosolic translocation of the stress-granule-related protein cargo upon treatment, firstly, the cargo-related nuclear signal was subtracted from the respective red channel image by creating a nuclear selection from the thresholded blue channel image, which was added to ImageJ Manager. The nuclear selection was then transferred to the background-subtracted red channel and the nuclear cargo signal was removed from the image leaving only the cytosolic signal. The cleared red channel image was thresholded using the “default” autothreshold algorithm for downstream particle analysis. To measure the cytosolic cargo signal, a cellular mask was then generated by thresholding the Green/F-actin channel using the “Li” autothreshold method. A cytosolic selection was created, added to ImageJ Manager, and transferred to the background subtracted, nuclear-cleared red channel image. The cytosolic cargo signal (integrated density) was measured and normalized to the number of nuclei counted in each field. Particle analysis was performed as described in Example 13a, using the thresholded green channel to identify particles of interest by filtering by area and circularity and re-directing to the blue and red channel for absence of nuclear signal and eventual presence of disease-related cargo, respectively. The quantity or distribution of objects obtained from the particle analysis was obtained as described in Example 13a.

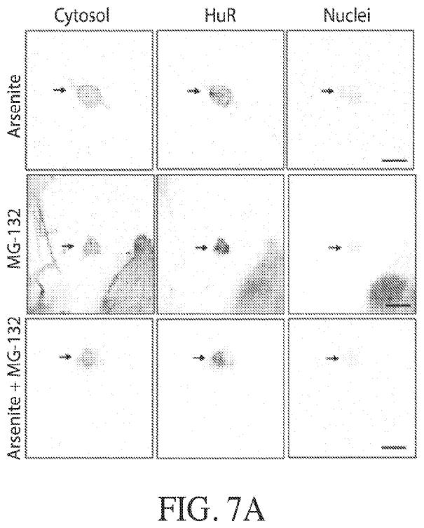

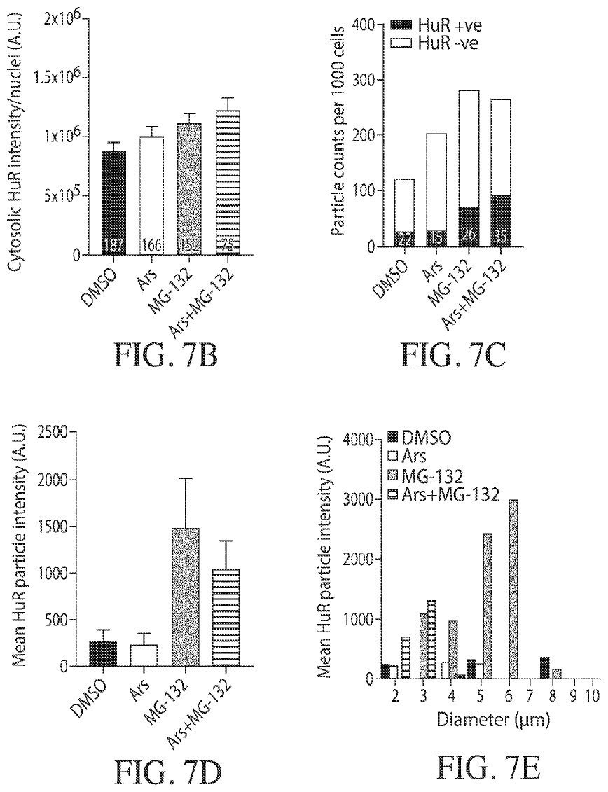

Neuronal cell treatment with 5 μM sodium arsenite for 24 hours, either alone or in combination with 0.2 μM MG-132, resulted in the generation of cytosol-positive, nuclear-negative gigasomes containing the stress granule-associated protein HuR (FIG. 7A, arrows). Normally localized in the nucleus, HuR cytosolic signal increased following sodium arsenite, MG-132 and sodium arsenite+MG-132 treatment, compared to the DMSO control condition, indicating nuclear-to-cytosolic protein translocation (FIG. 7B).

Analysis of gigasome production (FIG. 7C) from fixed plates showed that cells incubated with DMSO produced approximately 120 gigasomes per 1000 cells, whereas cells incubated with 5 μM sodium arsenite produced 200 gigasomes per 1000 cells. Cells incubated with MG-132 condition alone or combination sodium arsenite+MG-132 produced 250-300 gigasomes per 1000 cells. Gigasomes containing HuR also showed a similar rate of disease-related cargo extraction (25-35% of the total), demonstrating no further increase in gigasome production when the proteosome is inhibited in addition to the sodium arsenite treatment (FIG. 7C). HuR average intensity within gigasomes was assessed in different treatment groups, which showed higher HuR signal in the MG-132 and sodium arsenite+MG-132 combination treatment, compared to sodium arsenite alone and DMSO (FIG. 7D). HuR+ve particle intensity distribution analysis (FIG. 7E) showed that gigasomes produced by MG-132 alone, but not MG-132+sodium arsenite, were larger in diameter and brighter (FIG. 7B, 7C).

These results show that the stress-granule-associated protein HuR can be extracted by gigasomes under conditions that promote stress granule formation or proteasome inhibition. However, proteosome inhibition combined with stress granule formation did not further increase gigasome production nor gigasome-mediated extraction of HuR.

c. Tau Fibrils Exogenously Added to Neuronal Cell Culture Media are Rapidly Taken Up by Cells and Subsequently Extracted by Gigasomes in the Absence or Presence of Proteasome Inhibition

In this example, neuronal cells were cultured in 24-well glass bottom plates (uncoated) at 42,000 cells/cm² and allowed to adhere overnight. P301S mutant Tau fibrils conjugated to the fluorescent probe ATTO 488 (Table 12) were resuspended at 50 nM in reduced serum-containing media and added to half of the cell-containing wells for 3 hours at 37° C. Following Tau fibrils uptake, cells were trypsinized to remove excess Tau fibrils attached to the cell surface, labeled with Hoechst 33342 to visualize nuclei and CellTracker Orange to visualize the cytoplasm and plated in a new poly-L-lysine-coated 24-well glass bottom plates. Cells were allowed to attach (2-3 hours), before addition of 0.1 μM MG-132 in reduced serum-containing media for additional 18-20 h. Four treatment group were analyzed for gigasome production and extraction of Tau fibrils by gigasomes: 1) cells that did not receive Tau fibrils and treated with DMSO (DMSO); 2) cells that did receive Tau fibrils and treated with DMSO (Tau-F); 3) cells that did not receive Tau fibrils and treated with MG-132 proteosome inhibitor (MG-132); and 4) cells that did receive Tau fibrils and treated with MG-132 proteosome inhibitor (Tau-F+MG-132).

Live cells images were taken using a Zeiss LSM 900 confocal microscope 18-20 hours following the addition of MG-132. For analysis, 10-15×16-bit images per well were captured with a 20× objective and processed for analysis of gigasome production. Composite images were separated in the different channels. The background was subtracted from the green Tau fibrils cargo channel, as described Example 13.a and the signal thresholded using the “Yen” autothreshold algorithm. The cargo signal (integrated density) in the green channel was measured and normalized to the number of nuclei counted in each field in the Tau-F conditions. The latter were obtained by thresholding the nuclear channel signal using the “Otsu” autothreshold algorithm followed by a watershed binary segmentation to separate grouped nuclei. The red cytosolic channel was thresholded using the “Li” autothreshold algorithm and particle analysis carried out as described in Example 13a. The quantity or distribution of objects obtained from the particle analysis was divided by the number of cells counted in each field to obtain a normalized quantity or distribution. The normalized quantity or distribution of objects was described as a quantity or distribution of objects per 1000 cells. At the end of the treatment gigasomes were also harvested from culture media supernatants and processed for analysis as described in Example 2.

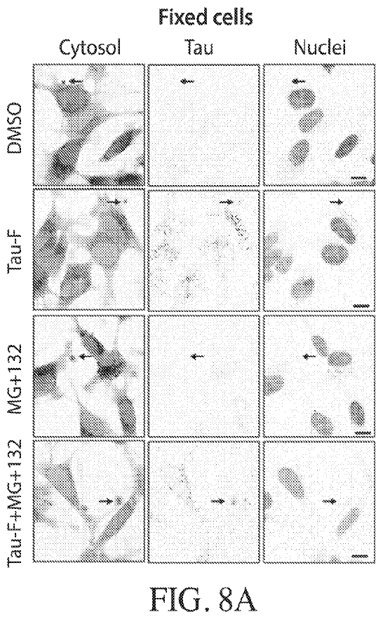

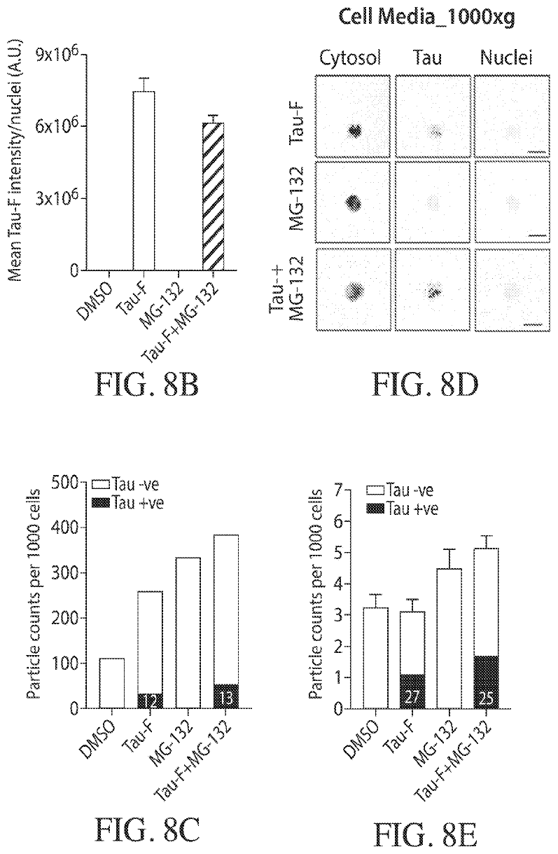

Tau fibrils were efficiently taken up by neuronal cultures and a bright punctate intracellular staining was still detected following 24 hours in culture with no difference in signal intensity between Tau-F and Tau-F+MG-132 conditions (FIGS. 8A and 8B). Cytosolic marker-positive, nuclear marker-negative gigasomes (FIG. 8A, arrows) were detected in all treatment groups and only cells in the Tau-F and Tau-F+MG-132 conditions generated gigasomes containing Tau fibrils.

Analysis of gigasome production (FIG. 8C) from plated cells showed that the DMSO condition produced 100-120 gigasomes per 1000 cells, Tau-F condition produced 250 gigasomes per 1000 cells, MG-132 condition produced approximately 325 gigasomes per 1000 cells, and Tau-F+MG-132 condition produced approximately 375-400 gigasomes per 1000 cell. Approximately 12% and 13% of gigasomes contained Tau fibrils cargo in the Tau-F and Tau-F+MG-132 conditions, respectively (FIG. 8C).

Separately, gigasomes were also harvested from the media and analyzed. Gigasomes harvested from the cell culture media of Tau-F and Tau-F+MG-132 conditions expressed bright Tau-F particles, compared to gigasomes harvested from the MG-132 condition which did not have any notable expression (FIG. 8D). Analysis of these gigasomes in the media revealed only approximately 3 gigasomes per 1000 cells in the DMSO and Tau-F conditions, approximately 4.5 gigasomes per 1000 cells in the MG-132 condition, and approximately 5-5.5 gigasomes per 1000 cells in the Tau-F+MG-132 condition (FIG. 8E). Approximately 27% and 25% of gigasomes contained Tau fibrils cargo in the Tau-F and Tau-F+MG-132 conditions, respectively (FIG. 8E).

These results show that Tau-F internalized by the cells can be extracted by gigasomes, with proteosome inhibition further promoting gigasome-mediated extraction.

### Example 14: Visualization Gigasome Generation Process and Characterization of Gigasome Cargo

### Quantification of Organelle Cargo of Neuronal Gigasomes Using Fluorescence Microscopy

Live neuronal cells were cultured in 24-well glass bottom plates at 42,000 cells/cm² and allowed to adhere overnight. Cells were incubated with Hoechst 33342 to stain the nucleus and either CellTracker Green or CellTracker Orange to stain the cytosol. Cells were also incubated with an organelle cargo stain or marker from Table 13. Gigasome production was induced after washing the stains by incubating the cells with 10 nM MG-132 and 31 nM Bafilomycin-A1 for 24 hours. Cell culture media supernatant samples were centrifuged at 300× g for 5 minutes at 4° C. The supernatant was then transferred into a new tube and centrifuged at 1000× g for 5 minutes at 4° C. The enriched gigasome pellets in the 1000× g spins were washed with buffer (PBS) before being resuspended in 100 μL of PBS and plated in a 96-well plate for confocal microscopy.

TABLE 13
Stains and transfection markers for organelle gigasome cargo mapping

| Stain/Marker | Type | Organelle | Target |
|---|---|---|---|
| Mito Tracker Deep Red | Stain | Mitochondria | Thiol-reactive chloromethyl |
| | | | groups |
| Lyso Tracker Deep Red | Stain | Lysosome | Acidic vesicle by protonation |
| BOPIDY TR Ceramide | Stain | Golgi Apparatus | Sphingolipid accumulation |
| CellMask Deep Red | Stain | Plasma Membrane | Lipids |
| CellLight | Transfection | Mitochondria | E1 alpha pyruvate |
| Mitochondria-RFP | | | dehydrogenase |
| CellLight Actin-RFP | Transfection | Actin | Human Actin |
| CellLight Tubulin-RFP | Transfection | Tubulin | Human Tubulin |
| CellLight ER-RFP | Transfection | Endoplasmic Reticulum | Calreticulin and KDEL |
| CellLight Late | Transfection | Late Endosomes | Rab7a |
| Endosomes-RFP | | | |
| CellLight Peroxisomes- | Transfection | Peroxisomes | Peroxisomal C-terminal |
| GFP | | | |

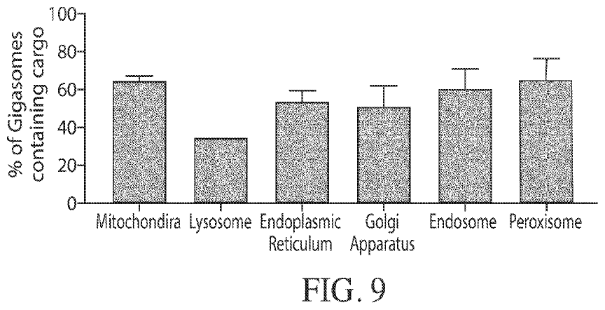

The Zeiss ZEN Blue software was used to distribute imaging fields of view (positions) to create an unbiased imaging sample of a well. Images were taken using a Zeiss LSM 900 confocal microscope. Images were in 16-bit format, and contained at least an ESID (brightfield) channel, a Hoechst 33342 fluorescence channel, a Cytosolic fluorescence channel, and an Organelle Cargo fluorescence channel. In some experiments, 15 images per well were taken. ImageJ was used to impose a threshold to create a mask of objects in the field of view. The Cytosolic fluorescence channel was thresholded between 5403 and 65535 for 16-bit images. A watershed binary segmentation step was performed to separate grouped objects. For each particle in the mask, characteristic parameters of the particle were calculated and saved. In some experiments, these parameters were the particles area, circularity, mean fluorescence signal in the Hoechst 33342 channel (nuclear signal), and mean fluorescence signal in the Organelle Cargo channel. Particles of interest were identified by thresholding by area and circularity. In some experiments, area was restricted to be between 0.78 and 314 μm² (which correspond to spheres with diameters ranging from 1 to 20 μm). Gigasomes were identified from the particles of interest by thresholding by mean nuclear signal. In some experiments, nuclear signal threshold was set using a value equal to half of a threshold value calculated by the Li auto-threshold algorithm applied to the distribution of all particles of interest. Particles of interest with nuclear signal below this value were considered “negative” for nuclear material and were identified as gigasomes. Gigasomes were further analyzed by Organell Cargo signal. Cargo-positive gigasomes were identified from the particles of interest by thresholding by Organell Cargo signal. A threshold was set by considering the background fluorescence signal from an unstained control population of gigasomes. Gigasomes with an Organelle Cargo mean value above the fluorescence threshold were considered positive for the respective organelle. For organelles targeted by temporary transfection, the percent of gigasomes containing the organelle was divided by the estimated transfection efficiency of the cells in the well, which corrected for false negatives from gigasomes that did contain the organelles but came from non-transfected cells. Analysis of fluorescence microscopy images revealed that neuronal gigasomes contain a diversity of large cellular cargo, including mitochondria, lysosome, endoplasmic reticulum, Golgi body, endosome, and/or peroxisome (FIG. 9).

### Example 15: Modulation and Quantification of Cardiomyocyte Gigasome Yield, Purity, and Distribution

This example shows that gigasomes can be differentially induced upon treatment with various compounds alone or in combination as stated in Table 10. It also shows that the combination of AZD2014 and BafA1 produced the highest gigasomes in cardiomyocyte cells in the conditions and compounds tested in this example.

To quantify and characterize gigasomes using microscopy, gigasomes were harvested from cell culture media supernatants. Cells were incubated with Hoechst 33342, CellTracker Green and MitoTracker Deep Red stains to detect nuclear material, cytoplasm, and mitochondria, respectively, and cultured in poly-L-lysine-coated 24-well glass bottom plates at 15,800 cells/cm² and allowed to adhere overnight. Gigasome production was then induced by addition of various compounds as described in Examples 1 and 10. In some experiments, compounds were added either alone or in combination, and a vehicular control amount of DMSO were added to the existing cell culture media to comparatively study gigasome production rates. As a control group, 500 nM Staurosporine was added to induce apoptosis and produce apoptotic bodies with nuclear material. Cultures were incubated for 24 hours under gigasome inducing conditions.

Cell culture media supernatant samples were collected, followed by a cell wash with accutase to help collecting particles attached to the plate. In some experiments cells were washed with PBS buffer containing 0.5 mM EDTA. Cell culture media and cell washes were pooled and centrifuged at 300× g for 5 minutes at 4° C. The resulting supernatants were then centrifuged at 1,000× g for 5 minutes at 4° C., resulting in an enriched pellet of gigasomes. The resulting pellet of cells and cellular debris after the 300× g spins were saved for down-stream analysis. The enriched gigasome pellets in the 1000× g spins were washed with buffer (PBS) before being resuspended in 100 uL of PBS and plated in a 96-well plate for confocal microscopy.

The Zeiss ZEN Blue software was used to distribute imaging fields of view (positions) to create an unbiased imaging sample of a well. Images were taken using a Zeiss LSM 900 confocal microscope. Images were in 16-bit format, and contained at least an ESID (brightfield) channel, a Hoechst 33342 fluorescence channel, and a MitoTracker Deep Red fluorescence channel. In some experiments, images also contained a CellTracker Green fluorescence channel. In some experiments, 15 images per well were taken. In some experiments, 10 images per well were taken for cellular pellet samples, and 30 images per well were taken for gigasome enriched pellet samples. ImageJ was used to impose a threshold to create a mask of objects in the field of view. In some experiments, the variance of the brightfield channel was calculated with a radius of 5 pixels and the threshold positive window was between 5 and 65535 for 16-bit images. In some experiments, the CellTracker Green fluorescence channel was threshold between 5403 and 65535 was set for 16-bit images. A watershed binary segmentation step was performed to separate grouped objects. For each particle in the mask, characteristic parameters of the particle were calculated and saved. In some experiments, these parameters were the particles area, circularity, mean fluorescence signal in the Hoechst 33342 channel (nuclear signal), and mean fluorescence signal in the MitoTracker Deep Red channel (mitochondrial signal).

Particles of interest were identified by thresholding by area and circularity. In some experiments, area was restricted to be between 0.78 and 314 μm² (which correspond to spheres with diameters ranging from 1 to 20 μm). Gigasomes were identified from the particles of interest by thresholding by mean nuclear signal. In some experiments, nuclear signal threshold was set using a value equal to half of a threshold value calculated by the Li auto-threshold algorithm applied to the distribution of all particles of interest. Particles of interest with nuclear signal below this value were considered “negative” for nuclear material and were identified as gigasomes. In some experiments, gigasomes were further analyzed by mitochondrial signal.

In some experiments, a sampling factor was calculated to extrapolate the total quantity of a type of object (particles, particles of interest, gigasomes, or mitochondrial gigasomes) in the well. The sampling factor was the percentage of the well's area that was imaged. Dividing a quantity or distribution of objects by the sampling factor resulted in an estimated quantity or distribution of objects for the whole well. In some experiments, the number of cells in the cell cultures were estimated with a cell counter. Dividing a quantity or distribution of objects by the number of cells from the culture that produced them resulted in a normalized quantity or distribution. In some experiments, the normalized quantity or distribution of objects was described as a quantity or distribution of objects per 1000 cells.

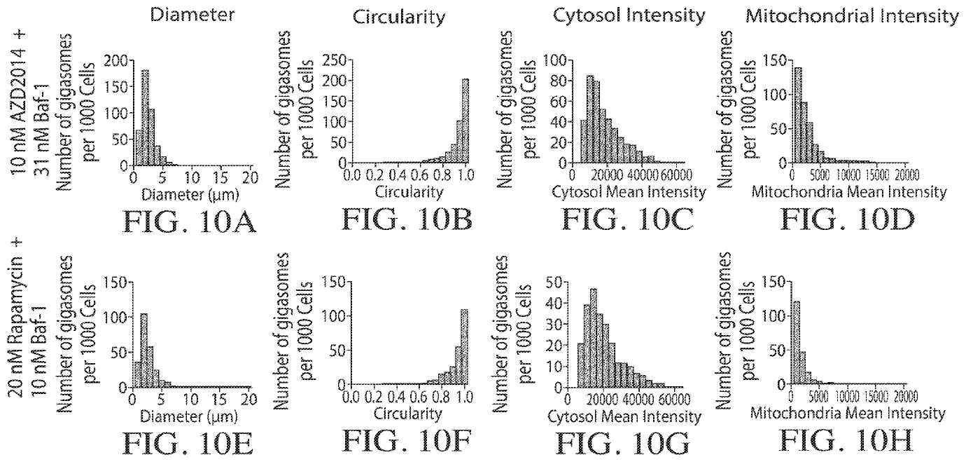

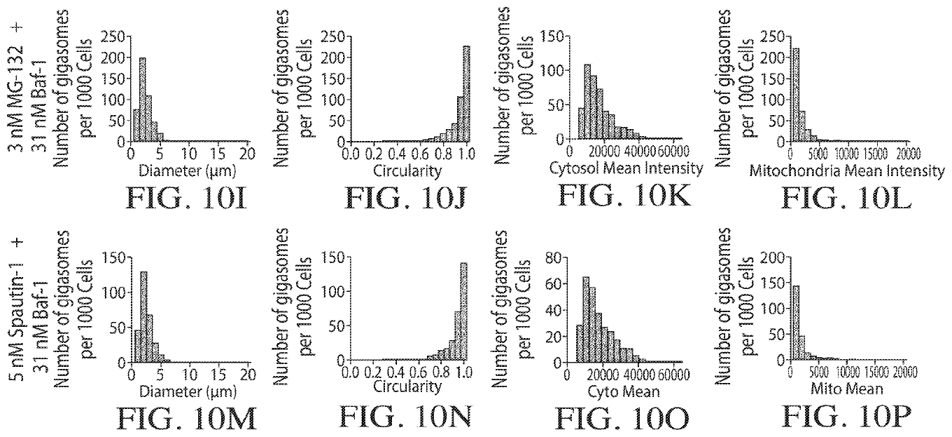

To characterize the gigasomes produced by the various compounds, different parameters of the enriched gigasomes were quantified and plotted (FIG. 10A-10P). In this example, the characteristics of the gigasomes produced by cardiomyocyte cells treated with 10 nM AZD2014 in combination with 31 nM BafA1 (FIG. 10A-10D), 20 nM Rapamycin in combination with 10 nM BafA1 (FIG. 10E-10H), 3 nM MG-132 in combination with 31 nM BafA (FIG. 10I-10L), and 5 nM Spautin-1 in combination with 31 nM BafA FIG. 10M-10P) were analyzed. The size analytics of the gigasomes produced showed a similar distribution irrespective of the compound treatment (FIG. 10A, 10E, 10I, 10M), even though the number of gigasomes produced differed between treatments. Similarly, the distribution for the circularity of the gigasomes (FIG. 10B, 10F, 10J, 10N), the cytoplasmic content (FIG. 10C, 10G, 10K, 10O) and the mitochondrial intensity (FIG. 10D, 10H, 10L, 10P) were similar across treatments.

Cardiomyocyte cells under different drug conditions either alone or in combination displayed differential rates of gigasome production (Table 14). For example, a combination of cells incubated with 20 nM Rapamycin and 31 nM BafA1 produced between 300-400 gigasomes per 1000 cells, whereas cells incubated with Spautin-1 at 51 nM and BafA1 at 1 nM produced fewer than 50 gigasomes per 1000 cells. Various combination of AZD214, Rapamycin and MG-132 with BafA1 at 10 nM and 31 nM produced higher than 200 gigasomes per 1000 cells (Table 14). Analysis of the percentages of a nuclear material produced by the cells revealed enrichment of the gigasomes in some conditions (>75%) using the differential centrifugation protocol as stated in Example 2. The viability of the parent cells 24 hours after the various drug treatments (as compared to the control DMSO condition) was also assessed using Cell Titer Glo assay (Table 14). In the conditions and compounds tested in this example, the combination of AZD2015 and BafA1 produced the highest number gigasomes in cardiomyocyte cells (Table 14).

TABLE 14
Quantification and characterization of gigasomes enriched from cell culture
media from cardiomyocyte cells treated with various compounds.

| | | Particles | |
|---|---|---|---|
| Compound name | No. of gigasomes | without | |
| (Concentration in nM) | per 1000 cells | nucleus (%) | % Viability |
| DMSO | 50-100 | >75 | 100 |
| AZD2014 (10) + BafilomycinA1 (0.1) | 50-100 | >75 | >75 |
| AZD2014 (10) + BafilomycinA1 (1) | 50-100 | >75 | >75 |
| AZD2014 (10) + BafilomycinA1 (10) | 200-300 | >75 | >75 |
| AZD2014 (10) + BafilomycinA1 (31) | 400-500 | >75 | >75 |
| AZD2014 (31) + BafilomycinA1 (0.1) | 50-100 | >75 | >75 |
| AZD2014 (31) + BafilomycinA1 (1) | 50-100 | >75 | >75 |
| AZD2014 (31) + BafilomycinA1 (10) | 200-300 | >75 | >75 |
| AZD2014 (31) + BafilomycinA1 (31) | 400-500 | >75 | >75 |
| AZD2014 (100) + BafilomycinA1 (0.1) | 50-100 | >75 | >75 |
| AZD2014 (100) + BafilomycinA1 (1) | 100-200 | >75 | >75 |
| AZD2014 (100) + BafilomycinA1 (10) | 200-300 | >75 | >75 |
| AZD2014 (100) + BafilomycinA1 (31) | >500 | >75 | >75 |
| Rapamycin (6) + BafilomycinA1 (0.1) | 100-200 | >75 | >75 |
| Rapamycin (6) + BafilomycinA1 (1) | 50-100 | >75 | >75 |
| Rapamycin (6) + BafilomycinA1 (10) | 200-300 | >75 | 50-75 |
| Rapamycin (6) + BafilomycinA1 (31) | 400-500 | >75 | 50-75 |
| Rapamycin (20) + BafilomycinA1 (0.1) | 50-100 | >75 | >75 |
| Rapamycin (20) + BafilomycinA1 (1) | 50-100 | >75 | >75 |
| Rapamycin (20) + BafilomycinA1 (10) | 200-300 | >75 | 50-75 |
| Rapamycin (20) + BafilomycinA1 (31) | 300-400 | >75 | 50-75 |
| Rapamycin (60) + BafilomycinA1 (0.1) | 50-100 | >75 | >75 |
| Rapamycin (60) + BafilomycinA1 (1) | 50-100 | >75 | >75 |
| Rapamycin (60) + BafilomycinA1 (10) | 200-300 | >75 | >75 |
| Rapamycin (60) + BafilomycinA1 (31) | >500 | >75 | 50-75 |
| MG-132 (3) + BafilomycinA1 (0.1) | 50-100 | >75 | >75 |
| MG-132 (3) + BafilomycinA1 (1) | 50-100 | >75 | >75 |
| MG-132 (3) + BafilomycinA1 (10) | 200-300 | >75 | >75 |
| MG-132 (3) + BafilomycinA1 (31) | 400-500 | >75 | >75 |
| MG-132 (10) + BafilomycinA1 (0.1) | 50-100 | >75 | >75 |
| MG-132 (10) + BafilomycinA1 (1) | 50-100 | >75 | >75 |
| MG-132 (10) + BafilomycinA1 (10) | 200-300 | >75 | >75 |
| MG-132 (10) + BafilomycinA1 (31) | 200-300 | >75 | >75 |
| MG-132 (31) + BafilomycinA1 (0.1) | 50-100 | >75 | >75 |
| MG-132 (31) + BafilomycinA1 (1) | 50-100 | >75 | >75 |
| MG-132 (31) + BafilomycinA1 (10) | 300-400 | >75 | >75 |
| MG-132 (31) + BafilomycinA1 (31) | 400-500 | >75 | >75 |
| Spautin-1 (5) + BafilomycinA1 (0.1) | 50-100 | >75 | >75 |
| Spautin-1 (5) + BafilomycinA1 (1) | 50-100 | >75 | >75 |
| Spautin-1 (5) + BafilomycinA1 (10) | 50-100 | >75 | >75 |
| Spautin-1 (5) + BafilomycinA1 (31) | 50-100 | >75 | >75 |
| Spautin-1 (50) + BafilomycinA1 (0.1) | 50-100 | >75 | >75 |
| Spautin-1 (50) + BafilomycinA1 (1) | <50 | >75 | >75 |
| Spautin-1 (50) + BafilomycinA1 (10) | 50-100 | >75 | >75 |
| Spautin-1 (50) + BafilomycinA1 (31) | 100-200 | >75 | >75 |
| Spautin-1 (500) + BafilomycinA1 (0.1) | 100-200 | >75 | >75 |
| Spautin-1 (500) + BafilomycinA1 (1) | 200-300 | >75 | >75 |
| Spautin-1 (500) + BafilomycinA1 (10) | 200-300 | >75 | >75 |
| Spautin-1 (500) + BafilomycinA1 (31) | 200-300 | >75 | >75 |

### Example 16: Proteomic Analyses of the Gigasomes Using Mass Spectrometry (MS)

In this example, the proteomic composition of neuronal gigasomes were characterized and contrasted with apoptotic bodies. This example reveals the distinction between gigasomes and apoptotic bodies, and aids in identification of unique gigasome biomarkers and pathways.

### Collection of Gigasomes and Apoptotic Bodies Produced by Drug Induction in Neuronal Monocultures

Neuronal cells were cultured in T175 flasks at a seeding density of 42,000 cells/cm². To induce gigasome production, cells were treated with specified compounds that induce gigasomes and compounds that induce apoptosis for 24 hours. Gigasomes enriched from cells treated with 10 nM MG-132+31 nM Bafilomycin A1 are referred to as Gigasome Group A; Gigasomes enriched from cells treated with 100 nM MG-132 are referred to as Gigasome Group B; Gigasomes enriched from cells treated with 31 nM Bafilomycin A1 are referred to as Gigasome Group C. Apoptotic bodies enriched from cells treated with 500 nM Staurosporine to induce apoptosis are referred to as Apoptotic bodies. Five culture flasks were grown per condition and combined to collect sufficient gigasomes for mass spectrometric analyses. For apoptotic bodies, two flasks per condition were combined. Each condition was analyzed in triplicate except for Gigasome Group C, which was performed in duplicate.

To harvest gigasomes produced by the cells after appropriate treatments, cell culture media was transferred from cell culture plates into 15 mL conical tubes. The cells were washed with 3× ice-cold PBS containing MS-safe Protease and Phosphatase inhibitors (PBS-PI) and the washes were collected in the conical tubes as well. The samples were then centrifuged at 50× g for 5 minutes at 4° C. The resulting supernatant was transferred to a new tube, and the pellet of cells was stored for future analysis. These supernatants were centrifuged at 300× g for 5 minutes at 4° C. to further remove any cell debris. The supernatant was transferred to new tubes. These samples were further centrifuged at 1,000× g for 5 minutes at 4° C. The supernatants were discarded and the resulting pellet of enriched gigasomes (or apoptotic bodies) were consolidated into a single conical tube. These pellets were washed twice by resuspending in ice-cold PBS-PI and spinning down at 1,000× g for 5 minutes at 4° C. The washed pellets were then resuspended in ˜50 ul of PBS-PI and transferred onto 2 ml Lo-Bind tubes to prevent any loss of protein due to binding to the tubes. These enriched gigasomes and apoptotic bodies were snap-frozen in dry-ice and stored at −80° C., until ready to be analyzed by mass spectrometry.

### Protein Extraction and Digestion

Proteins were denatured in 1% sodium dodecyl sulfate (w/v) and reduced with 20 mM dithiothreitol (DTT) for 1 hour at room temperature. Cysteine residues were alkylated with iodoacetamide (60 mM) for 1 hour in the dark and quenched with DTT (40 mM). Proteins were extracted by methanol-chloroform precipitation and digested with 1 μg of trypsin (Promega) in 100 mM EPPS (pH 8.0) for 4 hours at 37° C. Each of the tryptic peptide samples were labeled with 200 μg of Tandem Mass Tag (TMT; Pierce) isobaric reagents for 2 hours at room temperature. A label efficiency check was performed by pooling 2 μL from each sample within a single plex to ensure at least 98% labeling of all N-termini and lysine residues. All samples were quenched with hydroxylamine (0.5%), acidified with TFA (2%), pooled, and dried by speedvac evaporation. The dried pooled TMT labeled peptides were resuspended in 0.1% TFA. and subjected to orthogonal basic-pH reverse phase fractionation on a 2.1×50 mm column packed with 1.8 m ZORBAX Extend-C18 material (Agilent, Santa Clara, CA) equilibrated with buffer A (5% acetonitrile in 10 mM ammonium bicarbonate, pH 8). Peptides were fractionated utilizing a 4 min linear gradient from 5% to 50% buffer B (90% acetonitrile in 10 mM ammonium bicarbonate, pH 8) at a flow rate of 0.3 mL/min. Six fractions were consolidated into 3 samples and vacuum dried. The samples were resuspended in 0.1% TFA desalted on StageTips and vacuum dried.

### Mass Spectrometry Analysis of Gigasomes and Apoptotic Bodies

All mass spectra were acquired on an Orbitrap Fusion Lumos coupled to an EASY nanoLC-1200 (ThermoFisher) liquid chromatography system. Approximately 2 μg of peptides were loaded on a 75 μm capillary column packed in-house with Sepax GP-C18 resin (1.8 μm, 150 Å, Sepax) to a final length of 35 cm. Peptides were separated using an 80-minute linear gradient from 8% to 28% acetonitrile in 0.1% formic acid. The mass spectrometer was operated in a data dependent mode. The scan sequence began with FTMS1 spectra (resolution=120,000; mass range of 350-1400 m/z; max injection time of 50 ms; AGC target of 1e6; dynamic exclusion for 60 seconds with a +/−10 ppm window). The 10 most intense precursors were selected within a 2 s cycle and fragmented via collisional-induced dissociation (CID) in the ion trap (normalized collision energy (NCE)=35; max injection time=35 ms; isolation window of 0.7 Da; AGC target of 1e4). Following ITMS2 acquisition, a real-time search (RTS) algorithm was employed to score each peptide and only trigger synchronous-precursor-selection (SPS) MS3 quantitative spectra for high confidence scoring peptides as determined by a linear discriminant approach. Following MS2 acquisition, a synchronous-precursor-selection (SPS) MS3 method was enabled to select eight MS2 product ions for high energy collisional-induced dissociation (HCD) with analysis in the Orbitrap (NCE=55; resolution=50,000; max injection time=86 ms; AGC target of 1.4e5; isolation window at 1.2 Da for +2 m/z, 1.0 Da for +3 m/z or 0.8 Da for +4 to +6 m/z). All mass spectra were converted to mzXML using a modified version of ReAdW.exe. MS/MS spectra were searched against a concatenated 2021 human Uniprot protein database containing common contaminants (forward+reverse sequences) using the SEQUEST algorithm (Eng et al., 1994).

Database search criteria are as follows: fully tryptic with two missed cleavages; a precursor mass tolerance of 50 ppm and a fragment ion tolerance of 1 Da; oxidation of methionine (15.9949 Da) was set as differential modifications. Static modifications were carboxyamidomethylation of cysteines (57.0214) and TMT on lysines and N-termini of peptides (229.1629). Peptide-spectrum matches were filtered using linear discriminant analysis (Huttlin et al, Cell 2010) and adjusted to a 1% peptide false discovery rate (FDR) (Elias et al, Nat Methods 2007) and collapsed further to a final 1.0% protein-level FDR. Proteins were quantified by summing the total reporter intensities across all matching PSMs, hereon referred to as raw counts.

### Mass Spectrometry Data Analysis

For MS data analysis, the software Spectromine, v3.2 was used, and proteins were identified using the UniProt human Database. Samples were 11-plexed and TMT-tagged, 3875 protein groups were identified and were further analyzed to compare protein expression levels between gigasomes and apoptotic bodies.

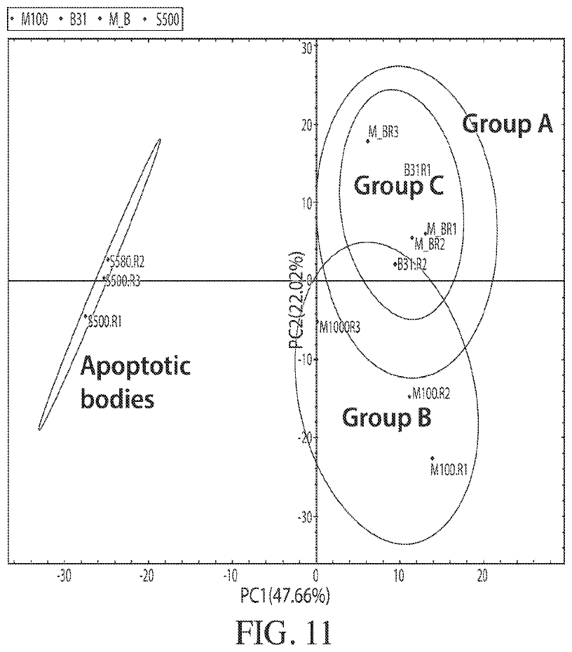

Principal component analysis (PCA) on these protein groups was performed to assess the distribution of these proteins. Proteins expressed in apoptotic bodies clustered very differently and did not overlap with any of the three gigasome groups (FIG. 11). This suggests a unique proteomic signature and expression profile of proteins in the gigasomes as compared to the apoptotic bodies. On the other hand, there were significant overlaps between the three gigasome groups assessed (FIG. 11), suggesting common pathways or proteomic profiles.

To further characterize the proteins identified, proteins were compared between each gigasome group and apoptotic bodies. Significance was calculated as a hyperbola score which equals −log(p-value)*log(2)fold-of-change, where the p-value is <0.05. Therefore, a hyperbola score of >2.6 for upregulated proteins and <−2.6 for downregulated proteins was considered significant. Similar to the PCA analysis in FIG. 11, the quantity of differentially expressed proteins (<200) within the three Gigasome Groups was less than the quantity of differentially expressed proteins (>200) between the Gigasome Groups and apoptotic bodies (Table 15). This further suggests unique proteome signatures in gigasomes as compared to apoptotic bodies.

TABLE 15
Total number of significantly upregulated and downregulated
proteins when compared within each group. 3 groups of gigasomes -
Group A, B, C and Apoptotic bodies. Significance was determined
using Hyperbola score. Hyperbola score = −log(p-value) *
log(2)fold-of-change. p-value < 0.05.

| | No. of | No. of |
|---|---|---|
| Pairwise comparisons | Upregulated proteins | Downregulated proteins |
| A vs B | 79 | 181 |
| A vs C | 1 | 0 |
| B vs C | 37 | 165 |
| A vs Apoptotic bodies | 486 | 515 |
| B vs Apoptotic bodies | 494 | 274 |
| C vs Apoptotic bodies | 468 | 405 |

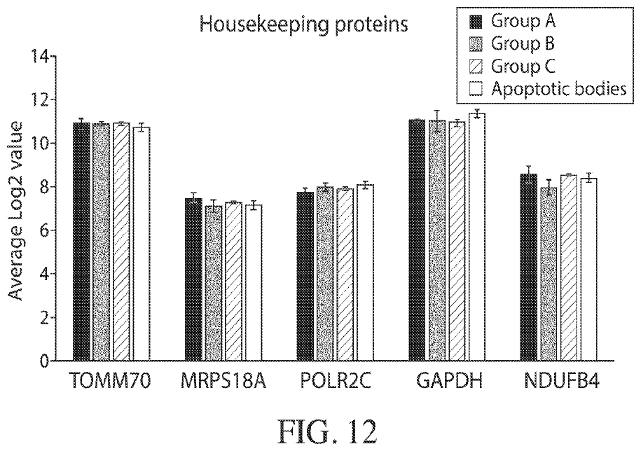

To further analyze the proteins identified, protein levels were first normalized to those of five housekeeping proteins. The housekeeping proteins were chosen such that they would express at a constant and stable level across the all groups (Caracausi et al, Mol Med Rep 2017). The housekeeping proteins chosen were TOMM70 (Mitochondrial import receptor subunit 70), MRPS18A (39S ribosomal protein S18a), POLR2C (DNA-directed RNA polymerase II subunit RPB3), GAPDH (Glyceraldehyde-3-phosphate dehydrogenase) and NDUFB4 (NADH dehydrogenase 1 beta subcomplex subunit 4) (FIG. 12). In this dataset, the log 2 values of the raw counts were calculated for each group, averaged within replicates, and then compared to assess similarities in abundance within the gigasome groups and apoptotic bodies (FIG. 12, Tables 16 and 18). In some cases, the averages of all five of the housekeeping protein raw counts were calculated, in order to directly normalize each protein raw count value to the housekeeping protein raw count values (Tables 17 and 19). Each group displayed similar values of the housekeeping proteins suggesting that the treatments to induce gigasome production or apoptotic bodies did not affect the expression levels of these proteins (FIG. 12), and thus could be utilized for normalization of other proteins.

To identify upregulated proteins in the gigasomes as compared to the apoptotic bodies, first the raw values of all the proteins were transformed using log 2. Next, the log 2 transformed values of every protein was normalized to the average log 2 values of five housekeeping proteins identified in FIG. 12. This normalization was performed first, by calculating the average of the five housekeeping proteins across each group. Next, the deviation for each group from the average was calculated by subtracting the average across all groups from the average of each group. The resulting deviation per group were then subtracted from the log 2 values of each protein in that group. These log 2 values were then averaged across replicates per group. The fold changes of each protein in the gigasome groups compared to the apoptotic bodies were calculated by using the formula:

$$Log 2 Fold Change ( Log 2 FC ) = {X}_{A} - {X}_{AB}$$

Where X_{A}=Averaged normalized log 2 transformed counts of a specific protein from a gigasome group
X_{AB}=Averaged normalized log 2 transformed counts of same protein from the apoptotic bodies group

Next, to find significantly different proteins, a p-value cut-off was established. Significances denoted by p-values were calculated using a two-tailed Student's T-test comparing two sample unequal variances (type 3) for X_{A}, X_{AB}, and a p-value of <0.05 was considered significant. Only proteins that were significantly upregulated in all the 3 gigasome groups as compared to the apoptotic bodies were considered for further analysis. Then, the resulting list of proteins were sorted based on the highest log 2 fold change in Gigasome group A in comparison to the apoptotic bodies group, followed by gigasome Group B vs apoptotic bodies and finally Gigasome C vs apoptotic bodies. Finally, only proteins that were present in all the three gigasome groups were considered. This analysis yielded 267 significantly upregulated proteins in the gigasome groups A, B and C as compared to the apoptotic bodies. Table 16 presents the top 50 proteins with the highest Log 2 fold change values after the p-value cut-off of <0.05 in the Gigasome groups A, B and C compared to Apoptotic bodies.

TABLE 16
Log2 fold change values of significantly upregulated proteins across all three
Gigasomes Groups compared to Apoptotic bodies. Proteins are sorted by order
of log2 fold change. Only proteins with p-value < 0.05 are included in table.

| | | | Gigasome | Gigasome | Gigasome |
|---|---|---|---|---|---|
| | | | Group A | Group B | Group C |
| | | | Log2 Fold Change | Log2 Fold Change | Log2 Fold Change |
| | | | from Apoptotic | from Apoptotic | from Apoptotic |
| No. | Gene ID | Protein name | bodies | bodies | bodies |
| 1 | PANX1 | Pannexin-1 | 4.185 | 2.740 | 4.237 |
| 2 | PDAP1 | 28 kDa heat- and | 4.092 | 4.103 | 3.999 |
| | | acid-stable | | | |
| | | phosphoprotein | | | |
| 3 | CIRBP | Cold-inducible | 4.081 | 4.648 | 4.021 |
| | | RNA-binding | | | |
| | | protein | | | |
| 4 | RBMX | RNA-binding motif | 3.911 | 4.020 | 3.760 |
| | | protein, X | | | |
| | | chromosome | | | |
| 5 | PTMA | Prothymosin alpha | 3.412 | 4.631 | 3.973 |
| 6 | BCL7A | Isoform 2 of B-cell | 3.336 | 3.849 | 3.202 |
| | | CLL/lymphoma 7 | | | |
| | | protein family | | | |
| | | member A | | | |
| 7 | STMN1 | Isoform 2 of | 3.318 | 3.328 | 3.340 |
| | | Stathmin | | | |
| 8 | PLTP | Phospholipid | 3.175 | 3.606 | 3.485 |
| | | transfer protein | | | |
| 9 | HMGN2 | Non-histone | 3.126 | 3.877 | 3.178 |
| | | chromosomal protein | | | |
| | | HMG-17 | | | |
| 10 | PYM1 | Partner of Y14 and | 3.102 | 3.161 | 3.122 |
| | | mago | | | |
| 11 | AKAP12 | A-kinase anchor | 3.020 | 2.579 | 3.044 |
| | | protein 12 | | | |
| 12 | CHCHD2 | Coiled-coil-helix- | 2.960 | 3.437 | 2.851 |
| | | coiled-coil-helix | | | |
| | | domain-containing | | | |
| | | protein 2 | | | |
| 13 | MDK | Midkine | 2.939 | 2.334 | 2.775 |
| 14 | TMSB10 | Thymosin beta-10 | 2.906 | 2.785 | 2.963 |
| 15 | TMA7 | Translation | 2.864 | 3.648 | 2.825 |
| | | machinery- | | | |
| | | associated protein 7 | | | |
| 16 | MRPL34 | 39S ribosomal | 2.850 | 2.996 | 2.923 |
| | | protein L34, | | | |
| | | mitochondrial | | | |
| 17 | MFGE8 | Lactadherin | 2.848 | 1.807 | 2.778 |
| 18 | VIM | Vimentin | 2.806 | 2.951 | 2.838 |
| 19 | HMGN3 | High mobility group | 2.780 | 3.481 | 2.733 |
| | | nucleosome-binding | | | |
| | | domain-containing | | | |
| | | protein 3 | | | |
| 20 | CCDC50 | Isoform 2 of Coiled- | 2.766 | 2.846 | 2.665 |
| | | coil domain- | | | |
| | | containing protein | | | |
| | | 50 | | | |
| 21 | GAP43 | Isoform 2 of | 2.743 | 3.020 | 2.812 |
| | | Neuromodulin | | | |
| 22 | STMN3 | Stathmin-3 | 2.721 | 3.127 | 2.883 |
| 23 | NOP53 | Ribosome biogenesis | 2.718 | 3.232 | 2.757 |
| | | protein NOP53 | | | |
| 24 | ALYREF | THO complex | 2.716 | 3.348 | 2.725 |
| | | subunit 4 | | | |
| 25 | SEPTIN9 | Septin-9 | 2.659 | 2.990 | 2.562 |
| 26 | REEP5 | Receptor expression- | 2.645 | 2.409 | 2.698 |
| | | enhancing protein 5 | | | |
| 27 | ALDOC | Fructose- | 2.629 | 1.276 | 2.645 |
| | | bisphosphate | | | |
| | | aldolase C | | | |
| 28 | BCAP31 | Isoform 2 of B-cell | 2.617 | 2.174 | 2.671 |
| | | receptor-associated | | | |
| | | protein 31 | | | |
| 29 | CRISPLD1 | Cysteine-rich | 2.611 | 1.673 | 2.387 |
| | | secretory protein | | | |
| | | LCCL domain- | | | |
| | | containing 1 | | | |
| 30 | DKK1 | Dickkopf-related | 2.586 | 1.063 | 2.617 |
| | | protein 1 | | | |
| 31 | CHTOP | Isoform 2 of | 2.585 | 2.490 | 2.360 |
| | | Chromatin target of | | | |
| | | PRMT1 protein | | | |
| 32 | CCDC137 | Coiled-coil domain- | 2.544 | 2.857 | 2.402 |
| | | containing protein | | | |
| | | 137 | | | |
| 33 | SPRY4 | Protein sprouty | 2.518 | 1.239 | 2.437 |
| | | homolog 4 | | | |
| 34 | EIF4H | Eukaryotic | 2.463 | 2.673 | 2.448 |
| | | translation initiation | | | |
| | | factor 4H | | | |
| 35 | ACIN1 | Apoptotic chromatin | 2.460 | 2.824 | 2.267 |
| | | condensation inducer | | | |
| | | in the nucleus | | | |
| 36 | CHGA | Chromogranin-A | 2.457 | 2.085 | 2.799 |
| 37 | PEBP1 | Phosphatidylethanol | 2.430 | 2.305 | 2.515 |
| | | amine-binding | | | |
| | | protein 1 | | | |
| 38 | HMGN1 | Non-histone | 2.425 | 3.156 | 2.459 |
| | | chromosomal protein | | | |
| | | HMG-14 | | | |
| 39 | PLIN3 | Perilipin-3 | 2.423 | 2.303 | 2.423 |
| 40 | STMN2 | Isoform 2 of | 2.421 | 2.286 | 2.527 |
| | | Stathmin-2 | | | |
| 41 | TPI1 | Isoform 2 of | 2.415 | 2.189 | 2.410 |
| | | Triosephosphate | | | |
| | | isomerase | | | |
| 42 | KIDINS220 | Kinase D-interacting | 2.377 | 1.407 | 2.306 |
| | | substrate of 220 kDa | | | |
| 43 | RBMXL1 | RNA binding motif | 2.366 | 2.596 | 2.497 |
| | | protein, X-linked- | | | |
| | | like-1 | | | |
| 44 | CTTN | Src substrate | 2.365 | 2.375 | 2.321 |
| | | cortactin | | | |
| 45 | LDHA | Isoform 3 of L- | 2.352 | 2.297 | 2.366 |
| | | lactate | | | |
| | | dehydrogenase A | | | |
| | | chain | | | |
| 46 | GDI2 | Rab GDP | 2.333 | 1.966 | 2.435 |
| | | dissociation inhibitor | | | |
| | | beta | | | |
| 47 | CYCS | Cytochrome c | 2.316 | 2.659 | 2.251 |
| 48 | YLPM1 | YLP motif- | 2.310 | 2.769 | 2.305 |
| | | containing protein 1 | | | |
| 49 | NOLC1 | Isoform Beta of | 2.304 | 2.698 | 2.160 |
| | | Nucleolar and | | | |
| | | coiled-body | | | |
| | | phosphoprotein 1 | | | |
| 50 | GDI1 | Rab GDP | 2.295 | 1.765 | 2.381 |
| | | dissociation inhibitor | | | |
| | | alpha | | | |

Furthermore, to present the normalized counts of the upregulated proteins in each group (Gigasome Group A, B, C, or Apoptotic bodies), the following formula was used:

$$Normalized count = Average ( Raw count values of Protein X ) /$$

$$Average ( Raw count values of 5 housekeeping proteins ) * 100$$

The replicates of the raw counts for each protein in each group were averaged. The averaged raw count values for each specific protein were then normalized to the average raw counts of the five housekeeping proteins within each group (e.g., the raw counts of protein Galectin-1 (LGALS1) in triplicate in Group A were first averaged, and then normalized to the average of the five housekeeping proteins (FIG. 12) in the Group A.) Table 17 shows the normalized counts of the top 50 significantly upregulated proteins.

TABLE 17
Counts of significantly upregulated proteins across all three Gigasomes Groups and the Apoptotic
bodies normalized to the housekeeping proteins within that group. Proteins are sorted by
order of log2 fold change. Only proteins with p-value < 0.05 are included in table.

| | | | Gigasome | Gigasome | Gigasome | Apoptotic |
|---|---|---|---|---|---|---|
| | | | Group A | Group B | Group C | bodies |
| | | | (Normalized | (Normalized | (Normalized | (Normalized |
| No. | Gene ID | Protein name | counts) | counts) | counts) | counts) |
| 1 | PANX1 | Pannexin-1 | 41.26 | 47.86 | 35.66 | 7.01 |
| 2 | PDAP1 | 28 kDa heat- and acid- | 53.05 | 34.05 | 52.16 | 6.07 |
| | | stable phosphoprotein | | | | |
| 3 | CIRBP | Cold-inducible RNA- | 30.03 | 10.80 | 31.09 | 4.68 |
| | | binding protein | | | | |
| 4 | RBMX | RNA-binding motif | 37.22 | 53.57 | 37.60 | 5.55 |
| | | protein, X | | | | |
| | | chromosome | | | | |
| 5 | PTMA | Prothymosin alpha | 143.76 | 95.58 | 148.82 | 21.80 |
| 6 | BCL7A | Isoform 2 of B-cell | 25.69 | 34.92 | 23.75 | 2.67 |
| | | CLL/lymphoma 7 | | | | |
| | | protein family | | | | |
| | | member A | | | | |
| 7 | STMN1 | Isoform 2 of Stathmin | 12.01 | 13.19 | 10.50 | 1.86 |
| 8 | PLTP | Phospholipid transfer | 5.73 | 5.42 | 5.49 | 0.81 |
| | | protein | | | | |
| 9 | HMGN2 | Non-histone | 11.11 | 14.32 | 10.50 | 1.38 |
| | | chromosomal protein | | | | |
| | | HMG-17 | | | | |
| 10 | PYM1 | Partner of Y14 and | 18.32 | 12.48 | 22.44 | 3.19 |
| | | mago | | | | |
| 11 | AKAP12 | A-kinase anchor | 6.94 | 6.54 | 5.67 | 1.22 |
| | | protein 12 | | | | |
| 12 | CHCHD2 | Coiled-coil-helix- | 6.71 | 9.26 | 6.51 | 0.50 |
| | | coiled-coil-helix | | | | |
| | | domain-containing | | | | |
| | | protein 2 | | | | |
| 13 | MDK | Midkine | 15.73 | 7.61 | 13.83 | 2.48 |
| 14 | TMSB10 | Thymosin beta-10 | 134.43 | 121.05 | 129.99 | 24.30 |
| 15 | TMA7 | Translation machinery- | 57.34 | 68.54 | 56.31 | 11.51 |
| | | associated protein 7 | | | | |
| 16 | MRPL34 | 39S ribosomal protein | 31.50 | 10.43 | 32.53 | 5.04 |
| | | L34, mitochondrial | | | | |
| 17 | MFGE8 | Lactadherin | 38.68 | 41.00 | 38.72 | 6.68 |
| 18 | VIM | Vimentin | 125.09 | 136.86 | 131.05 | 17.27 |
| 19 | HMGN3 | High mobility group | 302.44 | 190.27 | 327.63 | 58.21 |
| | | nucleosome-binding | | | | |
| | | domain-containing | | | | |
| | | protein 3 | | | | |
| 20 | CCDC50 | Isoform 2 of Coiled- | 138.74 | 96.56 | 149.98 | 26.19 |
| | | coil domain-containing | | | | |
| | | protein 50 | | | | |
| 21 | GAP43 | Isoform 2 of | 35.63 | 53.59 | 36.03 | 6.17 |
| | | Neuromodulin | | | | |
| 22 | STMN3 | Stathmin-3 | 12.64 | 19.50 | 13.02 | 1.40 |
| 23 | NOP53 | Ribosome biogenesis | 6.39 | 9.66 | 6.15 | 0.87 |
| | | protein NOP53 | | | | |
| 24 | ALYREF | THO complex subunit 4 | 25.05 | 11.46 | 23.56 | 4.44 |
| 25 | SEPTIN9 | Septin-9 | 203.62 | 191.65 | 207.68 | 38.01 |
| 26 | REEP5 | Receptor expression- | 90.75 | 58.57 | 82.31 | 11.23 |
| | | enhancing protein 5 | | | | |
| 27 | ALDOC | Fructose-bisphosphate | 97.74 | 42.65 | 93.59 | 12.87 |
| | | aldolase C | | | | |
| 28 | BCAP31 | Isoform 2 of B-cell | 15.56 | 15.79 | 16.54 | 2.03 |
| | | receptor-associated | | | | |
| | | protein 31 | | | | |
| 29 | CRISPLD1 | Cysteine-rich secretory | 78.36 | 87.91 | 65.14 | 13.91 |
| | | protein LCCL domain- | | | | |
| | | containing 1 | | | | |
| 30 | DKK1 | Dickkopf-related | 11.74 | 14.82 | 11.56 | 1.69 |
| | | protein 1 | | | | |
| 31 | CHTOP | Isoform 2 of | 7.23 | 2.33 | 7.22 | 0.40 |
| | | Chromatin target of | | | | |
| | | PRMT1 protein | | | | |
| 32 | CCDC137 | Coiled-coil domain- | 122.11 | 110.05 | 115.03 | 7.94 |
| | | containing protein 137 | | | | |
| 33 | SPRY4 | Protein sprouty | 172.14 | 144.66 | 184.60 | 30.28 |
| | | homolog 4 | | | | |
| 34 | EIF4H | Eukaryotic translation | 54.77 | 45.94 | 55.25 | 9.50 |
| | | initiation factor 4H | | | | |
| 35 | ACIN1 | Apoptotic chromatin | 4.98 | 6.87 | 6.36 | 0.53 |
| | | condensation inducer | | | | |
| | | in the nucleus | | | | |
| 36 | CHGA | Chromogranin-A | 27.90 | 61.75 | 41.36 | 2.66 |
| 37 | PEBP1 | Phosphatidylethanola | 57.46 | 54.11 | 57.94 | 6.38 |
| | | mine-binding protein 1 | | | | |
| 38 | HMGN1 | Non-histone | 83.08 | 81.50 | 73.13 | 5.26 |
| | | chromosomal protein | | | | |
| | | HMG-14 | | | | |
| 39 | PLIN3 | Perilipin-3 | 4.32 | 4.60 | 4.68 | 0.78 |
| 40 | STMN2 | Isoform 2 of Stathmin-2 | 36.14 | 27.61 | 37.14 | 5.34 |
| 41 | TPI1 | Isoform 2 of | 3.50 | 4.12 | 3.37 | 0.53 |
| | | Triosephosphate | | | | |
| | | isomerase | | | | |
| 42 | KIDINS220 | Kinase D-interacting | 14.74 | 5.50 | 13.84 | 2.42 |
| | | substrate of 220 kDa | | | | |
| 43 | RBMXL1 | RNA binding motif | 177.17 | 160.99 | 179.25 | 17.71 |
| | | protein, X-linked-like-1 | | | | |
| 44 | CTTN | Src substrate cortactin | 62.38 | 50.95 | 66.49 | 10.84 |
| 45 | LDHA | Isoform 3 of L-lactate | 9.26 | 11.42 | 10.82 | 1.39 |
| | | dehydrogenase A | | | | |
| | | chain | | | | |
| 46 | GDI2 | Rab GDP dissociation | 16.91 | 27.19 | 17.00 | 2.34 |
| | | inhibitor beta | | | | |
| 47 | CYCS | Cytochrome c | 19.70 | 16.19 | 19.98 | 2.40 |
| 48 | YLPM1 | YLP motif-containing | 322.26 | 258.35 | 330.01 | 58.03 |
| | | protein 1 | | | | |
| 49 | NOLC1 | Isoform Beta of | 425.95 | 438.96 | 455.36 | 58.75 |
| | | Nucleolar and coiled- | | | | |
| | | body phosphoprotein 1 | | | | |
| 50 | GDI1 | Rab GDP dissociation | 8.89 | 11.28 | 8.85 | 1.67 |
| | | inhibitor alpha | | | | |

Similarly, to identify downregulated proteins in the gigasomes as compared to the apoptotic bodies, first the raw values of all the proteins were transformed using log 2. Next, the log 2 transformed values of every protein was normalized to the average log 2 values of five housekeeping proteins identified in FIG. 12. This normalization was performed first, by computing the average of the five housekeeping proteins across each group. Next, the deviation from the average for each group was calculated by subtracting the average across all groups from the average of each group. The resulting deviation per group were then subtracted from the log 2 values of each protein in that group. These log 2 values were then averaged across replicates per group. Next, in order to calculate fold changes of each protein in the gigasome groups compared to the apoptotic bodies, fold changes were calculated by using the formula:

$$Log 2 Fold Change ( Log 2 FC ) = {X}_{A} - {X}_{AB}$$

Where X_{A}=Averaged normalized log 2 transformed counts of a specific protein from a gigasome group
X_{AB}=Averaged normalized log 2 transformed counts of same protein from the apoptotic bodies group

Next, to find significantly different proteins, a p-value cut-off was established. Significances denoted by p-values were calculated using a two-tailed Student's T-test comparing two sample unequal variances (type 3) for X_{A}, X_{AB}, and a p-value of <0.05 was considered significant. Only proteins that were significantly downregulated in all the 3 gigasome groups as compared to the apoptotic bodies were considered for further analysis. Then, the resulting list of proteins were sorted based on the lowest log 2 fold change in Gigasome group A in comparison to the apoptotic bodies group, followed by gigasome Group B vs apoptotic bodies and finally Gigasome C vs apoptotic bodies. Finally, only proteins that were present in all the three gigasome groups were considered. This analysis yielded 230 significantly downregulated proteins in the gigasome groups A, B and C as compared to the apoptotic bodies. Table 18 presents the top 50 proteins with the lowest Log 2 fold change values after the p-value cut-off of <0.05 in the Gigasome groups A, B and C compared to Apoptotic bodies.

TABLE 18
Log2 fold change values of significantly downregulated proteins across all three
Gigasomes Groups compared to Apoptotic bodies. Proteins are sorted by order of
log2 fold change. Only proteins with p-value < 0.05 are included in table.

| | | | Gigasome | Gigasome | Gigasome |
|---|---|---|---|---|---|
| | | | Group A | Group B | Group C |
| | | | Log2 Fold Change | Log2 Fold Change | Log2 Fold Change |
| | | | from Apoptotic | from Apoptotic | from Apoptotic |
| No. | Gene ID | Protein name | bodies | bodies | bodies |
| 1 | RPA3 | Replication protein A 14 | −2.161 | −1.376 | −2.137 |
| | | kDa subunit | | | |
| 2 | POLD1 | DNA polymerase delta | −2.121 | −1.352 | −1.801 |
| | | catalytic subunit | | | |
| 3 | CARHSP1 | Calcium-regulated heat- | −2.074 | −1.330 | −1.742 |
| | | stable protein 1 | | | |
| 4 | RPS12 | 40S ribosomal protein S12 | −1.995 | −1.725 | −1.578 |
| 5 | LGALS1 | Galectin-1 | −1.879 | −1.642 | −1.682 |
| 6 | MCM4 | DNA replication licensing | −1.869 | −0.869 | −1.705 |
| | | factor MCM4 | | | |
| 7 | COL2A1 | Collagen alpha-1(II) chain | −1.856 | −2.199 | −1.647 |
| 8 | NDUFA5 | NADH dehydrogenase | −1.847 | −1.557 | −1.622 |
| | | [ubiquinone] 1 alpha | | | |
| | | subcomplex subunit 5 | | | |
| 9 | ANLN | Anillin OS = Homo sapiens | −1.841 | −0.791 | −1.636 |
| 10 | DHFR | Dihydrofolate reductase | −1.820 | −0.989 | −1.620 |
| 11 | DDX5 | Probable ATP-dependent | −1.817 | −0.906 | −1.681 |
| | | RNA helicase DDX5 | | | |
| 12 | MCM6 | DNA replication licensing | −1.812 | −0.881 | −1.688 |
| | | factor MCM6 | | | |
| 13 | ILKAP | Integrin-linked kinase- | −1.797 | −0.776 | −1.638 |
| | | associated serine/threonine | | | |
| | | phosphatase 2C | | | |
| 14 | LIG1 | DNA ligase 1 | −1.790 | −0.849 | −1.690 |
| 15 | HNRNPM | Heterogeneous nuclear | −1.768 | −1.193 | −1.689 |
| | | ribonucleoprotein M | | | |
| 16 | MCM5 | DNA replication licensing | −1.760 | −0.987 | −1.603 |
| | | factor MCM5 | | | |
| 17 | MCM2 | DNA replication licensing | −1.742 | −0.764 | −1.622 |
| | | factor MCM2 | | | |
| 18 | MSH2 | DNA mismatch repair | −1.730 | −0.883 | −1.575 |
| | | protein Msh2 | | | |
| 19 | FANCI | Fanconi anemia group I | −1.712 | −0.624 | −1.376 |
| | | protein | | | |
| 20 | TPRKB | Isoform 3 of EKC/KEOPS | −1.711 | −1.329 | −1.970 |
| | | complex subunit TPRKB | | | |
| 21 | HNRNPL | Heterogeneous nuclear | −1.674 | −0.967 | −1.521 |
| | | ribonucleoprotein L | | | |
| 22 | FAM98B | Protein FAM98B | −1.654 | −1.124 | −1.527 |
| 23 | MCM7 | DNA replication licensing | −1.650 | −0.909 | −1.540 |
| | | factor MCM7 | | | |
| 24 | EEF1A1 | Elongation factor 1-alpha 1 | −1.640 | −1.171 | −1.532 |
| 25 | PTGES3 | Prostaglandin E synthase 3 | −1.625 | −1.332 | −1.526 |
| 26 | LYPLA1 | Acyl-protein thioesterase 1 | −1.623 | −1.445 | −1.380 |
| 27 | TUBB4B | Tubulin beta-4B chain | −1.623 | −1.262 | −1.549 |
| 28 | NTMT1 | N-terminal Xaa-Pro-Lys N- | −1.617 | −2.106 | −1.612 |
| | | methyltransferase 1 | | | |
| 29 | ANP32E | Acidic leucine-rich nuclear | −1.578 | −0.672 | −1.426 |
| | | phosphoprotein 32 family | | | |
| | | member E | | | |
| 30 | HNRNPM | Heterogeneous nuclear | −1.578 | −0.478 | −1.321 |
| | | ribonucleoprotein M | | | |
| | | (Fragment) | | | |
| 31 | SMC2 | Structural maintenance of | −1.575 | −0.978 | −1.558 |
| | | chromosomes protein 2 | | | |
| 32 | BOLA2 | BolA-like protein 2 | −1.562 | −1.420 | −1.215 |
| 33 | ACP1 | Low molecular weight | −1.554 | −0.704 | −1.242 |
| | | phosphotyrosine protein | | | |
| | | phosphatase | | | |
| 34 | PAPSS1 | Bifunctional 3′- | −1.546 | −1.261 | −1.618 |
| | | phosphoadenosine 5′- | | | |
| | | phosphosulfate synthase 1 | | | |
| 35 | PBK | Isoform 2 of Lymphokine- | −1.522 | −0.615 | −1.463 |
| | | activated killer T-cell- | | | |
| | | originated protein kinase | | | |
| 36 | PNKP | Bifunctional polynucleotide | −1.517 | −1.421 | −1.491 |
| | | phosphatase/kinase | | | |
| 37 | ELOC | Elongin-C | −1.511 | −0.984 | −1.281 |
| 38 | VRK1 | Serine/threonine-protein | −1.498 | −0.908 | −1.358 |
| | | kinase VRK1 | | | |
| 39 | MSH6 | DNA mismatch repair | −1.492 | −0.883 | −1.407 |
| | | protein Msh6 | | | |
| 40 | FRG1 | Protein FRG1 | −1.489 | −0.525 | −1.347 |
| 41 | CTBP2 | Isoform 2 of C-terminal- | −1.480 | −0.935 | −1.435 |
| | | binding protein 2 | | | |
| 42 | MCM3 | Isoform 2 of DNA | −1.476 | −0.825 | −1.422 |
| | | replication licensing factor | | | |
| | | MCM3 | | | |
| 43 | IRF2BPL | Probable E3 ubiquitin- | −1.474 | −0.794 | −1.404 |
| | | protein ligase IRF2BPL | | | |
| 44 | TAF15 | TATA-binding protein- | −1.468 | −0.825 | −1.396 |
| | | associated factor 2N | | | |
| 45 | ITPA | Inosine triphosphate | −1.463 | −1.012 | −1.014 |
| | | pyrophosphatase | | | |
| 46 | WDR70 | WD repeat-containing | −1.447 | −0.814 | −1.363 |
| | | protein 70 | | | |
| 47 | KIN | DNA/RNA-binding protein | −1.446 | −0.765 | −1.362 |
| | | KIN17 | | | |
| 48 | SRSF9 | Serine/arginine-rich | −1.432 | −0.836 | −1.521 |
| | | splicing factor 9 | | | |
| 49 | RNGTT | mRNA-capping enzyme | −1.432 | −0.651 | −1.356 |
| 50 | FKBP5 | Peptidyl-prolyl cis-trans | −1.428 | −0.836 | −1.293 |
| | | isomerase FKBP5 | | | |

Furthermore, to present the normalized counts of the downregulated proteins in each group (Gigasome Group A, B, C, or Apoptotic bodies), the following formula was used:

$$Normalized count = Average ( Raw count values of Protein X ) /$$

$$Average ( Raw count values of 5 housekeeping proteins ) * 100$$

The replicates of the raw counts for each protein in each group were averaged. The averaged raw count values for each specific protein were then normalized to the average raw counts of the five housekeeping proteins within each group (e.g., the raw counts of protein Galectin-1 (LGALS1) in triplicate in Group A were first averaged, and then normalized to the average of the five housekeeping proteins (FIG. 12) in the Group A.) Table 19 shows the normalized counts of the top 50 significantly downregulated proteins.

TABLE 19
Counts of significantly downregulated proteins across all three Gigasomes Groups and the Apoptotic
bodies normalized to the housekeeping proteins within that group. Proteins are sorted by
order of log2 fold change. Only proteins with p-value < 0.05 are included in table.

| | | | Gigasome | Gigasome | Gigasome | Apoptotic |
|---|---|---|---|---|---|---|
| | | | Group A | Group B | Group C | bodies |
| | | | (Normalized | (Normalized | (Normalized | (Normalized |
| No. | Gene ID | Protein name | counts) | counts) | counts) | counts) |
| 1 | RPA3 | Replication protein A 14 | 19.10 | 30.71 | 23.07 | 56.29 |
| | | kDa subunit | | | | |
| 2 | POLD1 | DNA polymerase delta | 8.39 | 15.94 | 9.53 | 30.22 |
| | | catalytic subunit | | | | |
| 3 | CARHSP1 | Calcium-regulated heat- | 22.19 | 37.82 | 23.94 | 66.07 |
| | | stable protein 1 | | | | |
| 4 | RPS12 | 40S ribosomal protein S12 | 10.12 | 9.97 | 12.56 | 30.05 |
| 5 | LGALS1 | Galectin-1 | 7.08 | 10.62 | 8.65 | 30.01 |
| 6 | MCM4 | DNA replication licensing | 7.08 | 5.01 | 7.79 | 26.08 |
| | | factor MCM4 | | | | |
| 7 | COL2A1 | Collagen alpha-1(II) chain | 29.37 | 39.46 | 29.33 | 82.11 |
| 8 | NDUFA5 | NADH dehydrogenase | 116.51 | 197.77 | 125.58 | 418.41 |
| | | [ubiquinone] 1 alpha | | | | |
| | | subcomplex subunit 5 | | | | |
| 9 | ANLN | Anillin OS = Homo sapiens | 25.97 | 41.86 | 29.13 | 93.31 |
| 10 | DHFR | Dihydrofolate reductase | 88.00 | 108.51 | 91.87 | 275.92 |
| 11 | DDX5 | Probable ATP-dependent | 30.24 | 37.99 | 33.53 | 83.73 |
| | | RNA helicase DDX5 | | | | |
| 12 | MCM6 | DNA replication licensing | 19.55 | 25.35 | 20.93 | 62.69 |
| | | factor MCM6 | | | | |
| 13 | ILKAP | Integrin-linked kinase- | 6.67 | 12.72 | 8.23 | 21.76 |
| | | associated serine/threonine | | | | |
| | | phosphatase 2C | | | | |
| 14 | LIG1 | DNA ligase 1 | 27.83 | 37.45 | 29.68 | 74.68 |
| 15 | HNRNPM | Heterogeneous nuclear | 19.32 | 33.96 | 20.76 | 54.04 |
| | | ribonucleoprotein M | | | | |
| 16 | MCM5 | DNA replication licensing | 30.67 | 44.80 | 33.26 | 98.33 |
| | | factor MCM5 | | | | |
| 17 | MCM2 | DNA replication licensing | 71.60 | 97.32 | 73.81 | 251.12 |
| | | factor MCM2 | | | | |
| 18 | MSH2 | DNA mismatch repair | 1.91 | 3.61 | 2.18 | 5.62 |
| | | protein Msh2 | | | | |
| 19 | FANCI | Fanconi anemia group I | 1.84 | 3.38 | 2.00 | 6.43 |
| | | protein | | | | |
| 20 | TPRKB | Isoform 3 of EKC/KEOPS | 6.28 | 9.02 | 6.41 | 17.53 |
| | | complex subunit TPRKB | | | | |
| 21 | HNRNPL | Heterogeneous nuclear | 27.50 | 32.89 | 35.55 | 73.83 |
| | | ribonucleoprotein L | | | | |
| 22 | FAM98B | Protein FAM98B | 10.19 | 14.92 | 10.47 | 27.73 |
| 23 | MCM7 | DNA replication licensing | 127.04 | 131.87 | 137.79 | 469.39 |
| | | factor MCM7 | | | | |
| 24 | EEF1A1 | Elongation factor 1-alpha 1 | 17.14 | 29.52 | 17.88 | 59.64 |
| 25 | PTGES3 | Prostaglandin E synthase 3 | 17.86 | 17.78 | 20.14 | 53.92 |
| 26 | LYPLA1 | Acyl-protein thioesterase 1 | 97.20 | 171.78 | 103.22 | 324.97 |
| 27 | TUBB4B | Tubulin beta-4B chain | 122.21 | 176.14 | 123.39 | 341.22 |
| 28 | NTMT1 | N-terminal Xaa-Pro-Lys | 58.48 | 104.85 | 63.95 | 214.07 |
| | | N-methyltransferase 1 | | | | |
| 29 | ANP32E | Acidic leucine-rich nuclear | 84.13 | 130.17 | 91.96 | 284.64 |
| | | phosphoprotein 32 family | | | | |
| | | member E | | | | |
| 30 | HNRNPM | Heterogeneous nuclear | 74.81 | 128.47 | 80.29 | 263.94 |
| | | ribonucleoprotein M | | | | |
| | | (Fragment) | | | | |
| 31 | SMC2 | Structural maintenance of | 106.46 | 161.97 | 112.63 | 335.69 |
| | | chromosomes protein 2 | | | | |
| 32 | BOLA2 | BolA-like protein 2 | 8.51 | 14.04 | 9.25 | 28.18 |
| 33 | ACP1 | Low molecular weight | 54.39 | 75.78 | 56.14 | 153.50 |
| | | phosphotyrosine protein | | | | |
| | | phosphatase | | | | |
| 34 | PAPSS1 | Bifunctional 3′- | 14.82 | 16.35 | 16.82 | 55.06 |
| | | phosphoadenosine 5′- | | | | |
| | | phosphosulfate synthase 1 | | | | |
| 35 | PBK | Isoform 2 of Lymphokine- | 2.89 | 2.12 | 2.83 | 8.95 |
| | | activated killer T-cell- | | | | |
| | | originated protein kinase | | | | |
| 36 | PNKP | Bifunctional | 41.44 | 45.49 | 38.38 | 124.16 |
| | | polynucleotide | | | | |
| | | phosphatase/kinase | | | | |
| 37 | ELOC | Elongin-C | 10.14 | 17.20 | 10.33 | 29.16 |
| 38 | VRK1 | Serine/threonine-protein | 6.51 | 6.60 | 6.45 | 19.08 |
| | | kinase VRK1 | | | | |
| 39 | MSH6 | DNA mismatch repair | 1.88 | 2.89 | 2.30 | 8.16 |
| | | protein Msh6 | | | | |
| 40 | FRG1 | Protein FRG1 | 78.31 | 89.16 | 81.19 | 250.62 |
| 41 | CTBP2 | Isoform 2 of C-terminal- | 22.97 | 35.68 | 23.68 | 62.09 |
| | | binding protein 2 | | | | |
| 42 | MCM3 | Isoform 2 of DNA | 1.28 | 1.94 | 1.25 | 5.65 |
| | | replication licensing factor | | | | |
| | | MCM3 | | | | |
| 43 | IRF2BPL | Probable E3 ubiquitin- | 27.55 | 29.84 | 35.59 | 109.66 |
| | | protein ligase IRF2BPL | | | | |
| 44 | TAF15 | TATA-binding protein- | 139.57 | 190.64 | 137.17 | 415.22 |
| | | associated factor 2N | | | | |
| 45 | ITPA | Inosine triphosphate | 33.34 | 45.35 | 30.31 | 90.14 |
| | | pyrophosphatase | | | | |
| 46 | WDR70 | WD repeat-containing | 23.53 | 33.50 | 23.79 | 65.06 |
| | | protein 70 | | | | |
| 47 | KIN | DNA/RNA-binding | 9.58 | 11.53 | 7.69 | 31.31 |
| | | protein KIN17 | | | | |
| 48 | SRSF9 | Serine/arginine-rich | 17.06 | 19.61 | 17.61 | 52.74 |
| | | splicing factor 9 | | | | |
| 49 | RNGTT | mRNA-capping enzyme | 22.47 | 30.78 | 24.16 | 63.49 |
| 50 | FKBP5 | Peptidyl-prolyl cis-trans | 6.61 | 9.16 | 6.93 | 18.18 |
| | | isomerase FKBP5 | | | | |

### Example 17: Small Molecule Compound Screen to Reveal Up-Regulators of Gigasome Production in Neuronal Monocultures

This example demonstrates that gigasome production can be differentially up-regulated from the baseline in a neuronal cell line derived from human neuroblastoma upon treatment with various compounds targeting different pathways, including Proteasome, Autophagy, Membrane Trafficking Proliferation and Longevity, Inflammation and Receptor Target, Disease Relevant and Metabolism and Stress Signaling pathways.

To quantify and characterize gigasome production using microscopy, while tracking cell viability/phenotype, a multi-readout system was developed that allowed simultaneous analysis of gigasomes harvested from cell culture media supernatants and respective gigasome producing cell viability. Cells were stained with Hoechst 34580, CellTracker Green/CellMask Green Actin and MitoTracker Deep Red to detect nuclear material, cytoplasm/F-actin, and mitochondria, respectively, and cultured in 96-well glass bottom plates at 42,000 cells/cm² and allowed to adhere overnight. For the screening, a total of 99 compounds (TargetMol) were divided in 5 groups. Each group included an untreated condition, a vehicular DMSO control, and a known high-gigasome producing condition to comparatively study gigasome production rates. As a cell death control group, 500 nM Staurosporine was added to induce apoptosis. Cultures were incubated for 24 hours under gigasome inducing conditions at concentrations ranging between 10 nM and 10,000 nM. Before the end of the treatment, live neuronal cultures were imaged. The Zeiss ZEN Blue software was used to distribute imaging fields of view (positions) to create an unbiased imaging sample of a well. 5 images were taken using a Zeiss LSM 900 confocal microscope. Images were in 16-bit format, and contained at least an ESID (brightfield) channel, a Hoechst 34580 fluorescence channel, a CellTracker Green/Cell Mask Green Actin fluorescence channel, and a MitoTracker Deep Red fluorescence channel. The BioApps ZEN software package was used to perform cell nucleus segmentation and counting. After 24 hours, gigasomes were harvested from the cell culture media by gently removing from the top of each 96-well ⅔ of the total volume without disturbing the cell layer (containing the settled gigasomes) and by adding to each well the same amount of PBS. The wells were washed twice gently, and the gigasome-containing media/PBS wash was transferred into the well of a 384-well glass bottom plate. Cells remaining in the 96-well plates were assessed for viability using CellTiter-Glo (Promega).

For gigasome analysis in 384-well plates, the Zeiss ZEN Blue software was used to distribute positions to create an unbiased imaging sample of a well. 6 images per well were taken using a Zeiss LSM 900 confocal microscope. Images were in 16-bit format, and contained at least an ESID (brightfield) channel, a Hoechst 34580 fluorescence channel, a CellTracker Green/Cell Mask Green Actin fluorescence channel, and a MitoTracker Deep Red fluorescence channel.

The CellTracker Green/Cell Mask Green fluorescence channel threshold was set between 5403 and 65535 for 16-bit images. A watershed binary segmentation step was performed to separate grouped objects. For each particle in the mask, characteristic parameters of the particle were calculated and saved.

In some experiments, these parameters were the particles area, circularity, mean fluorescence signal in the Hoechst 34580 channel (nuclear signal), and mean fluorescence signal in the MitoTracker Deep Red channel (mitochondrial signal). Particles of interest were identified by thresholding by area and circularity. Area was restricted to be between 7.07 and 314 μm² (which correspond to spheres with diameters ranging from 3 to 20 μm). Circularity, defined by the formula 4pi(area/perimeter\hat{ }2, was set to be between 0.8 and 1, where a value of 1.0 is a perfect circle, and a value of 0 is a line. Gigasomes were identified from the particles of interest by thresholding by mean nuclear signal. The nuclear signal threshold was set using a value equal to one fourth of a threshold value calculated by the Li auto-threshold algorithm applied to the distribution of all particles of interest. Particles of interest with nuclear signal below this value were considered “negative” for nuclear material and were identified as gigasomes. A sampling factor was calculated to extrapolate the total quantity of a type of object (particles, particles of interest, gigasomes) in the well. The sampling factor was the percentage of the well's area that was imaged. Dividing a quantity or distribution of objects by the sampling factor resulted in an estimated quantity or distribution of objects for the whole well. In some experiments, the number of cells in the cell cultures were estimated with a cell counter. Dividing a quantity or distribution of objects by the number of cells from the culture that produced them resulted in a normalized quantity or distribution. The normalized quantity or distribution of objects was described as a quantity or distribution of objects per 1000 cells.

Neuroblastoma cells treated with compounds targeting different pathways displayed differential rates of gigasome production. By setting cell viability at ≥75% and gigasome counts ≥1.5 fold compared to the vehicular DMSO control, gigasome producers were identified in at least 11% and up to 57% of compound categories tested which are involved in membrane trafficking, proteasome; autophagy; inflammation and receptor target; metabolism and stress signaling; or proliferation and longevity.

The top compounds identified increasing gigasome production 2-fold or more compared to DMSO (Table 20, bold text) played a role in inhibition of endosomal trafficking (10,000 nM MiTMAB), inhibition of proteasome function (1000 nM, Tripterin), inhibition of late-stage autophagy (100-10,000 nM Bafilomycin A1), induction of autophagy via MEK signaling inhibition (1000 nM Trametinib), and inhibition of glutathione peroxidase activity (1000 nM RSL3).

TABLE 20
Small molecule compounds that demonstrated ability to modulate gigasome production in neuronal cells.
Each compound is labelled with its targeted pathway, known mechanism of action of the compound, dose
used in the screen, number of gigasomes produced, fold change of the number of gigasomes produced
compared to the DMSO control, and the percentage viability. Compounds identified increasing gigasome
production 2-fold or more compared to DMSO are bolded. At times, multiples doses are highlighted.

| | | | | Number of | Fold Change | |
|---|---|---|---|---|---|---|
| | | Compound | Dose | Gigasomes | of Gigasomes | Viability |
| Pathway | Mechanism of Action | Name | (nM) | Produced | Produced vs. DMSO | (%) |
| N/A | N/A | DMSO | N/A | <35 | 1.0 | 100 |
| Membrane | Endocytosis Inhibitor | MiTMAB | 10000 | 55-65 | 2.0-3.0 | >75 |
| Trafficking | ER to Golgi Inhibitor | Brefeldin A | 10 | 45-55 | 1.5-1.75 | 100 |
| | Exocytosis Inhibitor | Tipifarnib | 1000 | 65-75 | 1.75-2.0 | 50-75 |
| | Exocytosis Inhibitor | Tipifarnib | 100 | 55-65 | 1.5-1.75 | >75 |
| | Exocytosis Inhibitor | Tipifarnib | 10 | <35 | 0.75-1.0 | >75 |
| | ER to Golgi Inhibitor | Monensin | 10000 | 55-65 | 1.5-1.75 | 50-75 |
| | | sodium salt | | | | |
| | ER to Golgi Inhibitor | Monensin | 100 | 55-65 | 1.75-2.0 | >75 |
| | | sodium salt | | | | |
| | ER to Golgi Inhibitor | Monensin | 10 | <35 | 0.75-1.0 | >75 |
| | | sodium salt | | | | |
| | Exocytosis Inhibitor | Simvastatin | 10000 | 45-55 | 1.25-1.5 | >75 |
| | Exocytosis Inhibitor | Simvastatin | 1000 | 35-45 | 1.0-1.25 | 100 |
| | Exocytosis Inhibitor | Simvastatin | 100 | <35 | 0.75-1.0 | 100 |
| Proteasome | Inhibitor | Tripterin | 1000 | 75-85 | 2.0-3.0 | >75 |
| | Inhibitor | MG-132 | 100 | 55-65 | 1.5-1.75 | 50-75 |
| | Activator | Betulinic acid | 10000 | 55-65 | 1.5-1.75 | 100 |
| | Activator | Betulinic acid | 100 | 55-65 | 1.25-1.5 | 100 |
| | Activator | Betulinic acid | 10 | <35 | 0.5-0.75 | 100 |
| Autophagy | Inhibitor | Bafilomycin | 10000 | >115 | 3.0-4.0 | >75 |
| | | A1 | | | | |
| | Inhibitor | Bafilomycin | 1000 | >115 | 2.0-3.0 | >75 |
| | | A1 | | | | |
| | Inhibitor | Bafilomycin | 100 | 105-115 | 2.0-3.0 | 100 |
| | | A1 | | | | |
| | Inhibitor | Bafilomycin | 10 | 85-95 | 1.75-2.0 | >75 |
| | | A1 | | | | |
| | Activator | Trametinib | 1000 | 65-75 | 2.0-3.0 | >75 |
| | Activator | Trametinib | 100 | 35-45 | 1.25-1.5 | >75 |
| | Activator | Trametinib | 10 | 35-45 | 1.25-1.5 | >75 |
| | Inhibitor | 3-Methyladenine | 1000 | 35-45 | 1.25-1.5 | 100 |
| | Inhibitor | 3-Methyladenine | 100 | 45-55 | 1.5-1.75 | 100 |
| | Inhibitor | 3-Methyladenine | 10 | 45-55 | 1.5-1.75 | 100 |
| Inflammation | STAT3 Antagonist | Napabucasin | 100 | 45-55 | 1.5-1.75 | 100 |
| and Receptor | STAT3 Antagonist | Napabucasin | 10 | 35-45 | 1.0-1.25 | 100 |
| Target | STING Antagonist | H-151 | 10000 | 55-65 | 1.5-1.75 | >75 |
| | TRAF6 Antagonist | C25-140 | 1000 | 35-45 | 1.25-1.5 | 100 |
| | TRAF6 Antagonist | C25-140 | 100 | 45-55 | 1.5-1.75 | 100 |
| | TRAF6 Antagonist | C25-140 | 10 | <35 | 0.75-1.0 | 100 |
| | iKKb Agonist | Betulin | 10000 | 35-45 | 1.0-1.25 | 100 |
| | iKKb Agonist | Betulin | 10 | 45-55 | 1.25-1.5 | 100 |
| Metabolism | Glutathione | RSL3 | 1000 | 55-65 | 2.0-3.0 | >75 |
| and Stress | peroxidase Inhibitor | | | | | |
| Signaling | Stress Signaling | Anisomycin | 1000 | 65-75 | 1.75-2.0 | 50-75 |
| | activator | | | | | |
| | Stress Signaling | Anisomycin | 100 | 65-75 | 1.75-2.0 | >75 |
| | activator | | | | | |
| | Stress-Signaling | Anisomycin | 10 | <35 | 1.0-1.25 | 100 |
| | activator | | | | | |
| Proliferation | Cell Proliferation | Ixabepilone | 10 | 65-75 | 2.0-3.0 | 50-75 |
| and | Inhibitor | | | | | |
| Longevity | HDAC Inhibitor | Trichostatin A | 1000 | 55-65 | 1.75-2.0 | 50-75 |
| | Cell Proliferation | Paclitaxel | 10 | 55-65 | 1.5-1.75 | >75 |
| | Inhibitor | | | | | |
| | Cell Proliferation | AZD-5438 | 1000 | 35-45 | 1.0-1.25 | 100 |
| | Inhibitor | | | | | |
| | Cell Proliferation | AZD-5438 | 100 | 35-45 | 1.25-1.5 | 100 |
| | Inhibitor | | | | | |
| | Cell Proliferation | AZD-5438 | 10 | 45-55 | 1.5-1.75 | 100 |
| | Inhibitor | | | | | |
| | HDAC Inhibitor | Entinostat | 10000 | 35-45 | 1.25-1.5 | >75 |
| Disease | AMPA/Kainate | Diazoxide | 10000 | 35-45 | 1.0-1.25 | 100 |
| relevant | Receptor Activator | | | | | |
| | AMPA/Kainate | Diazoxide | 1000 | 35-45 | 1.25-1.5 | 100 |
| | Receptor Activator | | | | | |
| | AMPA/Kainate | Diazoxide | 100 | 45-55 | 1.5-1.75 | 100 |
| | Receptor Activator | | | | | |
| | AMPA Receptor | CX516 | 1000 | 35-45 | 1.0-1.25 | 100 |
| | Activator | | | | | |
| | AMPA Receptor | CX516 | 100 | 45-55 | 1.25-1.5 | 100 |
| | activator | | | | | |
| | AMPA Receptor | CX516 | 10 | <35 | 0.75-1.0 | 100 |
| | Activator | | | | | |
| | L-type Ca2+ channel | Bay K 8644 | 1000 | 35-45 | 1.25-1.5 | 100 |
| | activator | | | | | |
| | L-type Ca2+ channel | Bay K 8644 | 100 | 35-45 | 1.25-1.5 | 100 |
| | activator | | | | | |
| | L-type Ca2+ channel | Bay K 8644 | 10 | 35-45 | 1.25-1.5 | 100 |
| | activator | | | | | |

### Example 18: Small Molecule Compound Screen to Reveal Down-Regulators of Gigasome Production in Neuronal Monocultures

This example demonstrates that gigasome production can be differentially down-regulated from the baseline in a neuronal cell line derived from human neuroblastoma upon treatment with various compounds targeting different pathways, including Proteasome, Autophagy, Membrane Trafficking Proliferation and Longevity, Inflammation and Receptor Target, and Metabolism and Stress Signaling pathways.

Quantification and characterization of gigasome production using microscopy, while tracking cell viability/phenotype, was carried out as described in Example 17. By setting cell viability at ≥75% and gigasome counts ≤0.5-fold change compared to the vehicular DMSO control, compounds down-regulating gigasome production were identified in at least 5% and up to 29% of compound categories tested which are involved in membrane trafficking, metabolism and stress signaling, autophagy, inflammation and receptor target, or proliferation and longevity. In this small molecule screen, only the Proteasome pathway did not display compounds lowering gigasome production according to the criteria set.

A subset of these compounds displayed a dose-dependent downregulation of gigasome production (Table 21), with the top compounds involved in inhibition of exocytosis (GW4869 and EXO1) and synaptic transmission and release of synaptic vesicles (GV-58 and NMDA). A proteasome activator (Oleuropein) also showed a dose-dependent decrease in gigasome production, although the fold-change compared to DMSO ranged between 0.5-75 (Table 21). A compound that inhibits Nf-κB activation via iKKb inhibition nearly abolished gigasome production with a fold-change <0.2 compared to DMSO (10,000 nM Wedelolactone).

TABLE 21
Small molecule compounds that demonstrated ability to modulate gigasome production in neuronal cells.
Each compound is labelled with its targeted pathway, known mechanism of action of the compound, dose
used in the screen, number of gigasomes produced, fold change of the number of gigasomes produced compared
to the DMSO control, and the percentage viability. At times, multiples doses are highlighted.

| | | | | Number of | Fold Change | |
|---|---|---|---|---|---|---|
| | | Compound | Dose | Gigasomes | of Gigasomes | Viability |
| Pathway | Mechanism of Action | Name | (nM) | Produced | Produced vs. DMSO | (%) |
| N/A | N/A | DMSO | N/A | <35 | 1.0 | 100 |
| Trafficking | Exocytosis Inhibitor | GW4869 | 10000 | 5-10 | 0.25-0.5 | 100 |
| | | GW4869 | 1000 | 15-20 | 0.25-0.5 | 100 |
| | | GW4869 | 100 | 35-40 | 0.75-1.0 | 100 |
| | | GW4869 | 10 | 30-35 | 0.75-0.5 | 100 |
| | Post-Golgi | Exo1 | 10000 | 10-15 | 0.25-0.5 | 100 |
| | Exocytosis Inhibitor | Exo1 | 1000 | 10-15 | 0.25-0.5 | 100 |
| | | Exo1 | 100 | 20-25 | 0.5-0.75 | 100 |
| | | Exo1 | 10 | 35-40 | 0.75-1.0 | 100 |
| Disease | N-and P/Q-type Ca²⁺ | GV-58 | 10000 | 10-15 | 0.25-0.5 | >75 |
| Relevant | channel Agonist | GV-58 | 1000 | 20-25 | 0.75-1.0 | 100 |
| | | GV-58 | 100 | 20-25 | 0.75-1.0 | 100 |
| | | GV-58 | 10 | 25-30 | 0.75-1.0 | 100 |
| | NMDA Receptor | NMDA | 10000 | 15-20 | 0.25-0.5 | 100 |
| | Activator | NMDA | 1000 | 20-25 | 0.5-0.75 | 100 |
| | | NMDA | 100 | 30-35 | 0.75-1.0 | 100 |
| | | NMDA | 10 | 30-35 | 0.75-1.0 | 100 |
| | NOTCH Inhibitor | Semagacestat | 10000 | 20-25 | 0.5-0.75 | 100 |
| | | Semagacestat | 1000 | 20-25 | 0.5-0.75 | 100 |
| | | Semagacestat | 100 | 35-40 | 0.75-1.0 | 100 |
| | | Semagacestat | 10 | >40 | 0.75-1.0 | 100 |
| | BACE1 Inhibitor | Verubecestat | 10000 | 20-25 | 0.5-0.75 | 100 |
| | | Verubecestat | 1000 | 20-25 | 0.5-0.75 | 100 |
| | | Verubecestat | 100 | 25-30 | 0.5-0.75 | 100 |
| | | Verubecestat | 10 | 35-40 | 0.75-1.0 | 100 |
| Metabolism | Stress Signaling | Neflamapimod | 10000 | 15-20 | 0.5-0.75 | 100 |
| and Stress | Inhibitor | Neflamapimod | 1000 | 20-25 | 0.5-0.75 | 100 |
| Signaling | | Neflamapimod | 100 | 25-30 | 0.75-1.0 | >75 |
| | | Neflamapimod | 10 | 30-35 | 0.75-1.0 | 100 |
| | Stress signaling | SMIP004 | 10000 | 30-35 | 0.75-1.0 | 100 |
| | activator | SMIP004 | 1000 | 30-35 | 0.75-1.0 | 100 |
| | | SMIP004 | 100 | 35-40 | 0.75-1.0 | 100 |
| | | SMIP004 | 10 | >40 | >1.25 | 100 |
| | Mitochondrial | UK-5099 | 10000 | 10-15 | 0.25-0.5 | 100 |
| | pyruvate carrier | UK-5099 | 1000 | 15-20 | 0.5-0.75 | 100 |
| | inhibitor | UK-5099 | 100 | 15-20 | 0.5-0.75 | 100 |
| | | UK-5099 | 10 | 20-25 | 0.5-0.75 | 100 |
| Autophagy | Inhibitor | Hydroxychloroquine | 10000 | 20-25 | 0.75-1.0 | >75 |
| | | Hydroxychloroquine | 1000 | 25-30 | 0.75-1.0 | >75 |
| | | Hydroxychloroquine | 100 | 20-25 | 0.75-1.0 | 100 |
| | | Hydroxychloroquine | 10 | 30-35 | 1.0-1.25 | 100 |
| Inflammation | IL-1B/NLRP3 | MCC950 sodium | 10000 | 25-30 | 0.75-1.0 | 100 |
| and Receptor | antagonist | MCC950 sodium | 1000 | 20-25 | 0.5-0.75 | 100 |
| Target | | MCC950 sodium | 100 | 35-40 | 1.0-1.25 | >75 |
| | | MCC950 sodium | 10 | 35-40 | 1.0-1.25 | 100 |
| Proliferation | Sirtuin Activator | OSS_128167 | 10000 | 20-25 | 0.5-0.75 | 100 |
| and | | OSS128167 | 1000 | 25-30 | 0.75-1.0 | 100 |
| Longevity | | OSS128167 | 100 | 25-30 | 0.75-1.0 | >75 |
| | | OSS128167 | 10 | >40 | 1.0-1.25 | 100 |
| | Cell proliferation | Seliciclib | 10000 | 15-20 | 0.5-0.75 | 100 |
| | Inhibitor | Seliciclib | 1000 | 25-30 | 0.75-1.0 | 100 |
| | | Seliciclib | 100 | 25-30 | 0.75-1.0 | 100 |
| | | Seliciclib | 10 | 25-30 | 0.75-1.0 | 100 |
| Proteasome | Activator | Oleuropein | 10000 | 25-30 | 0.5-0.75 | 100 |
| | | Oleuropein | 1000 | 30-35 | 0.75-1.0 | >75 |
| | | Oleuropein | 100 | 30-35 | 0.75-1.0 | 100 |
| | | Oleuropein | 10 | >40 | 1.0-1.25 | 100 |

### Example 19: Small Molecule Compound Screen to Reveal Up-Regulators of Gigasome Production in Cardiomyocytic Monocultures

This example demonstrates that gigasome production can be differentially up-regulated from the baseline in a cardiomyocytic cell line derived from human ventricular heart tissue upon treatment with compounds encompassing different pathways, including Membrane Trafficking, Proteasome, Proliferation and Longevity, Inflammation and Receptor Target, Autophagy, and Metabolism and Stress Signaling.

Characterization and analysis of gigasome production using microscopy in Cardiomyocytes was conducted as described above for neuroblastoma cells (Example 17). Cells were stained with Hoechst 34580, CellTracker Green/CellMask Green Actin and MitoTracker Deep Red to detect nuclear material, cytoplasm/F-actin, and mitochondria, respectively, and cultured in poly-L-lysine-coated 96-well glass bottom plates at 15,800 cells/cm² and allowed to adhere overnight. For the screening, a total of 100 randomly organized compounds (TargetMol) were divided in 5 groups. Each group included an untreated condition, a vehicular DMSO control, and a known high-gigasome producing condition to comparatively study gigasome production rates. As a cell death control group, 500 nM Staurosporine was added to induce apoptosis. Cultures were incubated for 24 hours under gigasome inducing conditions at concentrations ranging between 10 nM and 10,000 nM. Before the end of the treatment, live neuronal cultures were imaged. The Zeiss ZEN Blue software was used to distribute imaging fields of view (positions) to create an unbiased imaging sample of a well. 5 images were taken using a Zeiss LSM 900 confocal microscope. Images were in 16-bit format, and contained at least an ESID (brightfield) channel, a Hoechst 34580 fluorescence channel, a CellTracker Green/Cell Mask Green Actin fluorescence channel, and a MitoTracker Deep Red fluorescence channel. The BioApps ZEN software package was used to perform cell nucleus segmentation and counting. After 24 hours, gigasomes were harvested from the cell culture media by gently removing from the top of each 96-well ⅔ of the total volume without disturbing the cell layer (containing the settled gigasomes) and by adding to each well the same amount of PBS containing 0.5 mM EDTA. The wells were washed twice gently, and the gigasome-containing media/0.5 mM EDTA PBS wash was transferred into the well of a 384-well glass bottom plate. Cells remaining in the 96-well plates were assessed for viability using CellTiter-Glo (Promega).

For gigasome analysis in 384-well plates, the Zeiss ZEN Blue software was used to distribute positions to create an unbiased imaging sample of a well. 6 images per well were taken using a Zeiss LSM 900 confocal microscope. Images were in 16-bit format, and contained at least an ESID (brightfield) channel, a Hoechst 34580 fluorescence channel, a CellTracker Green/Cell Mask Green Actin fluorescence channel, and a MitoTracker Deep Red fluorescence channel. The CellTracker Green/Cell Mask Green fluorescence channel threshold was set between 5403 and 65535 for 16-bit images. A watershed binary segmentation step was performed to separate grouped objects. For each particle in the mask, characteristic parameters of the particle were calculated and saved. In some experiments, these parameters were the particles area, circularity, mean fluorescence signal in the Hoechst 34580 channel (nuclear signal), and mean fluorescence signal in the MitoTracker Deep Red channel (mitochondrial signal). Particles of interest were identified by thresholding by area and circularity. Area was restricted to be between 7.07 and 314 μm² (which correspond to spheres with diameters ranging from 3 to 20 μm). Circularity, defined by the formula 4pi(area/perimeter\hat{ }2, was set to be between 0.8, where a value of 1.0 is a perfect circle, and a value of 0 is a line. Gigasomes were identified from the particles of interest by thresholding by mean nuclear signal. The nuclear signal threshold was set using a value equal to one quarter of a threshold value calculated by the Li auto-threshold algorithm applied to the distribution of all particles of interest. Particles of interest with nuclear signal below this value were considered “negative” for nuclear material and were identified as gigasomes. A sampling factor was calculated to extrapolate the total quantity of a type of object (particles, particles of interest, gigasomes) in the well. The sampling factor was the percentage of the well's area that was imaged. Dividing a quantity or distribution of objects by the sampling factor resulted in an estimated quantity or distribution of objects for the whole well. In some experiments, the number of cells in the cell cultures were estimated with a cell counter. Dividing a quantity or distribution of objects by the number of cells from the culture that produced them resulted in a normalized quantity or distribution. The normalized quantity or distribution of objects was described as a quantity or distribution of objects per 1000 cells.

Cardiomyocyte cultures treated with compounds targeting different pathways displayed differential rates of gigasome production. By setting cell viability at ≥75% and gigasome counts ≥1.5 fold compared to the vehicular DMSO control, gigasome producers were identified in at least 5% and up to 20% of compound categories tested which are involved in proteasome, autophagy, inflammation and receptor target, or proliferation and longevity. In this small molecule screen on cardiomyocyte cells, compounds falling in the pathways of Membrane Trafficking and Metabolism and Stress signaling, showed either some toxicity at higher doses (50-75% viability) or induced a <1.5 fold-increase in gigasome production.

The top compounds identified increasing gigasome production up to 2-fold compared to DMSO (Table 22, bold text) played a role in epigenetic regulation via HDAC inhibition (100 nM Entinostat), inhibition of late-stage Autophagy (1000-10000 nM Bafilomycin A1). Additional compounds increasing gigasome production up to 1.75-fold compared to DMSO (Table 22, bold text) included compounds falling in the Disease Relevant pathway regulating calcium handling via CaMK-II inhibition (10000 nM KN-93) and proteolytic processing via BACE1 inhibition (1000 nM LY288672).

TABLE 22
Small molecule compounds that demonstrated ability to modulate gigasome production in cardiomyocytes.
Each compound is labelled with its targeted pathway, known mechanism of action of the compound, dose
used in the screen, number of gigasomes produced, fold change of the number of gigasomes produced compared
to the DMSO control, and the percentage viability. At times, multiples doses are highlighted.

| | | | | Number of | Fold Change | |
|---|---|---|---|---|---|---|
| | | Compound | Dose | Gigasomes | of Gigasomes | Viability |
| Pathway | Mechanism of Action | Name | (nM) | Produced | Produced vs. DMSO | (%) |
| N/A | N/A | DMSO | N/A | <150 | 1.0 | 100 |
| Proteasome | Inhibitor | Tripterin | 1000 | 375-400 | 3.0-4.0 | 50-75 |
| | Inhibitor | Tripterin | 100 | <150 | 0.75-1.0 | >75 |
| | Inhibitor | MG-132 | 100 | >400 | >4.0 | 50-75 |
| | Inhibitor | MG-132 | 10 | 150-175 | 1.5-1.75 | 100 |
| Disease | CaMK-II Inhibitor | KN-93 | 10000 | 250-275 | 1.5-1.75 | >75 |
| Relevant | CaMK-II Inhibitor | KN-93 | 1000 | 250-275 | 1.25-1.5 | >75 |
| | CaMK-II Inhibitor | KN-93 | 10 | 225-250 | 1.25-1.5 | >75 |
| | CaMK-II Activator | Methyl cinnamate | 1000 | 150-175 | 1.25-1.5 | >75 |
| | NMDA Receptor | Quinolinic acid | 100 | 150-175 | 1.0-1.25 | 100 |
| | Agonist | | | | | |
| | NOTCH inhibitor | DAPT | 100 | 250-275 | 1.25-1.5 | >75 |
| | NOTCH inhibitor | DAPT | 10 | 200-225 | 1.0-1.25 | >75 |
| | BACE1 inhibitor | LY2886721 | 1000 | 175-200 | 1.5-1.75 | >75 |
| Autophagy | Inhibitor | Bafilomycin A1 | 10000 | 250-275 | 1.75-2.0 | >75 |
| | Inhibitor | Bafilomycin A1 | 1000 | 250-275 | 1.75-2.0 | >75 |
| | Inhibitor | Bafilomycin A1 | 100 | 225-250 | 1.5-1.75 | >75 |
| | Inhibitor | SAR405 | 10000 | 200-225 | 1.0-1.25 | >75 |
| | Inhibitor | SAR405 | 1000 | 150-175 | 1.0-1.25 | 100.0 |
| | Activator | Carbamazepine | 10000 | 250-275 | 1.25-1.5 | >75 |
| | Activator | Carbamazepine | 1000 | 250-275 | 1.25-1.5 | >75 |
| | Activator | Carbamazepine | 10 | 200-225 | 1.0-1.25 | >75 |
| | Activator | Dactolisib | 10000 | 150-175 | 1.25-1.5 | >75 |
| Inflammation | iKKb Agonist | Betulin | 100 | 175-200 | 1.5-1.75 | 100 |
| and Receptor | iNOS antagonist | 1400W | 10 | 150-175 | 1.25-1.5 | 100 |
| Target | | dihydrochloride | | | | |
| | LXR antagonist | GSK2033 | 10000 | 150-175 | 1.25-1.5 | 100 |
| | LXR Agonist | T0901317 | 10000 | 150-175 | 1.0-1.25 | >75 |
| | NADPH Oxidase | Apocynin | 10000 | 150-175 | 1.0-1.25 | >75 |
| | antagonist | | | | | |
| | NADPH Oxidase | Apocynin | 10 | 150-175 | 1.0-1.25 | >75 |
| | antagonist | | | | | |
| | PPARγ antagonist | T0070907 | 100 | 150-175 | 1.0-1.25 | 100 |
| | PPARγ antagonist | T0070907 | 10 | 150-175 | 1.0-1.25 | 100 |
| Proliferation | HDAC Inhibitor | Entinostat | 100 | 300-325 | 1.75-2.0 | >75 |
| and | Tyrosine | Sunitinib | 10000 | 200-225 | 1.5-1.75 | 50-75 |
| Longevity | Kinase/VEGFR, | | | | | |
| | PDGFR Inhibitor | | | | | |
| | Cell proliferation | Paclitaxel | 10 | 150-175 | 1.5-1.75 | >75 |
| | inhibitor | | | | | |
| | SIRT1 Inhibitor | Selisistat | 10 | 150-175 | 1.25-1.5 | 100 |
| | SIRT1Activator | SRT 1720 | 1000 | 150-175 | 1.0-1.25 | >75 |
| | DNA | 5-Azacytidine | 1000 | 150-175 | 1.0-1.25 | 50-75 |
| | Methyltransferase | | | | | |
| | Inhibitor | | | | | |
| Membrane | ER to Golgi | Monensin | 1000 | 250-275 | 2.0-3.0 | 50-75 |
| Trafficking | inhibitor | sodium salt | | | | |
| | ER to Golgi | Monensin | 100 | 150-175 | 1.25-1.5 | >75 |
| | inhibitor | sodium salt | | | | |
| | ER to Golgi | Monensin | 10 | 150-175 | 1.25-1.5 | >75 |
| | inhibitor | sodium salt | | | | |
| | ER to Golgi | Brefeldin A | 10 | 250-275 | 1.25-1.5 | >75 |
| | inhibitor | | | | | |
| Metabolism | Stress-Signaling | Anisomycin | 100 | 150-175 | 1.25-1.5 | 50-75 |
| and Stress | Activator | | | | | |
| Signaling | Stress-Signaling | SMIP004 | 10000 | 175-200 | 1.25-1.5 | >75 |
| | Activator | | | | | |
| | Stress-Signaling | SMIP004 | 1000 | 150-175 | 1.0-1.25 | 100 |
| | Activator | | | | | |

### Example 20: Small Molecule Compound Screen to Reveal Down-Regulators of Gigasome Production in Cardiomyocytic Monocultures

This example demonstrates that gigasome production can be differentially down-regulated from the baseline in a cardiomyocytic cell line derived from human ventricular heart tissue upon treatment with various compounds targeting different pathways, including Proteasome, Autophagy, Membrane Trafficking Proliferation and Longevity, Inflammation and Receptor Target, Disease Relevant and Metabolism and Stress Signaling pathways.

Quantification and characterization of gigasome production using microscopy, while tracking cell viability/phenotype, was carried out as described in Example 19. By setting cell viability at ≥75% and gigasome counts ≤0.5-fold change compared to the vehicular DMSO control, compounds down-regulating gigasome production were identified in at least 5% and up to 20% of compound categories tested which are involved in proteasome, metabolism and stress signaling, membrane trafficking, autophagy, inflammation and receptor target, or proliferation and longevity.

Within these pathways, various compounds also displayed a dose-dependent downregulation of gigasome production (Table 23), with the top compounds involved in inhibition of Nf-kB activation via iKKb inhibition (Wedelolactone); metabolic regulation via inhibition of Glucose Transporter 1 (WZB117) and mitochondrial function (3-Nitropropanoic acid, CGP37157 and UK-5099).

TABLE 23
Small molecule compounds that demonstrated ability to modulate gigasome production in cardiomyocytes.
Each compound is labelled with its targeted pathway, known mechanism of action of the compound, dose
used in the screen, number of gigasomes produced, fold change of the number of gigasomes produced compared
to the DMSO control, and the percentage viability. At times, multiples doses are highlighted.

| | | | | Number of | Fold Change | |
|---|---|---|---|---|---|---|
| | | Compound | Dose | Gigasomes | of Gigasomes | Viability |
| Pathway | Mechanism of Action | Name | (nM) | Produced | Produced vs. DMSO | (%) |
| Control | N/A | DMSO | N/A | <150 | 1 | 100 |
| Inflammation | iKKb | Wedelolactone | 10000 | <20 | <0.25 | >75 |
| and Receptor | antagonist | Wedelolactone | 1000 | 50-75 | 0.5-0.75 | >75 |
| Target | | Wedelolactone | 100 | 75-90 | 0.5-0.75 | 100.0 |
| | | Wedelolactone | 10 | 75-90 | 0.5-0.75 | 100.0 |
| | iNOS | 1400W | 10000 | 50-75 | 0.5-0.75 | 100.0 |
| | antagonist | dihydrochloride | | | | |
| | | 1400W | 1000 | 90-105 | 0.75-1.0 | 100.0 |
| | | dihydrochloride | | | | |
| | | 1400W | 100 | 90-105 | 0.75-1.0 | 100.0 |
| | | dihydrochloride | | | | |
| | | 1400W | 10 | >150 | 1.25-1.5 | 100.0 |
| | | dihydrochloride | | | | |
| | Histamine H2 | Dimaprit | 10000 | 50-75 | 0.25-0.5 | 100.0 |
| | Receptor | dihydrochloride | | | | |
| | agonist | Dimaprit | 1000 | 50-75 | 0.5-0.75 | >75 |
| | | dihydrochloride | | | | |
| | | Dimaprit | 100 | 75-90 | 0.75-1.0 | 100.0 |
| | | dihydrochloride | | | | |
| | | Dimaprit | 10 | 90-105 | 0.75-1.0 | 100.0 |
| | | dihydrochloride | | | | |
| Metabolism | GLUT1 | WZB117 | 10000 | 35-50 | 0.25-0.5 | >75 |
| and Stress | Inhibitor | WZB117 | 1000 | 105-120 | 0.75-1.0 | >75 |
| Signaling | | WZB117 | 100 | 105-120 | 0.75-1.0 | 100.0 |
| | | WZB117 | 10 | 120-135 | 1.0-1.25 | 100.0 |
| | Succinate | 3-Nitropropanoic | 10000 | 35-50 | 0.25-0.5 | 100.0 |
| | Dehydrogenase | acid | | | | |
| | Inhibitor | 3-Nitropropanoic | 1000 | 50-75 | 0.25-0.5 | >75 |
| | | acid | | | | |
| | | 3-Nitropropanoic | 100 | 90-105 | 0.75-1.0 | 100.0 |
| | | acid | | | | |
| | | 3-Nitropropanoic | 10 | 105-120 | 0.75-1.0 | >75 |
| | | acid | | | | |
| | Mitochondrial | CGP37157 | 10000 | 50-75 | 0.25-0.5 | 100.0 |
| | Na⁺/Ca²⁺ | CGP37157 | 1000 | 50-75 | 0.25-0.5 | >75 |
| | exchanger | CGP37157 | 100 | 105-120 | 0.75-1.0 | 100.0 |
| | Inhibitor | CGP37157 | 10 | 120-135 | 0.75-1.0 | 100.0 |
| | Acetyl-CoA | TOFA | 10000 | 75-90 | 0.75-1.0 | 100.0 |
| | Carboxylase | TOFA | 1000 | 105-120 | 0.75-1.0 | >75 |
| | Inhibitor | TOFA | 100 | 105-120 | 1.0-1.25 | 100.0 |
| | | TOFA | 10 | 120-135 | 1.0-1.25 | >75 |
| | Stress | Neflamapimod | 10000 | 75-90 | 0.75-1.0 | 100.0 |
| | Signaling | Neflamapimod | 1000 | 75-90 | 0.75-1.0 | >75 |
| | Inhibitor | Neflamapimod | 100 | 90-105 | 0.75-1.0 | 100.0 |
| | | Neflamapimod | 10 | 120-135 | 1.0-1.25 | 100.0 |
| Autophagy | Inhibitor | 3-Methyladenine | 10000 | 90-105 | 0.5-0.75 | >75 |
| | | 3-Methyladenine | 1000 | 120-135 | 0.75-1.0 | >75 |
| | | 3-Methyladenine | 100 | >150 | 1.25-1.5 | >75 |
| | | 3-Methyladenine | 10 | >150 | 1.25-1.5 | >75 |
| | Inhibitor | Spautin-1 | 10000 | 75-90 | 0.5-0.75 | 100.0 |
| | Inhibitor | Spautin-1 | 1000 | 90-105 | 0.5-0.75 | 100.0 |
| | Inhibitor | Spautin-1 | 100 | 120-135 | 0.75-1.0 | 100.0 |
| | Inhibitor | Spautin-1 | 10 | 105-120 | 0.75-1.0 | 100.0 |
| Disease | L-type Ca2+ | Bay K 8644 | 10000 | 75-90 | 0.25-0.5 | >75 |
| Relevant | channel | Bay K 8644 | 1000 | 105-120 | 0.75-1.0 | >75 |
| | activator | Bay K 8644 | 100 | 120-135 | 0.5-0.75 | >75 |
| | | Bay K 8644 | 10 | >150 | 0.75-1.0 | 100.0 |

### Example 21: Modulation of Macrophages by Exogenously Applied Gigasomes

Macrophages are known to be capable of phagocytosing a variety of material. Phagocytosis is also known to be capable of modulating the profile or function of the macrophage. This example demonstrates that exogenously applied gigasomes are bound and internalized by macrophages—and that exogenously applied gigasomes modulate macrophages in a way that was distinct from exogenously applied apoptotic (dead) bodies or apoptotic cells.

### Gigasome Production and Harvest

To produce gigasomes, human cardiomyocyte cells were collected and resuspended in either serum free DMEM-F12 or RPMI-1640 media and stained with a nuclear marker, and cytosolic stain with or without a pH sensitive cytosolic stain (Table 24) for 12 to 30 minutes at 37° C. Cells were then centrifuged at 300 g, 5 minutes, and resuspended in cardiomyocyte medium (DMEM-F12 Medium (Gibco), 10% Fetal Bovine Serum (Gibco) and 1% P/S (Fisher)) and plated at 15.8 k cells/cm². The next day, media was removed from cells and replaced with cardiomyocyte medium containing 31 nM Bafilomycin A1 (MilliporeSigma) and 10 nM AZD-2014 (Selleck Chemicals), or 200 nM Rapamycin (MilliporeSigma) for 24 h. To generate apoptotic bodies, cells were treated with 500 nM Staurosporine (Sigma S6942) for 24 h, and to generate apoptotic cells, fresh cardiomyocyte medium was added for 19 h, then 500 nM Staurosporine was added for 5 h. Gigasomes and apoptotic bodies were collected by harvesting the media supernatant, washing the cells once with PBS containing 0.5 mM EDTA, then pooling the wash with the supernatant. Apoptotic cells were collected using the same procedure pipetting the media across cells to ensure detachment. Collections were centrifuged for 5 minutes at 4° C. at 300 g. To isolate and enrich gigasomes and apoptotic bodies, the supernatant was collected. To isolate and enrich apoptotic cells, the supernatant from the 300 g spin was removed, and cells were resuspended in PBS. Gigasomes, apoptotic bodies, and apoptotic cells were centrifuged for 5 minutes at 4° C. at 1000 g. This supernatant was discarded and gigasomes, apoptotic bodies, and apoptotic cells were washed by resuspending in PBS and centrifuging for 5 minutes at 4° C. at 1000 g. This supernatant was discarded, and gigasomes, apoptotic bodies, and apoptotic cells were resuspended in macrophage Medium (RPMI-1640 with 1% Penicillin-Streptomycin, Beta-mercaptoethanol (50 uM), 10% FBS).

TABLE 24
Cellular stains used in experiments

| Stain/Marker | Type | Target | Supplier |
|---|---|---|---|
| Hoechst-33342 | Nuclear Stain | DNA | Invitrogen |
| Celltracker | Cytosolic Stain | Cellular Amines | ThermoFisher |
| Green CMFDA | | | |
| phRODO Deep | pH-sensitive | Cellular Amines | ThermoFisher |
| Red TFP | cytosolic stain | | |

### Gigasome Imaging and Quantification

Gigasomes, apoptotic bodies, and apoptotic cells were quantified by diluting media suspensions in PBS in an imaging multi-well plate. The Zeiss ZEN Blue software was used to distribute imaging fields of view (positions) to create an unbiased imaging sample of a well. Images were taken using a Zeiss LSM 900 confocal microscope. Images were in 16-bit format, and contained at least an ESID (brightfield) channel, a Nuclear fluorescence channel (Hoechst 33342, Table 24), and a cytosolic fluorescence channel (Celltracker Green, Table 24). 5 images per well were captured. ImageJ was used to impose a threshold to create a mask of objects in the field of view. The Cytosolic fluorescence channel was thresholded between 5403 and 65535 for 16-bit images. A watershed binary segmentation step was performed to separate grouped objects. For each particle in the mask, characteristic parameters of the particle were calculated and saved. In some experiments, these parameters were the particles area, circularity, mean fluorescence signal in the Hoechst 33342 channel (nuclear signal). Particles of interest were identified by thresholding by area and circularity. A threshold was set by considering the background fluorescence signal from an unstained control population of gigasomes. Celltracker Green—positive particles were totaled for each particle type and used to calculate the concentration of particles in the media suspension.

### Exogenous Gigasome Application to Macrophages

Human monocytes were seeded at 31,250 to 62,500 cells/cm² in macrophage medium with 100 ng/mL Phorbol 12-Myristate 13-Acetate (Sigma, P8139) for macrophage differentiation in 48- or 96-well tissue culture or glass bottom plates. 48 hours later, media was removed and replaced with macrophage media for an additional 24 h. Gigasome, apoptotic body, or apoptotic cell suspensions were diluted and added to macrophages to attain ratios of 2:1 up to 10:1 including 3:1, 4:1, 5:1, 6:1, 7:1, 8:1, and 9:1 particles/macrophage. Macrophage culture plates were centrifuged at 195 g (1000 rpm, H-flex 1 Rotor, ThermoFisher) for 60 seconds to speed particle settling and interaction with macrophages. Macrophages with or without particles added were cultured at 370° C. for at least 4 hours.

### Imaging of Gigasome Phagocytosis

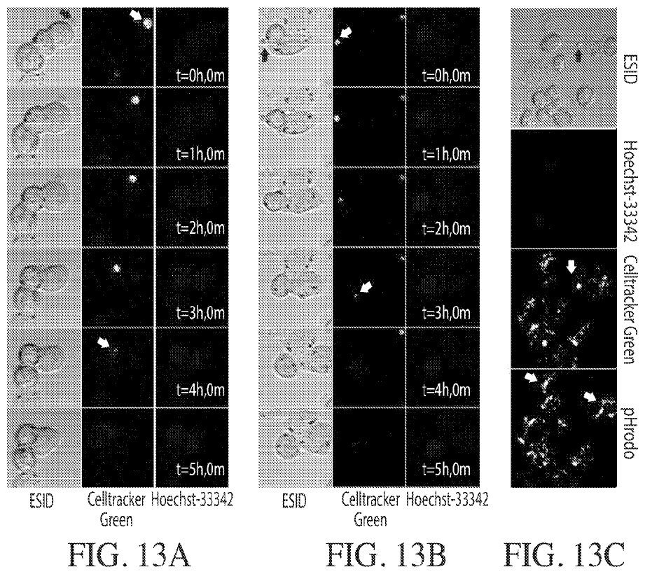

For some experiments, gigasomes were added to unstained macrophages and live-imaged using the Zeiss LSM-900 microscope in a growth chamber with controlled temperature (37° C.) and CO₂ (5%) for a total of 24 h immediately following gigasome application, imaging every 30 minutes. Macrophages were observed phagocytosing (internalizing) Celltracker-Green-positive, Hoechst-negative Gigasomes which results in their digestion and loss of Celltracker green fluorescence (FIG. 13A-B).

In some experiments, gigasomes were additionally labeled with the pH-sensitive dye, phRODO, which is nonfluorescent in pH neutral environments (pH ˜7), and fluorescent in acidic (pH ˜4-6) environments. This feature allows specific tracking of macrophage gigasome phagocytosis. Gigasomes untouched by macrophages or only in surface contact remain at a neutral pH and lack pHrodo signal, whereas those that have been phagocytosed enter the acidic environment of the macrophage lysosome and have active pHrodo fluorescence. 4 hours post gigasome addition macrophage cell culture plates were gently tapped for mechanical dissociation then thoroughly rinsed with PBS to remove the majority of excess unbound gigasomes. Fresh macrophage media was added, and macrophages were imaged at a single time point 4 hours post gigasome addition. Some gigasomes could be observed apart from macrophages or at the macrophage surface with remaining strong Celltracker green fluorescence and lacking pHrodo fluorescence (FIG. 13C). Macrophages had surface-bound uninternalized gigasomes and/or phagocytosed gigasomes inducing pHrodo signal (FIG. 13C), indicating gigasomes are readily bound and often phagocytosed by macrophages.

### Macrophage Media Supernatant and RNA Collection for Cytokine or Transcriptome Measurement

Four hours following exogenous application of gigasomes, apoptotic bodies, or apoptotic cells, macrophage cell culture plates were gently tapped for mechanical dissociation then thoroughly rinsed with PBS to remove the majority of excess unbound particles. Macrophage media with or without 10 ng/mL Lipopolysaccharide (LPS, Sigma, L4391) was added to allow the study of particle influence on macrophage phenotypes such as cytokine release in a basal state or model pro-inflammatory environment, respectively. For studies characterizing the macrophage transcriptome responses to particle addition, macrophages were cultured for 4 additional hours, then total RNA was collected by removing media and adding RNAshield buffer (Zymo) and before freezing at −20° C. For experiments assessing cytokine production, cells were incubated for 24 hours total post LPS/control media administration. Media was collected and centrifuged at 1000 g, 2 minutes. Supernatants were saved at −20° C.

### Impact of Exogenous Gigasome Application on Macrophage Transcriptomic Profile

Total RNA from treated macrophages samples saved in RNAshield buffer were extracted (Zymo DNA/RNA Nano kit (NC1631290, Fisher)). PolyA isolation, cDNA generation, library construction, indexing, and spot QC were performed according to the Illumina TrueSeq RNAseq pipeline, sequenced on the Illumina NextSeq, and paired-end 50 bp reads aligned to the hg19 human genome. Normalized counts were analyzed comparing macrophages treated with 8 gigasomes per macrophage, or left untreated. Gene Set Enrichment Analyses were performed using the KEGG database. Select significantly enriched pathways (Discovery-based uncorrected p-value for GSEA<0.05) that were upregulated in gigasome-treated macrophages with significantly upregulated member genes with fold change >1.5 are presented in Table 25. Note slight gene redundancies across pathways due to functional overlaps. At this time point following gigasome addition (4 h gigasome addition followed by a washout and 4 additional hours), gigasomes drove a proinflammatory transcriptional program spanning several KEGG functional categories including signatures of TNF-α/NF-κB-like signaling, cytokine-cytokine receptor interactions, inflammatory responses, and complement system activation. Thus, gigasome treatment can be used to provoke an overall proinflammatory response in macrophages.

TABLE 25
Select upregulated pathways, pathway Net Enrichment Scores (NES), and lists of member
genes significantly upregulated (>1.5fold, p < 0.05) with gigasome treatment.

| Upregulated KEGG Pathway | NES | Upregulated Genes |
|---|---|---|
| HALLMARK_TNFA_SIG- | 1.948 | INHBA, SERPINB2, CSF2, CCN1, IL6, IL7R, IER3, |
| NALING_VIA_NFKB | | SGK1, PTX3, PTGS2, SERPINE1, GFPT2, SOD2, |
| | | BIRC3, CXCL6, BCL3, CCL2, SAT1, KYNU, MAFF, |
| | | PPP1R15A, JUNB, ATF3, GADD45B, BHLHE40, |
| | | DUSP1, LITAF, DNAJB4, CEBPB, GEM, PTPRE, |
| | | TGIF1, RCAN1, BIRC2, HES1, PLAUR, TSC22D1 |
| KEGG_CYTOKINE_CY- | 1.623 | INHBA, CCL26, CCR7, CSF2, IL24, IL6, LIFR, IL15, |
| TOKINE_RECEPTOR_IN- | | IL11, IL7R, CCL15, CXCL12, CXCR4, EGFR, |
| TERACTION | | CXCL6, CCL2, CXCL8, TNFRSF10D |
| HALLMARK_INFLAM- | 1.297 | INHBA, HAS2, CCR7, IL6, IL15, IL7R, SERPINE1, |
| MATORY_RESPONSE | | EBI3, CXCL6, CCL2, CXCL8, C5AR1, SELENOS, |
| | | PTPRE, SLC31A2, RGS1, CD82, RGS16, PLAUR |
| HALLMARK_COMPLEMENT | 1.296 | PCSK9, SERPINB2, CIR, CDH13, IL6, PLAT, |
| | | SERPINE1, PIM1, PRSS3, KYNU, MAFF, HSPA1A, |
| | | CLU, S100A13, CTSV, LGALS3, CEBPB, IRF2, |
| | | APOC1, TFPI2, CPM, PLAUR |

### Impact of Exogenous Gigasome Application on Macrophage Cytokine Secretion

To extend and validate the inflammatory profile observed with transcriptomics, cytokine secretion was measured in macrophages treated with gigasomes for 4 h which were then washed out and macrophages were treated with or without LPS for 24 hours. Media supernatants were collected and analyzed with the ELLA system (ProteinSimple) or standard ELISA Kits (IL-10—Invitrogen #88-7106-22). 32 samples×8 analyte ELLA cartridges measured analytes including IL-1β, TNF-α, IL-6, GM-CSF, and IL-8. Samples were diluted to conform analytes to the range of detection in respective dilution buffers, and ELLA analyses or ELISA's performed according to manufacturer protocols. Values below the limit of detection were set at the lower limit of detection for purposes of analysis.

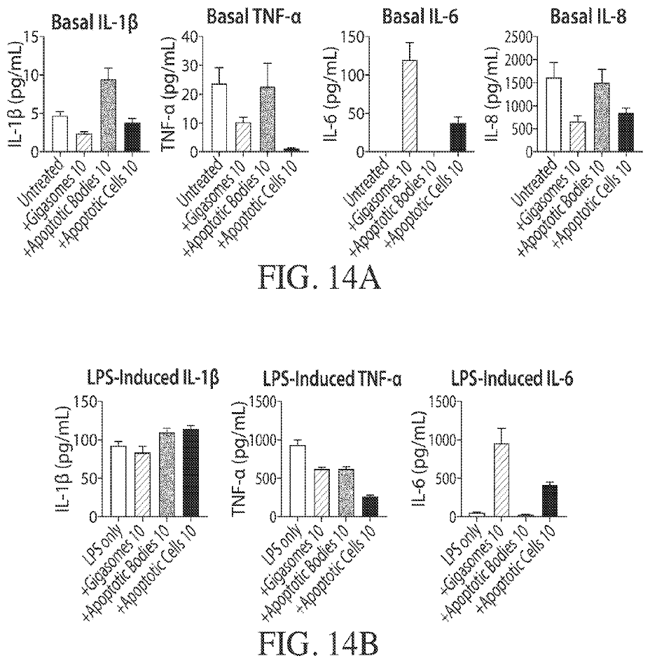

Compared to untreated samples, a high dose of gigasomes (10 gigasomes/macrophage) modestly reduced the low basal levels of some cytokines IL-1β, TNF-α, and IL-8, and increased others including IL-6 (FIG. 14A). In contrast, Apoptotic bodies increased basal levels of IL-1β, and did not affect basal TNF-α, IL-6, or IL-8 (FIG. 14A). Apoptotic cells decreased basal levels of IL-1β, TNF-α, and IL-8, and increased IL-6 (FIG. 14A). In the context of a proinflammatory stimulus, LPS, gigasomes modestly reduced TNF-α, did not affect IL-1β, and increased IL-6 (FIG. 14B). In contrast, apoptotic bodies increased LPS-induced IL-1β, reduced TNF-α, and did not impact IL-6 (FIG. 14B). Apoptotic cells increased LPS-induced IL-1β and IL-6, and reduced TNF-α (FIG. 14B).

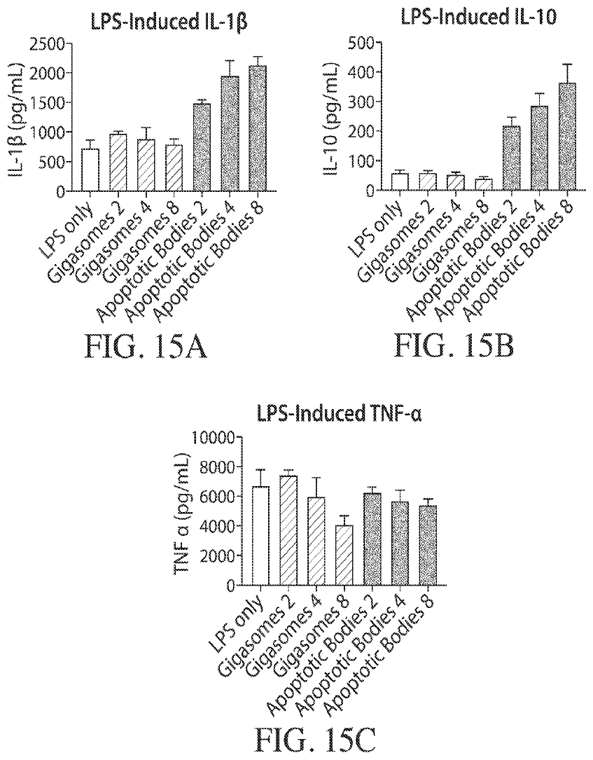

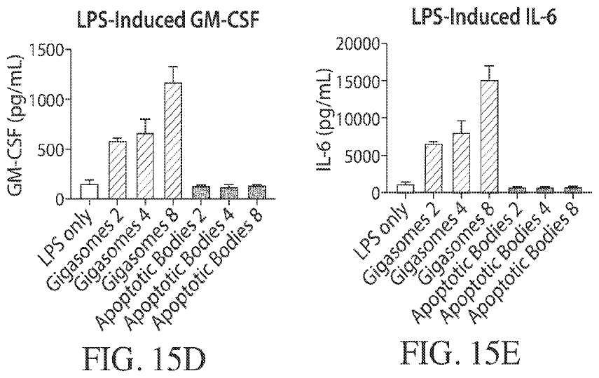

Next, the dose-dependent effect of exogenously applied gigasomes or apoptotic bodies on LPS-induced cytokine production was further examined. Gigasomes impacted neither LPS-induced IL-1β nor IL-10, whereas apoptotic bodies dose-dependently increased both (FIG. 15A-B). Gigasomes also mildly reduced TNF-α only at higher doses whereas apoptotic bodies had a negligible effect (FIG. 15C). Conversely, gigasomes dose-dependently increased LPS-stimulated IL-6 and GM-CSF, but apoptotic bodies had no effect (FIG. 15D-E). These data suggest dose-dependent and largely distinct macrophage inflammatory phenotypes induced by gigasome versus apoptotic body application. Further, the observed increases in IL-6 and GM-CSF with 8 gigasomes added per macrophage are consistent with observed increases in mRNA levels observed at the mRNA level in the transcriptome experiment (IL6 and CSF2 genes, respectively). Overall, gigasome application modulates the production of key inflammatory cytokines when compared to either untreated samples or apoptotic bodies.

What is claimed is:

1. A method of making or manufacturing a gigasome preparation, comprising:
providing a volume comprising:
(i) a population of producer cells, wherein the producer cells are human cells; and
(ii) a medium;
maintaining (e.g., culturing) the population of producer cells under conditions that allow for exopheresis, wherein the producer cells are viable after the exopheresis, and
enriching membrane-bound bodies on the basis of having a diameter between about 1-20 μm from the volume (e.g., from the medium),
thereby making or manufacturing the gigasome preparation.

2. A method of inducing release, from a population of producer cells, of membrane-bound bodies comprising nonessential products from the population of producer cells, comprising:
providing a volume comprising:
(i) a population of producer cells, wherein the producer cells are human cells; and
(ii) a medium;
maintaining (e.g., culturing) the population of producer cells under conditions that allow for release of membrane-bound bodies from the producer cells, wherein the membrane-bound bodies comprise one or more products nonessential to the producer cells; and
enriching membrane-bound bodies on the basis of comprising the one or more nonessential products (e.g., from the medium),
thereby inducing release of membrane-bound bodies comprising nonessential products from the population of producer cells;
optionally wherein the membrane-bound bodies comprises organelles (e.g., mitochondria, e.g., dysfunctional mitochondria, or lysosomes), protein aggregates, lipids, protein translation machinery, ribosomes, cytoplasm or nonessential components or constituents thereof, nonessential metabolites, nonessential small molecules, nonessential nucleic acid molecules (e.g., mRNAs, miRNAs, or siRNAs), or nonessential carbohydrates (e.g., sugars or glycans); and
optionally wherein the membrane-bound bodies have diameters between about 1-20 μm.

3. The method of claim 1 or 2, wherein the method is performed in vitro.

4. The method of claim 1 or 2, wherein the method is performed ex vivo.

5. The method of any of claims 1-4, wherein the maintaining is under conditions whereby a plurality of the producer cells in the preparation remain viable after the maintaining, e.g., a plurality of the producer cells do not undergo cell death (e.g., apoptosis or necrosis).

6. The method of any of claims 1-5, wherein the population of producer cells is stressed compared to a reference cell (e.g., an otherwise similar cell that is not maintained under conditions that allow for exopheresis and/or conditions that allow for release of membrane-bound bodies from the producer cells, wherein the membrane-bound bodies comprise one or more nonessential products).

7. The method of claim 6, wherein the producer cell stress is proteotoxic stress.

8. The method of claim 6, wherein the producer cell has impaired autophagy.

9. The method of claim 6, wherein the producer cell has higher levels of autophagy relative to an otherwise similar cell that is not maintained under conditions that allow for exopheresis and/or conditions that allow for release of membrane-bound bodies from the producer cells, wherein the membrane-bound bodies comprise one or more nonessential products.

10. The method of claim 9, wherein the higher levels of autophagy result in the membrane-bound bodies comprising higher levels of LC3-II relative to the producer cell.

11. The method of of claim 6, wherein the producer cell has a downregulated mTOR pathway relative to an otherwise similar cell that is not maintained under conditions that allow for exopheresis and/or conditions that allow for release of membrane-bound bodies from the producer cells, wherein the membrane-bound bodies comprise one or more nonessential products.

12. The method of claim 6, wherein the producer cell has a higher metabolic activity than an otherwise similar cell that is not maintained under conditions that allow for exopheresis and/or conditions that allow for release of membrane-bound bodies from the producer cells, wherein the membrane-bound bodies comprise one or more nonessential products.

13. The method of any of the preceding claims, wherein the maintaining is under conditions whereby no more than 10%, 20%, 30%, 40%, or 50% of the producer cells of the population undergo cell death (e.g., apoptosis or necrosis), e.g., over a period of 6, 12, 24, 36, 48, 60, or 72 hours.

14. The method of any of the preceding claims, wherein at least 50%, 60%, 70%, 80%, 90%, 95%, or 100% of the producer cells of the population remain viable after exopheresis.

15. The method of any of the preceding claims, wherein at least 50%, 60%, 70%, 80%, 90%, 95%, or 100% of the producer cells of the population do not comprise detectable levels of an apoptotic marker after exopheresis.

16. The method of any of the preceding claims, wherein at least 50%, 60%, 70%, 80%, 90%, 95%, or 100% of the producer cells of the population are negative for apoptosis according to an apoptosis assay, e.g., a TUNEL assay or an annexin V assay.

17. The method of any of the preceding claims, wherein at least 50%, 60%, 70%, 80%, 90%, 95%, or 100% of the producer cells of the population do not comprise increased levels of an apoptotic marker after exopheresis relative to an otherwise identical producer cell prior to exopheresis.

18. The method of any of claims 15-17, wherein the apoptotic marker comprises increased caspase (e.g., caspase-3) activity, DNA degradation (e.g., as determined by a TUNEL assay), or surface-exposed phosphatidylserine (e.g., as determined by an annexin V assay).

19. The method of any of the preceding claims, wherein the maintaining comprises incubating the producer cells under conditions suitable for inducing production of gigasomes from a plurality of the producer cells of the population (e.g., inducing the production of about 1, 2, 3, 4, or 5 gigasomes or membrane-bound bodies per producer cell of the plurality).

20. The method of any of the preceding claims, wherein the maintaining comprises incubating the producer cells under conditions suitable for continuous production of gigasomes or membrane-bound bodies (e.g., wherein each producer cell produces at least about 2, 3, 4, 5, 6, 7, 8, 9, or 10 gigasomes or membrane-bound bodies, e.g., over the course of at least 1, 2, 3, 4, 5, 6, 7, 8, 9, or 10 days).

21. The method of any of the preceding claims, wherein the producer cells are maintained (e.g., cultured) in a monoculture.

22. The method of any of the preceding claims, wherein the producer cell is selected from a neuron (e.g., a HCN2 cell, or a HT22 cell), a neuroblastoma cell (e.g., an SH-SY5Y cell), a neural progenitor cell, a muscle cell, (e.g., a cardiac muscle cell), a stem cell (e.g., an induced pluripotent stem cell (iPSC)), an endothelial cell (e.g., a microvascular endothelial cell, e.g., a cerebral microvascular endothelial cell), HBEC-5i, ReNcell CX, or iCell GlutaNeurons.

23. The method of claim 21 or 22, wherein the producer cells are primary cells (e.g., neuronal cells, neural progenitor cells, muscle cells (e.g., cardiac muscle cells), endothelial cells, or stem cells).

24. The method of any of the preceding claims, wherein the producer cells are maintained (e.g., cultured) with a second cell type (e.g., in co-culture).

25. The method of claim 24, wherein the second cell type is selected from macrophages (e.g., THP-1) and microglia (e.g., iCell Microglia, Huμglia, CHME-5, HMO6, and HMC3).

26. The method of claim 24, wherein the producer cells and the second cell type (e.g., macrophages) are physically separated (e.g., transwell or separation insert).

27. The method of any of the preceding claims, wherein the producer cells are maintained in an organoid system.

28. The method of any of the preceding claims, wherein each producer cell produces, on average, at least 2, 3, 4, 5, 6, 7, 8, 9, or 10 gigasomes or membrane-bound bodies.

29. The method of any of the preceding claims, wherein the method yields at least 1, 10, 100, 500, or 1000 gigasomes per producer cell.

30. The method of any of the preceding claims, wherein maintenance (e.g., culturing) of the producer cells further comprises adding an agent that promotes exopheresis.

31. The method of claim 30, wherein the agent is selected from a small molecule (e.g., rapamycin, isoproterenol, hydrogen peroxide, spautin-1, or MG-132, or any combination thereof) and/or an RNAi agent targeting a gene (e.g., wherein the gene is HSF1, ATG7, BECN1, LGG-1/2, UBL5, PINK1, DCT1, PDR1, MTORC1, or AKT, or any combination thereof), and/or a gene editing agent.

32. The method of any of the preceding claims, wherein, during the maintaining step, at least 75%, 80%, 85%, 90%, 95%, or 100% of the producer cells are negative for one or more apoptotic signatures, e.g., as measured using a TUNEL assay, Annexin V staining, or caspase levels or activity.

33. The method of any of the preceding claims, wherein the producer cells, after the maintaining step, comprise fewer nonessential products (e.g., organelles (e.g., mitochondria, e.g., dysfunctional mitochondria, or lysosomes), protein aggregates, and/or lipids) than before the maintaining the step.

34. The method of any of the preceding claims, wherein enriching comprises increasing the concentration of membrane-bound bodies having a diameter of 1-20 μm (e.g., gigasomes as described herein) by at least 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000-fold.

35. The method of any of the preceding claims, wherein the method further comprises loading a cargo into one or more gigasomes in the preparation.

36. A purified preparation of membrane-bound bodies (e.g., gigasomes), produced by the method of any of the preceding claims.

37. The purified preparation of claim 36, wherein the membrane-bound bodies (e.g., gigasomes) comprise a cargo, e.g., an exogenous cargo.

38. A purified preparation of membrane-bound bodies (e.g., gigasomes), wherein the membrane bound bodies of the preparation:
are about 1-20 μm in diameter,
comprise one or more human protein; and
have one or more of the following characteristics:
a) comprise an organelle (e.g., mitochondria or lysosomes)
b) comprise a product nonessential to a producer cell from which the membrane bound bodies are produced (e.g., dysfunctional mitochondria or a protein aggregate);
c) have an excitation ratio (405/476 nm) of at least about 1.2, 1.4, or 1.6, or about 1.2-1.8, 1.4-1.8, e.g., as measured using a mitoROGFP oxidation assay, e.g., as described in Melentijevic et al 2017; or
d) are enriched for LC3 and/or phosphatidylserine.

39. The purified preparation of membrane-bound bodies of claim 38, wherein the membrane-bound bodies originate from human cells.

40. The purified preparation of membrane-bound bodies of claim 39, wherein the human cells comprise neurons (e.g., HCN2 cells, or HT22 cells), neural progenitor cells, muscle cells (e.g., cardiac muscle cells), stem cells (e.g., induced pluripotent stem cells (iPSCs)), endothelial cells (e.g., microvascular endothelial cells, e.g., cerebral microvascular endothelial cells), HBEC-5i, ReNcell CX, or iCell GlutaNeurons.

41. The purified preparation of membrane-bound bodies of claim 39, wherein the human cells are primary cells (e.g., neuronal cells, neural progenitor cells, muscle cells (e.g., cardiac muscle cells), endothelial cells, or stem cells).

42. The purified preparation of membrane-bound bodies of any of claims 36-41, wherein the membrane-bound bodies or gigasomes comprise a cargo, e.g., an exogenous cargo.

43. A method of improving the health or function of a cell in a mammalian subject (e.g., human subject), the method comprising inducing exopheresis by the cell.

44. The method of claim 43, wherein the exopheresis reduces the quantity and/or concentration of a nonessential product in the cell.

45. The method of claim 44, wherein the nonessential product comprises a protein aggregate or dysfunctional mitochondria.

46. A method of delivering a cargo to a target cell, the method comprising contacting the target cell with purified preparation of claim 36 or 42 under conditions suitable for delivery of the cargo to the target cell.

47. A method of delivering membrane-bound bodies (e.g., gigasomes) to a target cell, the method comprising contacting the target cell with a purified preparation of claim 36 or 42 under conditions suitable for delivery of the membrane-bound bodies or gigasomes to the target cell.

48. The method of claim 46 or 47, wherein the target cell is situated in a subject, and the method comprises administering the gigasome to the subject.

49. The method of any of claims 46-48, wherein the gigasome was produced in vitro by a producer cell.

50. The method of claim 49, wherein the cargo is exogenous to the producer cell.

51. A method of modulating dysregulated exopheresis in a cell, the method comprising inducing or inhibiting exopheresis in the cell, e.g., by contacting the cell with an agent that induces or inhibits exopheresis.

52. A method of improving the health or function of a cell in a mammalian subject (e.g., human subject), the method comprising inducing exopheresis by the cell by contacting the cell with an agent that induces exopheresis.

53. A method of modulating the inflammatory state of a target cell, the method comprising contacting the target cell with a gigasome derived from a human cell, thereby modulating the inflammatory state of the cell.
